(19) United States
(12) Patent Application Publication
(10) Pub. No.: US 2006/0024289 A1
(43) Pub. Date: Feb. 2, 2006

(54) Cleavage of VEGF and VEGF receptor by wild-type and mutant proteases

(76) Inventors: Sandra Waugh Ruggles, South San Francisco, CA (US); Jack Nguyen, South San Francisco, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/104,111

(22) Filed: Apr. 12, 2005

Related U.S. Application Data
(63) Continuation-in-part of application No. 10/677,977, filed on Oct. 2, 2003.
(60) Provisional application No. 60/415,388, filed on Oct. 2, 2002.
(60) Provisional application No. 60/561,720, filed on Apr. 12, 2004.

(51) Int. Cl. C12N 9/42; A61K 38/48; C12N 9/64

(52) U.S. Cl. 424/94.64; 435/226

(57) ABSTRACT

Disclosed herein are methods for creating and using mutein proteases with altered specificity for the target molecules they cleave. The invention further discloses methods of using wild-type and mutein granzyme B to target VEGFR proteins to treat diseases, such as cancer. Cleaving VEGFR at certain substrate sequences with wild-type and mutein granzyme B proteases is a method for treating pathologies associated with angiogenesis.

### RELATED APPLICATIONS

This application claims priority to U.S. Ser. No. 60/561,671 filed Apr. 12, 2004 and claims the benefit of U.S. Ser. No. 10/677,977 filed Oct. 2, 2003 which claims priority to 60/415,388 filed Oct. 2, 2002, which is incorporated herein by reference in its entirety.

### BACKGROUND OF THE INVENTION

The process of angiogenesis is central to the pathology of conditions including malignancy, diabetic retinopathy and macular degeneration. That cancer is angiogenesis-dependent has been recently supported by experimentation in which striking inhibition of tumor growth can be achieved not by direct treatment of the tumor, but rather by selective inhibition of the endothelial growth factor Vascular Endothelial Growth Factor (VEGF). VEGF is an endothelial cell-specific mitogen normally produced during embryogenesis and adult life. VEGF functions as a significant mediator of angiogenesis in a variety of normal and pathological processes, including tumor development. Tumor vascularization is a vital process for the progression of a tumor to a stage from which it can metastasize. Three high affinity cognate VEGF receptors (VEGFRs) have been identified: VEGFR-1/Flt-1, VEGFR-2/KDR, and VEGFR-3/Flt-4. VEGFRs are cell surface receptor tyrosine kinases that function as signaling molecules during vascular development.

An observation common in pre-clinical studies of anti-angiogenic agents targeting VEGF has been potent and broad-spectrum inhibition of very diverse tumor types (solid tissue and hematological), which is consistent with the widespread dependence of cancer on angiogenesis irrespective of tissue of origin. Single i.v. injections of adenoviruses expressing soluble Flk1 and Flt1 transduce the liver, express high plasma levels, and sequester VEGF from its native receptors on endothelial cells. These circulating VEGF receptors produce systemic inhibition of angiogenesis in corneal micropocket assays, and importantly produce strong and broad-spectrum inhibition of tumor angiogenesis and tumor growth in established lung, prostate, colon, brain and pancreas tumors in subcutaneous, orthotopic and transgenic models. See, e.g. Kuo et al. 2001 PNAS 98: 4605-10. Recently, the efficacy of anti-angiogenic therapy has been demonstrated in a randomized phase III trial using the anti-VEGF monoclonal AVASTIN™ (Genentech) to treat patients with metastatic colon cancer, thus providing proof of principle for this treatment strategy in human neoplasia.

### SUMMARY OF THE INVENTION

Nature has engineered the hundreds of proteases in the human genome to exquisite definition so that specificity, inhibition and hydrolysis are perfectly matched to physiological niche. However, there are clear applications of proteases programmed to hydrolyze proteins necessary for cancer growth. This invention pairs structure-based protein engineering techniques with positional scanning synthetic combinatorial library (PSSCL) assays to provide novel serine proteases with specificity that collectively match the VEGF-R2 stalk over an extended region. PSSCL profiling is a proprietary technology that generates a complete substrate specificity profile or “fingerprint” of each engineered protease in a single assay. With this technology, it is now possible to identify therapeutically relevant proteases that have enhanced specificity toward target substrates and little to no activity towards wild-type substrates. In the design process, they produce hundreds of proteases with altered specificity profiles. The technology offers an unprecedented opportunity to study the structural features of specificity. With screening of proteases with PSSCL, the determinants of serine protease selectivity and catalytic efficiency can be identified. They offer not only an opportunity to discover fundamental rules concerning serine protease function, but also additional information for the design of therapeutically relevant molecules.

The present invention provides compositions and methods for using proteases that cleave proteins known to be involved in disease. In particular, wild-type and mutated granzyme B polypeptide (“mutein”) polypeptides are provided that cleave VEGF or VEGF receptor, which is known to be involved in angiogenesis. The resultant modified proteins are provided for use as agents for in vivo therapy of cancers and other angiogenesis-related pathologies, including but not limited to macular degeneration, inflammation and diabetes.

The invention also provides methods for the modification of proteases to alter their substrate sequence specificity, so that the modified proteases specifically cleave a VEGF or VEGF receptor protein. Cleavage of targeted VEGF or VEGFRs is provided for treatment of a broad range of cancers wherein the treatment results in reduction or inhibition of vascularization necessary for continued tumor growth. In one embodiment of the invention, this modified protease is a serine protease. In another embodiment of the invention, this modified protease is a mutein granzyme B or MT-SP1.

One embodiment of the invention involves generating a library of protease sequences to be used to screen for modified proteases that cleave VEGF or VEGFR at a desired substrate sequence. In one aspect of this embodiment, each different member of the protease library is a protease scaffold with at least one mutation made to each member of the library. The remainder of the protease scaffold has the same or a similar sequence to a wild-type protease. The cleavage activity of each member of the library is measured using the desired substrate sequence from the VEGF or VEGFR target protein. As a result, proteases with the highest cleavage activity with regard to the desired substrate sequence are detected.

In another aspect of this embodiment, the number of mutations made to the protease scaffold is 1, 2-5 (e.g. 2, 3, 4 or 5), 5-10 (e.g. 5, 6, 7, 8, 9 or 10), or 10-20 (e.g. 10, 11, 12, 13, 14, 15, 16, 17, 18, 19 or 20). In a preferred embodiment, the mutation(s) confer increased substrate specificity. In a specific embodiment, the mutation(s) are positioned in the scaffold in at least one of the S1, S2, S3 and S4 sites. In certain aspects of this embodiment, the activity of the mutein protease is increased by at least 10-fold, 100-fold, or 1000-fold over the activity of the wild-type protease. In related aspects, the increase is in substrate specificity.

In another embodiment of the invention the members of the library are made up of randomized amino acid sequences, and the cleavage activity of each member of the library by the protease is measured. This type of library is referred to herein as a substrate library. Substrate sequences that are cleaved most efficiently by the protease are detected. In specific aspects of this embodiment, the substrate sequence in a substrate library is 4, 5, 6, 7, 8, 9, 10, 11, 12, 13, 14, 15, 16, 17, 18, 19 or 20 amino acids long.

In another embodiment of the invention, the members of the substrate library are made up of randomized amino acid sequences, and the cleavage selectiveness of each member of the library by the protease is measured. Substrate sequences that are cleaved most selectively by the protease are detected. In specific aspects of this embodiment, the substrate sequence in the substrate library is 4, 5, 6, 7, 8, 9, 10, 11, 12, 13, 14, 15, 16, 17, 18, 19 or 20 amino acids long.

In one embodiment of this example, the specificity is measured by observing how many different substrate sequences the protease cleaves at a given activity. Proteases that cleave fewer substrate sequences at a given activity have greater specificity than those that cleave more substrate sequences.

In one aspect of this embodiment, the substrate sequence is a part of the VEGF or VEGFR target protein. In a specific embodiment, the library peptides include the VEGF or VEGFR residues of the P1, P2, P3 and P4 sites. In another aspect of this embodiment, the efficiency of cleavage by the protease muteins of the invention of the detected substrate sequence is increased by at least 2-fold, at least 5-fold, at least 10-fold, at least 100-fold, or at least 1000-fold over the average activity of the library. In another aspect of this embodiment, the sequence specificity of the MT-SP1 muteins of the invention in cleaving the substrate sequence is increased by at least 10-fold, at least 100-fold, or at least 1000-fold over the cleavage activity of the of wild-type protease. Profiling of wild-type and mutein target specificity may be done by positional scanning substrate combinatorial libraries (PSSCL), as described in PCT publication WO 01/94332, incorporated herein by reference.

In yet another embodiment, the invention provides a method for treating a patient having a VEGF or VEGFR-related pathology, such as cancer, macular degeneration, inflammation and diabetes. The method involves administering to the patient a protease that cleaves a VEGF or VEGFR protein, so that cleaving the VEGF or VEGFR treats the pathology. In a related embodiment, the treatment of cancer by administration of an engineered protease is in combination with treatment with at least one other anti-cancer agent. In one aspect of this embodiment, the protease is an MT-SP1 mutein. In another aspect of this embodiment, the protease is wild-type MT-SP1.

The patient having a pathology, e.g. the patient treated by the methods of this invention, is a mammal, or more particularly, a human.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, controls. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a graphical representation of the results of the PI -Asp-AMC positional scanning synthetic combinatorial library (PSSCL) and individual tetrapeptide kinetics for the grB I99A, N218A, and Y174A variants. The PSSCL activity of each amino acid at the P2, P3 and P4 positions is displayed as a percentage of the activity of the amino acid at that position. The concentration of the granzyme B variant used was adjusted to match that of 50 nM wild-type granzyme B in the library (X for I99A, Y for N218A, and X for Y174A), and the activity was measured over 1 hour. The amino acids represented from left to right of each of the graphs' X-axes are Ala, Arg, Asn, Asp, Gln, Glu, Gly, His, Ile, Leu, Lys, Phe, Pro Ser, Thr, Trp, Tyr, Val and norleucine (Nle). The y-axis reads from bottom to top: 0, 20, 40, 60, 80, 100.

FIG. 2 is a graphic representation of the P1-Asp-AMC positional scanning synthetic combinatorial library (PSSCL) results highlighting the narrow P2 specificity of the I99R variant and the increase in P4-Leu preference for I99F granzyme B. Mutations mimicking homologous protease amino acids at position 99 alter P2 and P4 specificity. The amino acids represented from left to right of each of the graphs' X-axes are Ala, Arg, Asn, Asp, Gln, Glu, Gly, His, Ile, Leu, Lys, Phe, Pro Ser, Thr, Trp, Tyr, Val and norleucine (Nle). The y-axis reads from bottom to top: 0, 20, 40, 60, 80, 100.

FIG. 3 is a graphical representation of the results of the P1-Asp-AMC positional scanning synthetic combinatorial library (PSSCL) for the N218A/R192A and N218A/R192E variants. Arg192 and Asn218 mutations in combination reduce the preference for acidic P3 amino acids. The amino acids represented from left to right of each of the graphs' X-axes are Ala, Arg, Asn, Asp, Gln, Glu, Gly, His, Ile, Leu, Lys, Phe, Pro Ser, Thr, Trp, Tyr, Val and norleucine (Nle). The y-axis reads from bottom to top: 0, 20, 40, 60, 80, 100.

FIG. 4A is a graphical representation of the results of the P1-Asp-AMC positional scanning synthetic combinatorial library (PSSCL) comparing mutein I99A/N218A granzyme B to wild-type to illustrate a broad profile at P4 and P3 positions and a narrow preference for P2-Phe, Tyr and Nor (i.e., norleucine, a nonnatural isostere of methionine). Note that the I99A/N218A granzyme B has dramatically altered extended specificity. The amino acids represented from left to right of each of the graphs' X-axes are Ala, Arg, Asn, Asp, Gln, Glu, Gly, His, Ile, Leu, Lys, Phe, Pro Ser, Thr, Trp, Tyr, Val and norleucine (Nle). The y-axis reads from bottom to top: 0, 20, 40, 60, 80, 100. FIG. 4B is a plot of the free energy (ΔΔG_{T}^{‡}: =−RT ln [(k_{cat}/Kₘ)_{IEPD}/(k_{cat}/Kₘ)_{modified}]) analysis of the N218A/I99A variant compared to wild-type granzyme B shown in FIG. 4A. The N218A/I99A variant exhibits a cooperative effect between the P2 and P4 amino acids and a shift in importance for catalytic activity away from the identity of P4.

FIGS. 5A-5D are graphical representations of the PSSCL results for rat granzyme B. The activity in each subsite well is normalized to 100%. The three dimensional structure of rat granzyme B shown as a ribbon in FIG. 5E and as a surface in FIG. 5F. The N-terminal side of the ecotin inhibitor's substrate-like binding loop is shown in medium gray (lower left residues), and the C-terminal side of the substrate is modeled in light gray (upper right residues).

FIG. 6 is a ribbon diagram of the replaced residues in CB06. The CB06 mutein contains the modified residues I99A/Y174E/N218A, as shown in Table 7.

FIG. 7 is a graphical representation of wild-type grB target specificity at the P2, P3 and P4 positions. An Asp residue at P1 is held constant.

FIG. 8 is a graphical representation of CB01 mutein grB target specificity at the P2, P3 and P4 positions. The CB01 mutein contains the modified residues I99A/N218A, as shown in Table 7. An Asp residue at P1 is held constant.

FIG. 9 is a graphical representation of CB06 mutein grB target specificity at the P2, P3 and P4 positions. An Asp residue at P1 is held constant.

FIG. 10 is a photograph of a protein gel depicting cleavage of VEGF-R2 by wild-type and mutein grB proteases.

FIG. 11 is a graphical representation of grB and grB-Fc activity representing successful secretion of these proteins from mammalian cells transfected with constructs encoding them, as compared to a control assay.

FIG. 12 is a graph of the percent cell proliferation of HUVEC against the log10 of the concentration of rat granzyme B or human granzyme B present to the culture.

FIG. 13A is a photograph of a protein gel depicting granzyme B found in serum of mice injected with adenovirus encoding granzyme B, as different numbers of days post-injection. FIG. 13B is a graph showing the percent inhibition of angiogenesis in a microcorneal in vivo model.

FIG. 14 is a series of graphical representations of a PSSCL profile of wild-type and various mutants of granzyme B showing specificity of various muteins. FIG. 14A depicts the PSSCL profile of of the granzyme B mutein Y151 A (CB143). FIG. 14B depicts the PSSCL profile of of the granzyme B mutein K97Y/I99A (CB125). FIG. 14C depicts the PSSCL profile of of the granzyme B mutein I99A/N218A/R192Y (CB111). FIG. 14D depicts the PSSCL profile of of the granzyme B mutein K97E/I99A/N218A/Y174E (CB121).

FIG. 15 is a photograph of a SDS PAGE gel showing bands of MT-SP1 purified by a one-column purification procedure and then re-folded through successive dialysis steps. MT-SP1 variants were expressed in bacteria and purified from inclusion bodies. Each protease retains high catalytic activity and is >99% pure, and thus are making them appropriate for crystallographic studies.

FIG. 16A-H are a graphical representation of PSSCL profiles of wild-type MT-SP1 and six variants. The MT-SP1 profile (FIG. 2A) shows that its specificity is somewhat broad, such that a variety of amino acids will be accepted in the P4 and P3 positions in addition to Arg or Lys. FIGS. 16B-H are a graphic depiction of PSSCL profiles of MT-SP1 muteins CB18 (FIG. 16B), CB38 (FIG. 16C), CB159 (FIG. 16D), CB83 (FIG. 16E), CB155 (FIG. 16F), CB151 (FIG. 16G), and CB152 (FIG. 16H) showing narrowed specificity profiles. The activity is represented in relative fluorescence units along the y-axis by dividing each amino acid activity by the activity of the best amino acid within each sublibrary.

FIG. 17 is a photograph of a protein gel showing VEGFR2-Fc is efficiently cleaved by wild-type and muteins of MT-SP 1.

FIGS. 18A, 18B and 18C are graphic depictions of the PSSCL substrate specificity profile at P2, P3 and P4, respectively, of human MT-SP1 in a P1-Lys fixed library. The library format for each extended position is listed above the profile. The activity is represented in pM/sec on the y-axis for each amino acid along the x-axis.

FIG. 19 is a graphical representation of trypsin and MT-SP1 protease activity over time in the presence of increasing levels of serum.

FIG. 20 is a graphical representation of the specificity constants for MT-SP1 and the muteins CB18, CB38, CB83, CB151, CB152, CB155 and CB159 on the tetrapeptide synthetic substrates Ac-RQAR-AMC and Ac-RRVR-AMC. The variants are shown along the x-axis while specificity constants are shown along the y-axis.

FIGS. 21A is a graphical representation of the amount of proliferation of endothelial cells treated with increased concentrations of MT-SP1 and the muteins CB18, CB83 and CB152. FIG. 7B is a photograph of a western blot showing the cleavage of VEGFR2 in HUVEC cells in the presence of MT-SP1, CB18 and CB83, respectively. FIG. 7C is a graphical representation of the amount of soluble extracellular VEGFR2 released by HUVECs upon treatment with MT-SP1, CB18 and CB83.

FIG. 22 is a graphical representation of the maximum dose of MT-SP1, CB18 and CB152 that can be tolerated by mice.

FIG. 23 is a graphical representation of the extent of inhibition of neovascularization by a dose of MT-SP1 and CB18.

FIG. 24 is a graphical representation of the inhibition of vascular permeability by MT-SP1, CB18 and CB152 in the mouse Miles assay.

FIG. 25 is a photograph of a protein gel showing the cleavage of VEGF by wild-type MT-SP1 but not the selective variant CB152.

### DETAILED DESCRIPTION OF THE INVENTION

Serine proteases have a highly adaptable protein scaffold. These proteases differ over a broad range in their substrate recognition properties, ranging from highly specific to completely non-specific. Despite these differences in specificity, the catalytic mechanism is well conserved, consisting of a substrate-binding pocket that correctly registers the scissile peptide in the active site. This large family of proteases can be broadly divergent among members in their sequence specificities yet highly conserved in their mechanism of catalysis. This is because substrate specificity is not only determined by local contacts directly between the substrate peptide and the enzyme (first sphere residues), but also by long range factors (second sphere residues). Both first sphere and second sphere substrate binding effects are determined primarily by loops between B-barrel domains. Because these loops are not core elements of the protein, the integrity of the fold is maintained while loop variants with novel substrate specificities can be selected during the course of evolution to fulfill necessary metabolic or regulatory niches at the molecular level.

Laboratory experiments support the theory that the serine proteases are highly adaptable enzymatic scaffolds. For instance, virtually every aspect of subtilisin has been re-engineered, including the enzyme's substrate specificity, thermostability, pH profile, catalytic efficiency, oxidative stability, and catalytic function.

To date, there have been a number of attempts to alter substrate specificity in proteases using structure-guided rational design. One notable example came from the laboratory of Wells and coworkers. Using subtilisin, an enzyme with low specificity for hydrophobic residues at the P1 position, the authors of this reference managed to radically alter its specificity for tribasic residues radically by making 3 point mutations in the substrate binding pocket. The resulting mutant had over a 1000-fold specificity for tribasic substrates versus the original hydrophobic substrate. In total, studies on changing the specificity of proteases suggest it is possible to radically alter substrate specificity radically. This invention discloses specific muteins of proteases having altered target specificity and methods for using them to treat disease.

This invention discloses specific muteins of the serine proteases having altered target specificity for VEGF or VEGFR. Granzyme B is a serine protease (S1-type) necessary for target cell lysis in cell-mediated immune responses. The wild-type protease cleaves after Asp in its consensus recognition site of I/V-E/Q/M-P/T-D (Table 11). Granzyme B is linked to an activation cascade of caspases (aspartate-specific cysteine proteases) responsible for apoptosis execution. Suitable examples of caspases that Granzyme B cleaves include caspase-3, caspase-7, caspase-9 and caspase-10 to give rise to active enzymes mediating apoptosis. We report herein that granzyme B also cleaves the VEGF receptor, a likely target for anti-cancer treatments and other diseases involved in angiogenesis. Membrane-type serine protease 1 (MT-SP1)/matriptase is an epithelial-derived integral membrane enzyme. MT-SP1 is a mosaic protein containing a transmembrane domain, two CUB domains, four LDLR repeats, and a serine protease domain. The protease domain of MT-SP1 has been expressed in bacteria or yeast in milligram quantities and purified. Profiling by positional scanning substrate combinatorial libraries (PSSCL) revealed that it has trypsin-like activity, demonstrating a strong preference for basic residues at the P1 position. The extended P2-P4 specificity of MT-SP1 is shown in Table 16.

MT-SP1 is also a serine protease which has been altered according to the invention for VEGF or VEGFR target specificity

Definition of Terms

Prior to setting forth the invention in detail, certain terms used herein will be defined.

The term “allelic variant” denotes any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term “allelic variant” is also used herein to denote a protein encoded by an allelic variant of a gene.

The term “complements of polynucleotide molecules” denotes polynucleotide molecules having a complementary base sequence and reverse orientation as compared to a reference sequence. For example, the sequence 5′ ATGCACGG 3′ is complementary to 5′ CCGTGCAT 3′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

A “DNA construct” is a single or double stranded, linear or circular DNA molecule that comprises segments of DNA combined and juxtaposed in a manner not found in nature. DNA constructs exist as a result of human manipulation, and include clones and other copies of manipulated molecules.

A “DNA segment” is a portion of a larger DNA molecule having specified attributes. For example, a DNA segment encoding a specified polypeptide is a portion of a longer DNA molecule, such as a plasmid or plasmid fragment, which, when read from the 5′ to the 3′ direction, encodes the sequence of amino acids of the specified polypeptide.

The term “expression vector” denotes a DNA construct that comprises a segment encoding a polypeptide of interest operably linked to additional segments that provide for its transcription in a host cell. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both.

The term “isolated”, when applied to a polynucleotide molecule, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones, as well as synthetic polynucleotides. Isolated DNA molecules of the present invention may include naturally occurring 5′ and 3′ untranslated regions such as promoters and terminators. The identification of associated regions will be evident to one of ordinary skill in the art (see for example, Dynan and Tijan, Nature 316:774-78, 1985). When applied to a protein, the term “isolated” indicates that the protein is found in a condition other than its native environment, such as apart from blood and animal tissue. In a preferred form, the isolated protein is substantially free of other proteins, particularly other proteins of animal origin. It is preferred to provide the protein in a highly purified form, i.e., at least 90% pure, preferably greater than 95% pure, more preferably greater than 99% pure.

The term “operably linked”, when referring to DNA segments, denotes that the segments are arranged so that they function in concert for their intended purposes, e.g. transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

The term “polynucleotide” denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term “nucleotides” is used for both single- and double-stranded molecules where the context permits. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered; thus all nucleotides within a double-stranded polynucleotide molecule may not be paired. Such unpaired ends will, in general, not exceed 20 nt in length.

The term “promoter” denotes a portion of a gene containing DNA sequences that provide for the binding of RNA polymerase and initiation of transcription. Promoter sequences are commonly, but not always, found in the 5′ non-coding regions of genes.

A “protease” is an enzyme that cleaves peptide bonds in peptides, polypeptides and proteins. A “protease precursor” or a “zymogen” is a relatively inactive form of the enzyme that commonly becomes activated upon cleavage by another protease.

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “substrate sequence” denotes a sequence that is cleaved by a protease.

The term “target protein” denotes a protein that is specifically cleaved at its substrate sequence by a protease.

The term “scaffold” refers to a wild-type or existing variant protease to which various mutations are made. Generally, these mutations change the specificity and activity of the scaffold. One example of an existing variant protease is a protease existing in an organism which has been mutated at one or more positions compared to the wild-type protease amino acid sequence of the species to which the organism belongs.

An “isolated” or “purified” polypeptide or protein or biologically-active portion thereof is substantially free of cellular material or other contaminating proteins from the cell or tissue source from which the protein is derived, or substantially free from chemical precursors or other chemicals when chemically synthesized. The language “substantially free of cellular material” includes preparations of proteins in which the protein is separated from cellular components of the cells from which it is isolated or recombinantly-produced. In one embodiment, the language “substantially free of cellular material” includes preparations of protease proteins having less than about 30% (by dry weight) of non-protease proteins (also referred to herein as a “contaminating protein”), more preferably less than about 20% of non-protease proteins, still more preferably less than about 10% of non-protease proteins, and most preferably less than about 5% of non-protease proteins. When the protease protein or biologically-active portion thereof is recombinantly-produced, it is also preferably substantially free of culture medium, i.e., culture medium represents less than about 20%, more preferably less than about 10%, and most preferably less than about 5% of the volume of the protease protein preparation.

The language “substantially free of chemical precursors or other chemicals” includes preparations of protease proteins in which the protein is separated from chemical precursors or other chemicals that are involved in the synthesis of the protein. In one embodiment, the language “substantially free of chemical precursors or other chemicals” includes preparations of protease proteins having less than about 30% (by dry weight) of chemical precursors or non-protease chemicals, more preferably less than about 20% chemical precursors or non-protease chemicals, still more preferably less than about 10% chemical precursors or non-protease chemicals, and most preferably less than about 5% chemical precursors or non-protease chemicals.

The term “serine protease” refers to proteases which contain the serine protease domain. These proteases include members of the serine protease family which are subdivided into structural subclasses (for example S1). Specific examples of serine proteases are provided below.

The term “selectiveness” or “Specificity” is a ratio of efficiency of cleavage of a targeted substrate site versus another substrate site that is not the targeted site.

The term “peptide” refers to a polypeptide of from 2 to 40 amino acids in length.

Protease Scaffolds of the Invention

Protein scaffolds useful in the instant invention include unspecific and specific proteases or fragments of unspecific and specific proteases or are derived from unspecific or specific proteases.

The terms “derived from” or “a derivative thereof” in this respect and in the following variants and embodiments refer to derivatives of proteins that are mutated at one or more amino acid positions and/or have a homology of at least 70%, preferably 90%, more preferably 95% and most preferably 99% to the original protein, and/or that are proteolytically processed, and/or that have an altered glycosylation pattern, and/or that are covalently linked to non-protein substances, and/or that are fused with further protein domains, and/or that have C-terminal and/or N-terminal truncations, and/or that have specific insertions, substitutions and/or deletions. Alternatively, “derived from” may refer to derivatives that are combinations or chimeras of two or more fragments from two or more proteins, each of which optionally comprises any or all of the aforementioned modifications. The tertiary structure of the protein scaffold can be of any type. Preferably, however, the tertiary structure belongs to one of the following structural classes: class S1 (chymotrypsin fold of the serine proteases family), class S8 (subtilisin fold of the serine proteases family), class SC (carboxypeptidase fold of the serine proteases family), class A1 (pepsin A fold of the aspartic proteases), or class C14 (caspase-1 fold of the cysteine proteases).

Examples of proteases that can serve as the protein scaffold of engineered proteases for the use as human therapeutics by cleavage of VEGF or VEGFR are or are derived from granzyme B, MT-SP1, human trypsin, human thrombin, human chymotrypsin, human pepsin, human endothiapepsin, human caspases 1 to 14, and/or human furin.

In a first embodiment, the protein scaffold has a tertiary structure or fold equal or similar to the tertiary structure or fold of the S1 structural subclass of serine proteases, i.e. the chymotrypsin fold, and/or has at least 70% identity on the amino acid level to a protein of the S1 structural subclass of serine proteases. It is preferred that amino acids are altered at one or more positions from the group of positions that correspond structurally or by amino acid sequence homology to the regions 18-25, 38-48, 54-63, 73-86, 122-130, 148-156, 165-171 and 194-204 in human trypsin 1, and more preferably at one or more positions from the group of positions that correspond structurally or by amino acid sequence homology to the regions 20-23, 41-45, 57-60, 76-83, 125-128, 150-153, 167-169, and 197-201. The number of amino acid changes to be combined with this type of protein scaffold is preferably between 1 and 10, and more preferably between 2 and 4.

Preferably, the protein scaffold is equal to or is a derivative or homologue of one or more of the following proteins: chymotrypsin, granzyme, kallikrein, trypsin, mesotrypsin, neutrophil elastase, pancreatic elastase, enteropeptidase, cathepsin, thrombin, ancrod, coagulation factor IXa, coagulation factor vlla, coagulation factor Xa, activated protein C, urokinase, tissue-type plasminogen activator, plasmin, Desmodus-type plasminogen activator. More preferably, the protein scaffold is trypsin or thrombin or is a derivative or homologue from trypsin or thrombin. For the use as a human therapeutic, the trypsin or thrombin scaffold is most preferably of human origin in order to minimize the risk of an immune response or an allergenic reaction.

Preferably, derivatives with improved characteristics derived from human trypsin I or from proteins with similar tertiary structure are used. Preferred examples of such derivatives are derived from human trypsin I and comprise one or more of the following amino acid substitutions E56G; R78W; Y131F; A1 46T; C1 83 R.

In a further embodiment the protein scaffold belongs to the S8 structural subclass of serine proteases and/or has a tertiary structure similar to subtilisin E from Bacillus subtills-and/or has at least 70% identity on the amino acid level to a protein of the S8 structural subclass of serine proteases. Preferably, the scaffold belongs to the subtilisin family or the human pro-protein convertases. It is preferred that amino acids are altered in the protein scaffold at one or more positions from the group of positions that correspond structurally or by amino acid sequence homology to the regions 6-17, 25-29, 47-55, 59-69, 101-111, 117-125, 129-137, 139-154, 158-169, 185-195 and 204-225 in subtilisin E from Bacillus subtilis, and more preferably at one or more positions from the group of positions that correspond structurally or by amino acid sequence homology to the regions 59-69, 101-111, 129-137, 158-169 and 204-225. It is preferred that the protein scaffold is equal to or is a derivative or homologue of one or more of the following proteins: subtilisin Carlsberg; B. subtilis subtilisin E; subtilisin BPN′; B. licheniformis subtilisin; B. lentus subtilisin; Bacillus alcalophilus alkaline protease; proteinase K; kexin; human pro-protein convertase; and human furin. In a preferred variant, Subtilisin BPN′ or one of the proteins SPC 1 to 7 is used as the protein scaffold.

In a further embodiment the protein scaffold belongs to the family of aspartic proteases and/or has a tertiary structure similar to human pepsin. Preferably, the scaffold belongs to the A1 class of proteases and/or has at least 70% identity on the amino acid level to a protein of the A1 class of proteases. It is preferred that amino acids are altered in the protein scaffold at one or more positions from the group of positions that correspond structurally or by amino acid sequence homology to the regions 6-18, 49-55, 74-83, 91-97, 112-120, 126-137, 159-164, 184-194, 242-247, 262-267 and 277-300 in human pepsin, and more preferably at one or more positions from the group of positions that correspond structurally or by amino acid sequence homology to the regions 10-15, 75-80, 114-118, 130-134, 186-191 and 280-296. It is preferred that the protein scaffold is equal to or is a derivative or homologue of one or more of the following proteins: pepsin, chymosin, renin, cathepsin, yapsin. Preferably, pepsin or endothiopepsin or a derivative or homologue thereof is used as the protein scaffold.

In a further embodiment the protein scaffold belongs to the cysteine protease family and/or has a tertiary structure similar to human caspase 7. Preferably the scaffold belongs to the C14 class of cysteine proteases or has at least 70% identity on the amino acid level to a protein of the C14 class of cysteine proteases. It is preferred that amino acids are altered in the protein scaffold at one or more positions from the group of positions that correspond structurally or by amino acid sequence homology to the regions 78-91, 144-160, 186-198, 226-243 and 271-291 in human caspase 7, and more preferably at one or more positions from the group of positions that correspond structurally or by amino acid sequence homology to the regions 80-86, 149-157, 190-194 and 233-238. It is preferred that the protein scaffold is equal to or is a derivative or homologue of one of the caspases 1 to 9.

In a further embodiment the protein scaffold belongs to the S11 class of serine proteases or has at least 70% identity on the amino acid level to a protein of the S11 class of serine proteases and/or has a tertiary structure similar to D-alanyl-D-alanine transpeptidase from Streptomyces species K15. It is preferred that amino acids are altered in the protein scaffold at one or more positions from the group of positions that correspond structurally or by amino acid sequence homology to the regions 67-79, 137-150, 191-206, 212-222 and 241-251 in D-alanyl-D-alanine transpeptidase from Streptomyces species K15, and more preferably at one or more positions from the group of positions that correspond structurally or by amino acid sequence homology to the regions 70-75, 141-147, 195-202 and 216-220 (numbering of amino acids according to SEQ ID NO:15). It is preferred that the D-alanyl-D-alanine transpeptidase from Streptomyces species K15 or a derivative or homologue thereof is used as the scaffold.

In a further embodiment the protein scaffold belongs to the S21 class of serine proteases or has at least 70% identity on the amino acid level to a protein of the S21 class of serine proteases and/or has a tertiary structure similar to assemblin from human cytomegalovirus. It is preferred that amino acids are altered in the protein scaffold at one or more positions from the group of positions that correspond structurally or by amino acid sequence homology to the regions 25-33 64-69, 134-155, 162-169 and 217-244 in assemblin from human cytomegalovirus, and more preferably at one or more positions from the group of positions that correspond structurally or by amino acid sequence homology to the regions 27-31, 164-168 and 222-239. It is preferred that the assemblin from human cytomegalovirus or a derivative or homologue thereof is used as the scaffold.

In a further embodiment the protein scaffold belongs to the S26 class of serine proteases or has at least 70% identity on the amino acid level to a protein of the S26 class of serine proteases and/or has a tertiary structure similar to the signal peptidase from Escherichia coli. It is preferred that amino acids are altered in the protein scaffold at one or more positions from the group of positions that correspond structurally or by amino acid sequence homology to the regions 8-14, 57-68, 125-134, 239-254, 200-211 and 228-239 in signal peptidase from Escherichia coli, and more preferably at one or more positions from the group of positions that correspond structurally or by amino acid sequence homology to the regions 9-13, 60-67, 127-132 and 203-209. It is preferred that the signal peptidase from Escherichia coli or a derivative or homologue thereof is used as the scaffold.

In a further embodiment the protein scaffold belongs to the S33 class of serine proteases or has at least 70% identity on the amino acid level to a protein of the S33 class of serine proteases and/or has a tertiary structure similar to the prolyl aminopeptidase from Serratia marcescens. It is preferred that amino acids are altered in the protein scaffold at one or more positions from the group of positions that correspond structurally or by amino acid sequence homology to the regions 47-54, 152-160, 203-212 and 297-302 in prolyl aminopeptidase from Serratia marcescens, and more preferably at one or more positions from the group of positions that correspond structurally or by amino acid sequence homology to the regions 50-53, 154-158 and 206-210. It is preferred that the prolyl aminopeptidase from Serratia marcescens or a derivative or homologue thereof is used as the scaffold.

In a further embodiment the protein scaffold belongs to the S51 class of serine proteases or has at least 70% identity on the amino acid level to a protein of the S51 class of serine proteases and/or has a tertiary structure similar to aspartyl dipeptidase from Escherichia coli. It is preferred that amino acids are altered in the protein scaffold at one or more positions from the group of positions that correspond structurally or by amino acid sequence homology to the regions 8-16, 38-46, 85-92, 132-140, 159-170 and 205-211 in aspartyl dipeptidase from Escherichia coli, and more preferably at one or more positions from the group of positions that correspond structurally or by amino acid sequence homology to the regions 10-14, 87-90, 134-138 and 160-165. It is preferred that the aspartyl dipeptidase from Escherichia coli or a derivative or homologue thereof is used as the scaffold.

In a further embodiment the protein scaffold belongs to the A2 class of aspartic proteases or has at least 70% identity on the amino acid level to a protein of the A2 class of aspartic proteases and/or has a tertiary structure similar to the protease from human immunodeficiency virus. It is preferred that amino acids are altered in the protein scaffold at one or more positions from the group of positions that correspond structurally or by amino acid sequence homology to the regions 5-12, 17-23, 27-30, 33-38 and 77-83 in protease from human immunodeficiency virus, and more preferably at one or more positions from the group of positions that correspond structurally or by amino acid sequence homology to the regions 7-10, 18-21, 34-37 and 79-82. It is preferred that the protease from human immunodeficiency virus, preferably HIV-1 protease, or a derivative or homologue thereof is used as the scaffold.

In an further embodiment the protein scaffold belongs to the A26 class of aspartic proteases or has at least 70% identity on the amino acid level to a protein of the A26 class of aspartic proteases and/or has a tertiary structure similar to the omptin from Escherichia coli. It is preferred that amino acids are altered in the protein scaffold at one or more positions from the group of positions that correspond structurally or by amino acid sequence homology to the regions 28-40, 86-98, 150-168, 213-219 and 267-278 in omptin from Escherichia coli, and more preferably at one or more positions from the group of positions that correspond structurally or by amino acid sequence homology to the regions 33-85 161-168 and 273-277. It is preferred that the omptin from Escherichia coli or a derivative or homologue thereof is used as the scaffold.

In a further embodiment the protein scaffold belongs to the C1 class of cysteine proteases or has at least 70% identity on the amino acid level to a protein of the C1 class of cysteine proteases and/or has a tertiary structure similar to the papain from Carica papaya. It is preferred that amino acids are altered in the protein scaffold at one or more positions from the group of positions that correspond structurally or by amino acid sequence homology to the regions 17-24, 61-68, 88-95, 135-142, 153-158 and 176-184 in papain from Carica papaya, and more preferably at one or more positions from the group of positions that correspond structurally or by amino acid sequence homology to the regions 63-66, 136-139 and 177-181. It is preferred that the papain from Carica papaya or a derivative or homologue thereof is used as the scaffold.

In a further embodiment the protein scaffold belongs to the C2 class of cysteine proteases or has at least 70% identity on the amino acid level to a protein of the C2 class of cysteine proteases and/or has a tertiary structure similar to human calpain. It is preferred that amino acids are altered in the protein scaffold at one or more positions from the group of positions that correspond structurally or by amino acid sequence homology to the regions 90-103; 160-172, 193-199, 243-260, 286-294 and 316-322 in human calpain-2, and more preferably at one or more positions from the group of positions that correspond structurally or by amino acid sequence homology to the regions 92-101, 245-250 and 287-291. It is preferred that the human calpain-2 or a derivative or homologue thereof is used as the scaffold.

In a further embodiment the protein scaffold belongs to the C4 class of cysteine proteases or has at least 70% identity on the amino acid level to a protein of the C4 class of cysteine proteases and/or has a tertiary structure similar to NIa protease from tobacco etch virus. It is preferred that amino acids are altered in the protein scaffold at one or more positions from the group of positions that correspond structurally or by amino acid sequence homology to the regions 23-31, 112-120, 144-150, 168-176 and 205-218 in NIa protease from tobacco etch virus, and more preferably at one or more positions from the group of positions that correspond structurally or by amino acid sequence homology to the regions 145-149, 169-174 and 212-218. It is preferred that the NIa protease from tobacco etch virus (TEV protease) or a derivative or homologue thereof is used as the scaffold.

In a further embodiment the protein scaffold belongs to the C10 class of cysteine proteases or has at least 70% identity on the amino acid level to a protein of the C10 class of cysteine proteases and/or has a tertiary structure similar to the streptopain from Streptococcus pyogenes. It is preferred that amino acids are altered in the protein scaffold at one or more positions from the group of positions that correspond structurally or by amino acid sequence homology to the regions 81-90, 133-140, 150-164, 191-199, 219-229, 246-256, 306-312 and 330-337 in streptopain from Streptococcus pyogenes, and more preferably at one or more positions from the group of positions that correspond structurally or by amino acid sequence homology to the regions 82-87, 134-138, 250-254 and 331-335. It is preferred that the streptopain from Streptococcus pyogenes or a derivative or hornologue thereof is used as the scaffold.

In a further embodiment the protein scaffold belongs to the C19 class of cysteine proteases or has at least 70% identity on the amino acid level to a protein of the C19 class of cysteine proteases and/or has a tertiary structure similar to human ubiquitin specific protease 7. It is preferred that amino acids are altered in the protein scaffold at one or more positions from the group of positions that correspond structurally or by amino acid sequence homology to the regions 3-15, 63-70, 80-86, 248-256, 272-283 and 292-304 in human ubiquitin specific protease 7, and more preferably at one or more positions from the group of positions that correspond structurally or by amino acid sequence homology to the regions 10-15, 251-255, 277-281 and 298-304. It is preferred that the human ubiquitin specific protease 7 or a derivative or homologue thereof is used as the scaffold.

In a further embodiment the protein scaffold belongs to the C47 class of cysteine proteases or has at least 70% identity on the amino acid level to a protein of the C47 class of cysteine proteases and/or has a tertiary structure similar to the staphopain from Staphylococcus aureus. It is preferred that amino acids are altered in the protein scaffold at one or more positions from the group of positions that correspond structurally or by amino acid sequence homology to the regions 15-23, 57-66, 108-119, 142-149 and 157-164 in staphopain from Staphylococcus aureus, and more preferably at one or more positions from the group of positions that correspond structurally or by amino acid sequence homology to the regions 17-22, 111-117, 143-147 and 159-163. It is preferred that the staphopain from Staphylococcus aureus or a derivative or homologue thereof is used as the scaffold.

In an further embodiment the protein scaffold belongs to the C48 class of cysteine proteases or has at least 70% identity on the amino acid level to a protein of the C48 class of cysteine proteases and/or has a tertiary structure similar to the UIp1 endopeptidase from Saccharomyces cerevisiae. It is preferred that amino acids are altered in the protein scaffold at one or more positions from the group of positions that correspond structurally or by amino acid sequence homology to the regions 40-51, 108-115, 132-141, 173-179 and 597-605 in UIp1 endopeptidase from Saccharomyces cerevisiae, and more preferably at one or more positions from the group of positions that correspond structurally or by amino acid sequence homology to the regions 43-49, 110-113, 133-137 and 175-178. It is preferred that the Ulpl endopepticlase from Saccharomyces cerevisiae or a derivative or homologue thereof is used as the scaffold.

In a further embodiment the protein scaffold belongs to the C56 class of cysteine proteases or has at least 70% identity on the amino acid level to a protein of the C56 class of cysteine proteases and/or has a tertiary structure similar to the Pfpl endopeptidase from Pyrococcus horikoshii. It is preferred that amino acids are altered in the protein scaffold at one or more positions from the group of positions that correspond structurally or by amino acid sequence homology to the regions 8-16, 40-47, 66-73, 118-125 and 147-153 in PfpI endopeptidase from Pyrococcus horikoshii, and more preferably at one or more positions from the group of positions that correspond structurally or by amino acid sequence homology to the regions 9-14, 68-71 120-123 and 148-151. It is preferred that the PfpI endopeptidase from Pyrococcus horikoshii or a derivative or homologue thereof is used as the scaffold.

In a further embodiment the protein scaffold belongs to the M4 class of metallo proteases or has at least 70% identity on the amino acid level to a protein of the M4 class of metallo proteases and/or has a tertiary structure similar to thermolysin from Bacillus thermoproteolyticus. It is preferred that amino acids are altered in the protein scaffold at one or more positions from the group of positions that correspond structurally or by amino acid sequence homology to the regions 106-118, 125-130, 152-160, 197-204; 210-213 and 221-229 in thermolysin from Bacillus thermoproteolyticus, and more preferably at one or more positions from the group of positions that correspond structurally or by amino acid sequence homology to the regions 108-115, 126-129, 199-203 and 223-227. It is preferred that the thermolysin from Bacillus thermoproteolyticus or a derivative or homologue thereof is used as the scaffold.

In a further embodiment the protein scaffold belongs to the M10 class of metallo proteases or has at least 70% identity on the amino acid level to a protein of the M10 class of metallo proteases and/or has a tertiary structure similar to human collagenase. It is preferred that amino acids are altered in the protein scaffold at one or more positions from the group of positions that correspond structurally or by amino acid sequence homology to the regions 2-7, 68-79, 85-90, 107-111 and 135-141 in human collagenase, and more preferably at one or more positions from the group of positions that correspond structurally or by amino acid sequence homology to the regions 3-6, 71-78 and 136-140. It is preferred that human collagenase or a derivative or homologue thereof is used as the scaffold.

Substrate Specificity of Therapeutically Targeted Serine Proteases

Treatment of human disease by therapeutics mostly involves employing small molecules or supplying proteins such as insulin or EPO for specific alterations to cell programs. An important new class of therapeutics being developed is a class of proteases engineered to have new substrate specificity such that they target disease-related molecules. Methods have now been developed to determine the three dimensional structures of proteases that are specificity-programmed to target critical cell surface molecules. Structural data on engineered proteases complexed with target-like peptides provide a framework to understand direct and second shell side chain interactions that determine specificity. The correlation of three dimensional structure and protease activity and specificity are of academic and demonstrated long term clinical interest. The invention moves beyond showing the importance of second shell site alterations in the activity of a protease with altered specificity. See, e.g., Perona, et al. (1995) Biochemistry 34(5): 1489-99.

The invention provides methods of use and methods for designing and testing disease-specific proteases programmed to target VEGF or VEGFR, which is critical in the etiology of cancer and other diseases. These proteases provide an important new approach to the treatment of cancers, e.g., by impeding tumor growth by blocking tumor angiogenesis, as well as other diseases, including but not limited to macular degeneration, inflammation or diabetes, in which angiogenesis plays a causative or contributive role.

The invention also provides methods of use and methods for designing and testing target-specific proteases programmed to target VEGF and VEGFR which are critical for maintaining cancer and other diseases. These proteases provide an important new approach to the treatment of cancers, e.g., by impeding tumor growth by blocking tumor angiogenesis, as well as other diseases, including but not limited to macular degeneration, inflammation or diabetes, in which angiogenesis plays a causative or contributive role.

The invention also provides methods of use and methods for designing and testing angiogenesis-specific proteases programmed to target proteins critical for modulating apoptosis. These proteases provide an important new approach to the treatment of cancers, e.g., by impeding tumor growth by blocking tumor angiogenesis, as well as other diseases, including but not limited to macular degeneration, inflammation or diabetes, in which angiogenesis plays a causative or contributive role.

Methods are provided for specificity determinants in proteases, thereby allowing design of proteases for disabling VEGF or VEGFR. A combination of screening and structure-based mutagenesis is used to design the targeted proteases. Engineering proteases targeted to attack VEGF or VEGFR represents an entirely new sector in the biotechnology industry. Methods are also provided for creating selective proteases as a new therapeutic modality in human disease. Development and proof of concept experiments in animal models of disease provide an understanding of protease substrate selectivity and recognition in this class of enzymes and provide useful information for the dosing and administration of the proteases of the invention for the treatment of human disease.

This disclosure provides protease therapeutic agents, methods for their production and reagents useful therewith. The methods use proteases associated with disease to address growing health concerns such as cardiovascular disease, inflammatory disorders and cancer.

In one embodiment, the invention characterizes the three-dimensional structures of serine proteases with novel extended substrate specificities that are targeted to the vascular endothelial growth factor receptor 2 (VEGF-R2). These proteases were developed using protein engineering and selected using unique and powerful protease profiling technology. Built from wild-type protease scaffolds, they represent a new therapeutic modality in the treatment of cancer.

Signaling by vascular endothelial growth factor (VEGF) and its receptors is implicated in pathological angiogenesis and the rapid development of tumor vasculature in cancer. Drugs that block this signaling pathway prevent the growth and maintenance of tumor blood supply, which leads to the systematic death of the tumor. The recent success of the anti-VEGF antibody AVASTIN™ in patients with metastatic colon cancer has validated VEGF as a target for anti-angiogenic therapy of cancer. Despite these encouraging results, tumor progression has still occurred in anti-VEGF treatment.

The AVASTIN™ antibody binds VEGF and prevents it from binding to its receptor. Knock-down experiments show that blocking VEGF function blocks angiogenesis. Thus the inhibition of angiogenic signaling through VEGFR-2 represents an underdeveloped therapeutic area ideal for the development of engineered proteases with novel targeting.

Treatment with a protease that specifically cleaves and inactivates the signaling of the VEGF/VEGFR-2 complex will attenuate the angiogenic signal and create a pool of soluble receptor that lowers free VEGF levels. Variant proteases have an in vitro specificity that recognizes a critical region of the VEGF receptor, which is, in one embodiment, the Flk-1/KDR stalk, over a six amino acid region. Due to their catalytic nature and smaller size, engineered proteases provide a new therapeutic treatment with advantages over competing targeted binding proteins. The advantages are: better tumor penetration, better target saturation, higher effectiveness, and potentially lower dosing. Notably, because they bind, hydrolyze, and release, a single protease could cleave and inactivate hundreds to thousands of substrate VEGF receptors, offering substantial therapeutic amplification. Further, some wild-type proteases also cleave VEGFR, and are also used according to the invention. For example, wild-type MT-SP1 cleaves VEGFR.

Structural data on engineered proteases complexed with target-like peptides provide a framework to understand direct (first) and less direct (second) shell side chain interactions that proscribe specificity. The three dimensional structures of proteases determine its specificity. Residues in the protease scaffold that are important in establishing first and second shell interactions with its substrate are mutated to program the protease to attack various cell surface molecules of interest. The correlation of three dimensional structure and protease activity and specificity provide the following:
1. design and characterization of engineered serine proteases with altered substrate specificity;
2. production and characterization of mutated variants of the macromolecular protease inhibitor ecotin that are mated in specificity to the engineered proteases with substrates and chloromethyl ketone inhibitors;
3. crystallization and determination of the three-dimensional structures for the protease- ecotin complexes; and
4. analysis of the protease-ecotin structures, with subsequent creation of a library of protease and substrate interactions important for selectivity and catalysis.

Structures of proteases without substrate analogs are far less helpful than those of proteases with substrate analogs. Substrate analogs are available that bind to many proteases. One powerful agent is ecotin, a serine protease inhibitor found in the periplasm of Escherichia coli. It is a 142-amino acid protein, a novel inhibitor for virtually all serine proteases, regardless of sequence specificity [McGrath, J Biol Chem. 1991 Apr. 5;266(10):6620-5.]. Ecotin's scissile Met84-Met85 bond lies within a disulfide-bonded protein segment similar to other classes of inhibitors, allowing ecotin to remain bound to the protease even if it is cut.

The pan-specificity of inhibition by ecotin derives from formation of a heterotetrameric complex with target proteases involving three types of interface; the dimerization interface, a primary substrate-like interaction, and a smaller secondary interaction between the partner ecotin subunit and the proteases. Ecotin accommodates different shapes of proteases by a balance of the primary and the secondary site. The effect of the secondary binding site on affinity was found to vary inversely with the strength of the interaction at the primary site. This compensatory effect yields a nonadditivity of up to 5 kcal/mol. This remarkable mechanism of adaptability allows femtomolar affinities for two proteases with unrelated specificities [Eggers, J Mol Biol. 2001 May 18;308(5):975-91.].

Ecotin complexes were used to distinguish the related specificities of Factor Xa and thrombin [Wang et al., Biochemistry 2001 Aug. 28;40(34): 10038-46]. Ecotin fails to inhibit thrombin despite its broad specificity. A point mutation (M84R) in ecotin results in a 1.5 nM affinity for thrombin, which is 10e4 times stronger than that of wild-type ecotin. The crystal structure of the mutant complex showed surface loops surrounding the active site of thrombin with structural changes to permit inhibitor binding [Wang et al., Biochemistry 2001 Aug. 28;40(34): 10038-46]. The insertion loops at residues 60 and 148 in thrombin move when the complex forms. Important for a structural read-out of specificity, the active site of thrombin is filled with eight consecutive amino acids of ecotin and demonstrates thrombin's preference for specific features: namely, negatively charged Pro-Val-X-Pro-Arg-hydrophobic-positively charged (SEQ ID NO:19). The preference for a Val at P4 is clearly defined.

The reasons for ecotin's adjustment to inhibit proteases is well understood [Gillmor, J Mol Biol. 2000 Jun. 16;299(4):993-1003]. In contrast to thrombin, the serine protease factor Xa (FXa) is inhibited by native ecotin. The structure of the tetrameric complex of ecotin variant M84F with FXa shows that a substrate-directed induced fit of the binding interactions at the S2 and S4 pockets modulates the discrimination of protease [Wang, Biochemistry. 2003 Jul. 8;42(26):7959-66.]. The Tyr at position 99 of FXa changes its conformation with respect to incoming ligand, thereby changing the size of the S2 and S4 pockets. The role of residue 192 in substrate and inhibitor recognition was shown to be general in mediating the interactions these coagulation proteases and their inhibitors.

Ecotin was used to identify the 11 subsites of fiddler crab collagenase and has been accepted as a beneficial agent not only to provide readout of specificities but also to help crystallize difficult proteases [Perona, J. J., Tsu, C. A., Craik, C. S., and Fletterick, R. J. (1997) Biochemistry 36(18), 5381-92; Tsu, Biochemistry. 1997 May 6;36(18):5393-401]. Chloromethyl ketones are the traditional approach to imaging substrate analogs in proteases and can be used like ecotin to reveal the subsite interaction between a substrate analog and a protease up to the scissile bond. Over sixty structures of these complexes has been determined and, like ecotin, help define the protease specificity mechanism.

VEGF-R2 and Angiogenic Pathology

Vascular endothelial growth factor (VEGF) is a cytokine that binds and signals through a specific cell surface receptor (VEGFR) to regulate angiogenesis, the process in which new blood vessels are generated from existing vasculature. Pathological angiogenesis describes the increased vascularization associated with disease and includes events such as the growth of solid tumors [McMahon, Oncologist. 2000;5 Suppl 1:3-10], macular degeneration and diabetes. In cancer, solid tumors require an ever-increasing blood supply for growth and metastasis. Hypoxia or oncogenic mutation increases the levels of VEGF and VEGF-R mRNA in the tumor and surrounding stromal cells leading to the extension of existing vessels and formation of a new vascular network. In wet macular degeneration, abnormal blood vessel growth forms beneath the macula. These vessels leak blood and fluid into the macula damaging photoreceptor cells. In diabetes, a lack of blood to the eyes can also lead to blindness. VEGF stimulation of capillary growth around the eye leads to disordered vessels which do not function properly.

Three tyrosine kinase family receptors of VEGF have been identified (VEGF-R-1/Flt-1, VEGF-R-2/Flk-1/KDR, VEGF-R-3/Flt-4.) KDR (the mouse homolog is Flk-1) is a high affinity receptor of VEGF with a Kd of 400-800 pM [Waltenberger, J Biol Chem. 1994 Oct. 28; 269(43):26988-95] expressed exclusively on endothelial cells. VEGF and KDR association has been identified as a key endothelial cell-specific signaling pathway required for pathological angiogenesis [Kim, Nature. 1993 Apr. 29; 362 (6423):841-4; Millauer, Nature. 1994 Feb. 10; 367 (6463):576-9; Yoshiji, Hepatology. 1999 November; 30(5): 1179-86]. Dimerization of the receptor upon ligand binding causes autophosphorylation of the cytoplasmic domains, and recruitment of binding partners that propagate signaling throughout the cytoplasm and into the nucleus to change the cell growth programs. Treatment of tumors with a soluble VEGF-R2 inhibits tumor growth [Lin, Cell Growth Differ. 1998 January; 9(1):49-58], and chemical inhibition of phosphorylation causes tumors cells to become apoptotic [Shaheen, Cancer Res. 1999 Nov. 1; 59(21):5412-6].

Therapies targeting the VEGF receptors and Flk-1/KDR specifically have inhibited pathological angiogenesis and shown reduction of tumor size in multiple mouse models of human and mouse solid tumors [Prewett, Cancer Res. 1999 Oct. 15; 59(20):5209-18; Fong, Neoplasia. 1999 April; 1(1):31-41. Erratum in: Neoplasia 1999 June;1(2):183] alone and in combination with cytotoxic therapies [Klement, J Clin Invest. 2000 April; 105(8):R15-24]. Studies with small molecule inhibitors and antibodies validate the VEGF receptor family as a potent anti-angiogenesis target but leave room for a more effective therapeutics are still needed.

VEGFR is composed of an extracellular region of seven immunoglobin (Ig)-like domains, a transmembrane region, and two cytoplasmic tyrosine kinase domains. The first three Ig-like domains have been shown to regulate ligand binding, while domains 4 through 7 have a role in inhibiting correct dimerization and signaling in the absence of ligand. As a target for selective proteolysis by engineered proteases, it has the following promising target characteristics:
a labile region of amino acids accessible to proteolysis;
high sequence identity between the human, rat and mouse species;
down regulation of signaling upon cleavage; and
proteolytic generation of soluble receptors able to non-productively bind ligand.

Several regions of VEGF-R2 are available for specific proteolysis including the stalk region before the transmembrane region and unstructured loop between Ig-like domains.

The present invention provides methods for generating and screening proteases to cleave target proteins at a given substrate sequence as well as particular muteins and methods for using them to treat disease. Proteases are protein-degrading enzymes that recognize an amino acid or an amino acid substrate sequence within a target protein. Upon recognition of the substrate sequence, proteases catalyze the hydrolysis or cleavage of a peptide bond within a target protein. Such hydrolysis of the target protein can inactivate it, depending on the location of peptide bond within the context of the full-length sequence of the target sequence. The specificity of proteases can be altered through protein engineering. If a protease is engineered to recognize a substrate sequence within a target protein or proteins (i) that would alter the function i.e. by inactivation of the target protein(s) upon catalysis of peptide bond hydrolysis, and (ii) the target protein(s) are recognized or unrecognized is a point of molecular intervention for a particular disease or diseases, then the engineered protease has a therapeutic effect via a proteolysis-mediated inactivation event. In particular, serine-like proteases (e.g. granzyme B and MT-SP1) can be engineered to cleave specific target receptors between their transmembrane and cytokine or growth factor binding domains. The stalk regions that function to tether protein receptors to the surface of a cell or loop regions are thereby disconnected from the globular domains in a polypeptide chain.

The protease cleaves a VEGF or VEGFR which are responsible for modulation of angiogenesis. Where the cell surface molecule is a VEGFR signaling in tumor angiogenesis, cleavage prevents the spread of cancer. For example, cleavage of a cell surface domain from a VEGFR molecule can inactivate its ability to transmit extracellular signals, especially cell proliferation signals. Without angiogenesis to feed the tumor, cancer cells often cannot proliferate. In one embodiment, a granzyme B protease of the invention is therefore used to treat cancer. Also, cleavage of VEGFR can be used to modulate angiogenesis in other pathologies, such as macular degeneration, inflammation and diabetes. In one embodiment, cleaving a target VEGF or VEGFR protein involved in cell cycle progression inactivates the ability of the protein to allow the cell cycle to go forward. Without the progression of the cell cycle, cancer cells can not proliferate. Therefore, the proteases of the invention which cleave VEGF or VEGFR are used to treat cancer and other cell cycle dependent pathologies.

The protease also cleaves soluble proteins that are responsible for tumorigenicity. Cleaving VEGF prevents signaling through the VEGF receptor and decreases angiogenesis, thus decreasing disease in which angiogenesis plays a role, such as cancer, macular degeneration, inflammation and diabetes. Further, VEGF signaling is responsible for the modulation of the cell cycle in certain cell types. Therefore, the MT-SP1 proteases of the invention which cleave VEGF are useful in the treatment of cancer and other cell cycle dependent pathologies.

In some embodiments, the engineered granzyme B protease is designed to cleave one or more of the target proteins in Table 1, thereby inactivating the activity of the protein. The granzyme B protease can be used to treat a pathology associated with that protein, by inactivating it.

TABLE 1
Protease Targets

| Target | Indication | Molecule class |
|---|---|---|
| VEGF | Cancer | Cytokine |
| VEGFR-1/Flt-1 | Cancer | Receptor |
| VEGFR-2/KDR | Cancer | Receptor |
| VEGFR-3/Flt-4 | Cancer | Receptor |

Granzyme B Multiple sequence alignment of the granzyme B related serine proteases was performed and shown in Table 2. Serine proteases related to granzyme B by distinct active site architecture (no disulfide at amino acids 191 and 220, a truncated 220s loop compared to trypsin, and a cis-Pro at 224) were found. Sequence accession codes from GenBank are reported and the Protein Data Bank identifiers are shown in italics where structures are available. The amino acids with side chains within close contact (<4 Å) of the substrate are labeled and shown with highlighting.

The wild-type granzyme B protease of the invention is provided as SEQ ID NO:1. Granzyme B belongs to the granzyme subfamily of serine proteases. A ClustalW 5 alignment of mature wild-type human granzyme B (GRAB HUMAN) and other granzyme subfamily members beginning with the canonical N-terminus is provided in Table 2.

[image]

[image]

The granzyme B polypeptide is encoded by the GZMB gene, which resides at chromosome locus 14q11.2. The human and rat granzyme B precursor polypeptide of SEQ ID NO:20 and 21 are provided in Table 3. The signal sequence that is cleaved prior to activation is underlined. The GenBank accession number for the human granzyme B protein is M17016 (or 338295) and for rat granzyme B is M34097 (NM_{—}138517). The wild-type mature granzyme B protease of the invention includes residues 21-247 of SEQ ID NOS:20 and 21.

TABLE 3

Wild-type human (SEQ ID NO:20) and rat (SEQ ID NO:21) granzyme B
precursor proteins

(SEQ ID NO:20)

MQPILLLLAF LLLPRADAGE IIGGHEAKPH SRPYMAYLMI WDQKSLKRCG GFLIQDDFVL
TAAHCWGSSI NVTLGAHNIK EQEPTQQFIP VKRPIPHPAY NPKNFSNDIM LLQLERKAKR
TRAVQPLRLP SNKAQVKPGQ TCSVAGWGQT APLGKHSHTL QEVKMTVQED RKCESDLRHY
YDSTIELCVG DPEIKKTSFK GDSGGPLVCN KVAQGIVSYG RNNGMPPRAC TKVSSFVHWI
KKTMKRY

(SEQ ID NO:21)

MKLLLLLLSF SLAPKTEAGE IIGGHEAKPH SRPYMAYLQI MDEYSGSKKC GGFLIREDFV
LTAAHCSGSK INVTLGAHNI KEQEKMQQII PVVKIIPHPA YNSKTISNDI MLLKLKSKAK
RSSAVKPLNL PRRNVKVKPG DVCYVAGWGK LGPMGKYSDT LQEVELTVQE DQKCESYLKN
YFDKANEICA GDPKIKRASF RGDSGGPLVC KKVAAGIVSY GQNDGSTPRA FTKVSTFLSW
IKKTMKKS

A ClustalW alignment is provided in Table 4, comparing the protease domain of the wild-type human granzyme B polypeptide of SEQ ID NO:1, to the rat granzyme B protease domain of SEQ ID NO:21. Important granzyme B protease domain residues are shown in bold. The bold amino acids are at the specificity determinants, as discussed in more detail below.

TABLE 4
ClustalW of granzyme B Protease Domain

```
Pileup - granzyme B protease domain (grB_dom)
      MSF: 228 Type: P Check: 2604 ..
  Name: Human_grB_dom Len: 228 Check: 8002 Weight: 0
  Name: Rat_grB_dom Len: 228 Check: 4602 Weight: 0
//
```

| | | |
|---|---|---|
| | 1 50 | (SEQ ID NO:1) |
| Human_grB_dom | IIGGHEAKPH SRPYMAYLMI WDQKS.LKRC GGFLIQDDFV LTAAHCWGSS | |
| Rat_grB_dom | IIGGHEAKPH SRPYMAYLQI MDEYSGSKKC GGFLIREDFV LTAAHCSGSK | |
| | 51 100 | (SEQ ID NO:21) |
| Human_grB_dom | INVTLGAHNI KEQEPTQQFI PVKRPIPHPA YNPKNFSNDI MLLQLERKAK | |
| Rat_grB_dom | INVTLGAHNI KEQEKMQQII PVVKIIPHPA YNSKTISNDI MLLKLKSKAK | |
| | 101 150 | |
| Human_grB_dom | RTRAVQPLRL PSNKAQVKPG QTCSVAGWGQ TAPLGKHSHT LQEVKMTVQE | |
| Rat_grB_dom | RSSAVKPLNL PRRNVKVKPG DVCYVAGWGK LGPMGKYSDT LQEVELTVQE | |
| | 151 200 | |
| Human_grB_dom | DRKCESDLRH YYDSTIELCV GDPEIKKTSF KGDSGGPLVC NKVAQGIVSY | |
| Rat_grB_dom | DQKCESYLKN YFDKANEICA GDPKIKRASF RGDSGGPLVC KKVAAGIVSY | |
| | 201 228 | |
| Human_grB_dom | GRNNGMPPRA CTKVSSFVHW IKKTMKRY | |
| Rat_grB_dom | GQNNGSTPRA FTKVSTFLSW IKKTMKKS | |

MT-SP1

TABLE 5
Protease Scaffolds

| Code | Name | Gene | Link | Locus |
|---|---|---|---|---|
| S01.087 | membrane-type serine protease | MT-SP1 | 84000 | 11q23 |

The wild-type MT-SP1 polypeptide of SEQ ID NO:22 is provided in Table 6, and is designated as TADG-15.

TABLE 6
Wild-type MT-SP1 polypeptide (SEQ ID NO:22)

| | | |
|---|---|---|
| | 1 50 | |
| TADG-15 | MGSDRARKGG GGPKDFGAGL KYNSRHEKVN GLEEGVEFLP VNNVKKVEKH | (SEQ ID NO:22) |
| | 51 100 | |
| TADG-15 | GPGRWVVLAA VLIGLLLVLL GIGFLVWHLQ YRDVRVQRVF NGYMRITNBN | |
| | 101 150 | |
| TADG-15 | FVDAYENSNS TEFVSLASKV KDALKLLYSG VPFLGPYHKE SAVTAFSEGS | |
| | 151 200 | |
| TADG-15 | VIAYYWSEFS IPQHLVEEAE RVMAEERVVN LPPRARSLKS FVVTSVVAFP | |
| | 201 250 | |
| TADG-15 | TDSKTVQRTQ DNSCSFGLHA RGVELMRFTT PGFPDSPYPA HARCQWALRG | |
| | 251 300 | |
| TADG-15 | DADSVLSLTF RSFDLASCDE RGSDLVTVYN TLSPMEPHAL VQLCGTYPPS | |
| | 301 350 | |
| TADG-15 | YNLTFHSSQN VLLITLITNT ERRHPGFEAT FFQLPRNSSC GGRLRKAQGT | |
| | 351 400 | |
| TADG-15 | FNSPYYPGHY PPNIDCTWNI EVPNNQHVKV SFKFFYLLEP GVPAGTCPKD | |
| | 401 450 | |
| TADG-15 | YVEINGEKYC GERSQFVVTS NSNKITVRFH SDQSYTDTGF LAEYLSYDSS | |
| | 451 500 | |
| TADG-15 | DPCPGQFTCR TGRCIRKELR CDGWADCTDH SDELNCSCDA GHQFTCKNKF | |
| | 501 550 | |
| TADG-15 | CKPLFWVCDS VNDCGDNSDE QGCSCPAQTF RCSNGKCLSK SQQCNGKDDC | |
| | 551 600 | |
| TADG-15 | GDGSDEASCP KVNVVTCTKH TYRCLNGLCL SKGNPECDGK EDCSDGSDEK | |
| | 601 650 | |
| TADG-15 | DCDCGLRSPT RQARVVGGTD ADEGEWPWQV SLHALGQGHI CGASLISPNW | |
| | 651 700 | |
| TADG-15 | LVSAAHCYID DRGFRYSDPT QWTAFLGLHD QSQRSAPGVQ ERRLKRIISH | |
| | 701 750 | |
| TADG-15 | PFFNDFTFDY DIALLELEKP AEYSSMVRPI CLPDASHVFP AGKAIWVTGW | |
| | 751 800 | |
| TADG-15 | GHTQYGGTGA LILQKGEIRV INQrTCENLL PQQITPRMMC VGFLSGGVDS | |
| | 801 850 | |
| TADG-15 | CQGDSGGPLS SVEADGRIFQ AGVVSWGDGC AQRNKPGVYT RLPLFRDWIK | |
| TADG-15 | ENTGV | |

A ClustalW alignment is provided in Table 7, comparing the wild-type MT-SP1 polypeptide of SEQ ID NO:22, designated as TADG-15, to the MT-SP1 protease domain of SEQ ID NO:23. MT-SP1 protease domain residues targeted for mutagenesis are shown in bold. The MT-SP1 protease domain is composed of a pro-region and a catalytic domain. The catalytically active portion of the sequence begins after the autoactivation site: RQAR followed by the sequence VVGG (underlined).

TABLE 7
ClustalW of MT-SP1 Protease Domain

```
Pileup
      MSF: 855 Type: P Check: 4738 ..
  Name: MT-SP1_protease_domain Len: 855 Check: 8683 Weight: 0
  Name: TADG-15 Len: 855 Check: 6055 Weight: 0
//
```

| | | |
|---|---|---|
| | 1 50 | |
| MTSP_protease_domain | .......... .......... .......... .......... .......... | (SEQ ID NO:23) |
| TADG-15 | MGSDRARKGG GGPKDFGAGL KYNSRHEKVN GLEEGVEFLP VNNVKKVEKH | (SEQ ID NO:22) |
| | 51 100 | |
| MTSP_protease_domain | .......... .......... .......... .......... .......... | |
| TADG-15 | GPGRWVVLAA VLIGLLLVLL GIGFLVWHLQ YRDVRVQKVF NGYMRITNEN | |
| | 101 150 | |
| MTSP_protease_domain | .......... .......... .......... .......... .......... | |
| TADG-15 | FVDAYENSNS TEFVSLASKV KDALKLLYSG VPFLGPYHKE SAVTAFSEGS | |
| | 151 200 | |
| MTSP_protease_domain | .......... .......... .......... .......... .......... | |
| TADG-15 | VIAYYWSEFS IPQHLVEEAE RVMAEERVVM LPPRARSLKS FVVTSVVAFP | |
| | 201 250 | |
| MTSP_protease_domain | .......... .......... .......... .......... .......... | |
| TADG-15 | TDSKTVQRTQ DNSCSFGLHA RGVELMRFFT PGFPDSPYPA HARCQWALRG | |
| | 251 300 | |
| MTSP_protease_domain | .......... .......... .......... .......... .......... | |
| TADG-15 | DADSVLSLTF RSFDLASCDE RGSDLVTVYN TLSPMEPHAL VQLCGTYPPS | |
| | 301 350 | |
| MTSP_protease_domain | .......... .......... .......... .......... .......... | |
| TADG-15 | YNLTFHSSQN VLLITLITNT ERRHPGFEAT FFQLPRNSSC GGRLRKAQGT | |
| | 351 400 | |
| MTSP_protease_domain | .......... .......... .......... .......... .......... | |
| TADG-15 | FNSPYYPGHY PPNIDCTWNI EVPNNQWJKV SFKFFYLLEP GVPAGTCPKD | |
| | 401 450 | |
| MTSP_protease_domain | .......... .......... .......... .......... .......... | |
| TADG-15 | YVEINGEKYC GERSQFVVTS NSNKITVRFH SDQSYTDTGF LAEYLSYDSS | |
| | 451 500 | |
| MTSP_protease_domain | .......... .......... .......... .......... .......... | |
| TADG-15 | DPCPGQFTCR TGRCIRKELR CDGWADCTDH SDELNCSCDA GHQFTCKNKF | |
| | 501 550 | |
| MTSP_protease_domain | .......... .......... .......... .......... .......... | |
| TADG-15 | CKPLFWVCDS VNDCGDNSDE QGCSCPAQTF RCSNGKCLSK SQQCNGKDDC | |
| | 551 600 | |
| MTSP_protease_domain | .......... .......... .......... .......... .......DEK | |
| TADG-15 | GDGSDEASCP KVNVVTCTKH TYRCLNGLCL SKGNPECDGK EDCSDGSDEK | |
| | 601 650 | |
| MTSP_protease_domain | DCDCGLRSFT RQARVVGGTD ADEGEWPWQV SLHALGQGHI CGASLISPNW | |
| TADG-15 | DCDCGLRSFT RQARVVGGTD ADEGEWPWQV SLHALGQGHI CGASLISPNW | |
| | 651 700 | |
| MTSP_protease_domain | LVSAAHCYID DRGFRYSDPT QWTAFLGLHD QSQRSAPGVQ ERRLKRIISH | |
| TADG-15 | LVSAAHCYID DRGFRYSDPT QWTAFLGLHD QSQRSAPGVQ ERRLKRIISH | |
| | 701 750 | |
| MTSP_protease_domain | PFFNDPTFDY DIALLELEKP ABYSSMVRPI CLPDASHVFP AGKAIWVTGW | |
| TADG-15 | PFFNDFTFDY DIALLELEEP AEYSSMVRPI CLPDASHVFP AGKAIWVTGW | |
| | 751 800 | |
| MTSP_protease_domain | GHTQYGGTGA LILQKGEIRV INQTTCENLL PQQITPRMMC VGFLSGGVDS | |
| TADG-15 | GHTQYGGTGA LILQKGEIRV INQTTCENLL PQQITPRNMC VGFLSGGVDS | |
| | 801 850 | |
| MTSP_protease_domain | CQGDSGGPLS SVEADGRIFQ AGVVSWGDGC AQRNKPGVYT RLPLFRDWIK | |
| TADG-15 | CQGDSGGPLS SVEADGRIFQ AGVVSWGDGC AQRNKPGVYT RLPLFRDWIK | |
| | 851 | |
| MTSP_protease_domain | ENTGV | |
| TADG-15 | ENTGV | |

A ClustalW alignment is provided in Table 8, comparing the wild-type MT-SP1 protease domain of SEQ ID NO:23 with human chymotrypsin (SEQ ID NO:24). MT-SP1 protease domain residues targeted for mutagenesis are numbered according to chymotrypsin.

TABLE 8
Clustal W alignment of human chymotrypsin and MT-SP1 protease domain

| | | |
|---|---|---|
| | 16 30 31 45 46 60 61 66 | |
| Chymotrypsin B | IVNGEDAVPGSWPWQ VSLQDKTGFHFCGGS LISEDWVVTAAHCGV ---------RTSDVV | (SEQ ID NO:24) |
| MTSP_protease_domain | VVGGTDADEGEWPWQ VSLHALGQGHICGAS LISPNWLVSAAHCYI DDRGFRYSDPTQWTA | (SEQ ID NO:23) |
| | 67 80 81 95 96 110 111 125 | |
| Chymotrypsin B | VAGEFDQGS-DEENI QVLKIAKVFKNPKFS ILTVNNDITLLKLAT PARFSQTVSAVCLPS | |
| MTSP_protease_domain | FLGLHDQSQRSAPGV QERRLKRIISHPFFN DFTFDYDIALLELEK PAEYSSMVRPICLPD | |
| | 126 140 141 155 156 170 171 184 | |
| Chymotrypsin B | ADDDFPAGTLCATDG WGKTKYNANKTPDKL QQAALPLLSNAECKK SWGRRITDVMICAG- | |
| MTSP_protease_domain | ASNVFPAGKAIWVTG WGHTQYGG-TGALIL QKGEIRVINQTTCEN LLPQQITPRNMCVGF | |
| | 185 198 199 212 213 226 227 240 | |
| Chymotrypsin B | -ASGVSSCMGDSGGP L-VCQKDGAWTLVGI VSWGSDTCSTSS-PG VYARVTKLIPWVQKI | |
| MTSP_protease_domain | LSGGVDSCQGDSGGP LSSVEADGRIFQAGV VSWGDG-CAQRNKPG VYTRLPLFRDWIKEN | |
| | 241 | |
| Chymotrypsin B | LAAN | |
| MTSP_protease_domain | TGV- | |

A DNA sequence is provided in Table 9 which encodes the catalytic domain (SEQ ID No:19) of wild-type MT-SP1 protease domain as contained within the pQE cloning vector.

TABLE 9
The DNA sequence of the catalytic domain of wild-type MT-SP1.

| | |
|---|---|
| gtt gtt ggg ggc acg gat gcg gat gag ggc gag tgg ccc tgg cag gta agc ctg cat gct | (SEQ ID NO:19) |
| ctg ggc cag ggc cac atc tgc ggt gct tcc ctc atc tct ccc aac tgg ctg gtc tct gcc | |
| gca cac tgc tac atc gat gac aga gga ttc agg tac tca gac ccc acg cag tgg acg gcc | |
| ttc ctg ggc ttg cac gac cag agc cag cgc agc gcc cct ggg gtg cag gag cgc agg ctc | |
| aag cgc atc atc tcc cac ccc ttc ttc aat gac ttc acc ttc gac tat gac atc gcg ctg | |
| ctg gag ctg gag aaa ccg gca gag tac agc tcc atg gtg cgg ccc atc tgc ctg ccg gac | |
| gcc tcc cat gtc ttc cct gcc ggc aag gcc atc tgg gtc acg ggc tgg gga cac acc cag | |
| tat gga ggc act ggc gcg ctg atc ctg caa aag ggt gag atc cgc gtc atc aac cag acc | |
| acc tgc gag aac ctc ctg ccg cag cag atc acg ccg cgc atg atg tgc gtg ggc ttc ctc | |
| agc ggc ggc gtg gac tcc tgc cag ggt gat tcc ggg gga ccc ctg tcc agc gtg gag gcg | |
| gat ggg cgg atc ttc cag gcc ggt gtg gtg agc tgg gga gac ggc tgc gct cag agg aac | |
| aag cca ggc gtg tac aca agg ctc cct ctg ttt cgg gac tgg atc aaa gag aac act ggg | |
| gta tag | |

Engineering Proteases.

Virtually every aspect of a protease can be re-engineered, including the enzyme substrate sequence specificity, thermostability, pH profile, catalytic efficiency, oxidative stability, and catalytic function.

Wild-type protease is used in accordance with the methods of the invention as a scaffold for incorporating various mutations that change its substrate specificity. Among the determinants of substrate sequence specificity in serine proteases come from the S1-S4 positions in the active site, where the protease is in contact with the P1-P4 residues of the peptide substrate sequence. In some cases, there is little (if any) interaction between the S1-S4 pockets of the active site, such that each pocket appears to recognize and bind the corresponding residue on the peptide substrate sequence independent of the other pockets. Thus the specificity determinants may be generally changed in one pocket without affecting the specificity of the other pockets.

For example, a protease with low specificity for a residue at a particular binding site or for a particular sequence is altered in its specificity by making point mutations in the substrate sequence binding pocket. In some cases, the resulting protease mutein has a greater than 2-fold increase in specificity at a site or for a particular sequence than does wild-type. In another embodiment, the resulting protease mutein has a greater than 5-fold increase in specificity at a site or for a particular sequence than does wild-type. In another embodiment, the resulting protease mutein has a greater than 10-fold increase in specificity at a site or for a particular sequence than does wild-type. In another embodiment, the resulting protease mutein has a greater than 100-fold increase in specificity at a site or for a particular sequence than does wild-type. In another embodiment, the resulting protease mutein has an over 1000-fold increase in specificity at a site or for a particular sequence than does wild-type.

In one embodiment of this example, the specificity is measured by observing how many disparate substrate sequences a mutein protease cleaves at a given activity as compared to the number in the wild-type protease. If the mutein protease cleaves fewer substrate sequences than the wild-type, then the mutein protease has greater specificity than the wild-type. A mutein that has 10 fold higher specificity than a wild-type protease cleaves 10 fold fewer substrate sequences than the wild-type protease.

Also contemplated by the invention are libraries of protease scaffolds with various mutations that are generated and screened using methods known in the art and those detailed herein. Libraries are screened to ascertain the substrate sequence specificity of the members. Libraries of protease scaffolds are tested for specificity by exposing the members to substrate peptide sequences. The library member with the mutations that allow it to cleave the substrate sequence is identified. The protease scaffold library is constructed with enough variety of mutation in the scaffolds that any substrate peptide sequence is cleaved by a member of the library. Thus, proteases specific for any target protein can be generated.

Particular protease residues that, upon mutation, affect the activity and specificity of scaffold proteases are described here. Serine proteases are mutated and used in aspects of the invention. In one embodiment of the invention, protease muteins with altered specificity are generated by a structure-based design approach. Each protease has a series of amino acids that lines the active site pocket and make direct contact with the substrate. Throughout the chymotrypsin family, the backbone interaction between the substrate and enzyme is conserved, but the side chain interactions vary considerably. The identity of the amino acids that comprise the S1-S4 pockets of the active site determines the substrate specificity of that particular pocket. Grafting the amino acids of one serine protease to another of the same fold may modify the specificity of one to the other. Scaffold residues of serine proteases are identified using chymotrypsin numbering. For example, a mutation at position 99 in the S2 pocket to a smaller amino acid confers a preference for larger hydrophobic residues in the P2 substrate position. Using this process of selective mutagenesis, followed by substrate library screening, one can generate and identify proteases are designed with novel substrate specificities towards proteins involved with various diseases.

The amino acids of the protease that comprise the S1-S4 pockets are those that have side chains within 4 to 5 angstroms of the substrate. The interactions these amino acids have with the protease substrate are generally called “first shell” interactions because they directly contact the substrate. There are also “second shell” and “third shell” interactions that ultimately position the first shell amino acids. The invention also contemplates the mutation of those amino actions which undergo second and third shell interactions in order to change the specificity an d rate of reaction of the mutein protease of the invention.

Chymotrypsin family members share sequence and structural homology with chymotrypsin. Based on chymotrypsin numbering, the active site residues are Asp102, His57, and Ser 195. The linear amino acid sequence can be aligned with that of chymotrypsin and numbered according to the β sheets of chymotrypsin. Insertions and deletions occur in the loops between the beta sheets, but throughout the structural family, the core sheets are conserved. The serine proteases interact with a substrate in a conserved β sheet manner. Up to 6 conserved hydrogen bonds can occur between the substrate and the enzyme. All serine proteases of the chymotrypsin family have a conserved region at their N-terminus that is necessary for catalytic activity. It is generally IIGG, VVGG or IVGG (SEQ ID NOS:25, 26 and 27, respectively). Where the first amino acid in this quartet is numbered according to the chymotrypsin numbering, it is given the designation of Ile16. This numbering does not reflect the length of the precursor region.

Substrate Recognition Profiles

Serine protease substrate recognition sites are labeled according to the method of Schecter and Berger (Biochem. Biophys. Res. Commun. 27(1967) 157-162). Labels increase in number from P1, P2, . . . Pn for the substrate amino acids N-terminal to the scissile bond and P1′, P2′, . . . Pn′ for the substrate amino acids C-terminal to the scissile bond. The corresponding substrate recognition pockets on the enzyme are labeled, Sn . . . S2, S1, S1′, S2′ . . . Sn′. Thus, P2 interacts with S2, P1 with S1, P1′ with S1′, etc. Amino acids in the serine-like protease scaffold are numbered according to their alignment with the serine protease chymotrypsin. See, Blow, D. M. (1976) Acc. Chem. Res. 9, 145-152.

For serine proteases, the following amino acids in the primary sequence are determinants of specificity: 195, 102, 57 (the catalytic triad); 189, 190,191, 192, and 226 (P1); 57, and 99 (P2); 192, 217, 218 (P3), the loop between Cys168 and Cys180, 215 and 97 to 100 (P4), 41 and 151 (P2′). Position 189 in a serine protease is a residue buried at the bottom of the pocket that determines the P1 specificity. To make a variant protease with an altered substrate recognition profile, the amino acids in the three-dimensional structure that contribute to the substrate selectivity (specificity determinants) are targeted for mutagenesis.

For the serine proteases, numerous structures of family members have defined the surface residues that contribute to extended substrate specificity (Wang et al., Biochemistry 2001 Aug. 28;40(34): 10038-46; Hopfner et al., Structure Fold Des. 1999 Aug. 15;7(8): 989-96; Friedrich et al. J Biol Chem. 2002 Jan. 18;277(3): 2160-8; Waugh et al., Nat Struct Biol. 2000 September;7(9): 762-5). The family members were assayed in the conventional, negative manner wherein the investigators look for modifications in inhibitor and substrate catalysis, and not in a positive manner, wherein one looks for the alteration and possible narrowing of the specificity across all possible substrates. Among the compositions of the present invention, the I99A/N218A change to nearly exclusive Phe/Tyr specificity at P2 is undetectable by conventional (negative activity) methods.

Granzyme B

Structural determinants for granzyme B are listed in Table 10. Table 10 provides a listing of the amino acids in granzyme B determined to be of known, extended specificity. The number underneath the Cys168-Cys182 and 60's loop column headings indicate the number of amino acids in the loop between the two amino acids and in the loop. The yes/no designation under the Cys191-Cys220 column heading indicates whether the disulfide bridge is present in this protease. These regions are variable within the family of chymotrypsin-like serine proteases and represent structural determinants in themselves.

TABLE 10
Structural determinants for granzyme B.
Scaffold Residues that Determine Specificity

| | S4 | | | | | | | S2 | | | S1 | | | |
|---|---|---|---|---|---|---|---|---|---|---|---|---|---|---|
| | | | | | Cys¹⁶⁸ | S3 | | | | 60′s | | | | Cys¹⁹¹ |
| | 171 | 174 | 180 | 215 | Cys¹⁸² | 192 | 218 | 99 | 57 | loop | 189 | 190 | 226 | Cys²²⁰ |
| Granzyme B | Leu | Tyr | Glu | Tyr | 14 | Arg | Asn | Ile | His | 6 | Gly | Ser | Arg | no |

Table 11 depicts the potential target cleavage sequences for wild-type and mutein granzyme B. Residues indicated in bold are sequences that are contemplated to be differentially targeted in various individual muteins.

TABLE 11
Substrate specificities for wild-type and mutein granzyme B serine
proteases.

| | P4 | P3 | P2 | P1 |
|---|---|---|---|---|
| granzyme B | | | | |
| Native specificity | I/V | E/Q/M | P/T | D |
| VEGFR2 sequences | | | | |
| (WT) | V | L | K | D |
| (CB01) | L | V | E | D |
| (CB06) | W | F | K | D |
| | R | I | Y | D |

2A. Mutagenesis of the Granzyme B Scaffold Protease

In order to change the substrate preference of a given subsite (S1-S4) for a given amino acid, the specificity determinants that line the binding pocket are mutated, either individually or in combination. The resulting set of protease muteins, each different member having a different specificity and one or more differing mutations from one another, and the coding sequences and expression vectors producing them, constitute important aspects of the present invention. In one embodiment of the invention, a saturation mutagenesis technique is used in which the residue(s) lining the pocket is mutated to each of the 20 possible amino acids. This can be accomplished using the Kunkle method (In: Current Protocols in Molecular Biology, Ausubel et al. (eds.) John Wiley and Sons, Inc., Media Pa.). Briefly, a mutagenic oligonucleotide primer is synthesized that contains either NNS or NNK-randomization at the desired codon. The primer is annealed to the single stranded DNA template and DNA polymerase is added to synthesize the complementary strain of the template. After ligation, the double stranded DNA template is transformed into E. coli for amplification. Alternatively, single amino acid changes are made using standard, commercially available site-directed mutagenesis kits such as QuikChange (Stratagene). In another embodiment, any method commonly known in the art for site specific amino acid mutation of a protease could be used to prepare a set of protease muteins of the invention that can be screened to identify muteins that cleave VEGF, a VEGFR, or another target protein.

Granzyme B is a member of the family of chymotrypsin fold serine proteases, and has greater than 50% identity to other members of the granzyme family including granzymes C-G, cathepsin G, and rat mast cell protease II. The protein is a sandwich of two six stranded, anti-parallel β-barrel domains connected by a short α-helix. The catalytic triad is composed of Asp102, His 57 and Ser 195. The surface loops are numbered according to the additions and deletions compared to α-chymotrypsin and represent the most variable regions of this structural family. The determinants of specificity are defined by the three-dimensional structure of rat granzyme B in complex with ecotin [IEPD (SEQ ID NO: 22)], a macromolecular inhibitor with a substrate-like binding loop (Waugh et al., Nature Struct. Biol). These structural determinants of specificity include Lys 41, Ile99, Arg192, Asn218, Tyr215, Tyr174, Leu172, Arg226, and Tyr151, by chymotrypsin numbering. Interestingly, the other members of the granzyme family of serine proteases share only two of these amino acids with granzyme B. They are Tyr215 and Leu172, two residues that vary very little across the entire structural family. This suggests that while the sequence identity of the granzymes is high, their substrate specificities are very different.

To determine the role of these amino acids in extended specificity, Ile99, Arg192, Asn218 and Tyr174 were mutated to the amino acid alanine. It was determined that Ile99 contributes to P2 specificity, Asn218 and Arg192 to P3 specificity, and Tyr174 to P4 specificity. Each modified protease was profiled using a combinatorial substrate library to determine the effect of the mutation on extended specificity. Since the P1 specificity of a protease represents the majority of its specificity, the modifications do not destroy unique specificity of granzyme B towards P1 aspartic acid amino acids but modulate specificity in the extended P2 to P4 sites.

For the P3 and P4 subsites, mutations at Tyr174, Arg192 and Asn218 did not significantly affect the specificity (See Tables 7 and 8, below). Y174A increases the activity towards Leu at P4, but the rest of the amino acids continue to be poorly selected. R192A and N218A both broaden the specificity at P3. Instead of a strong preference for glutamic acid, Ala, Ser, Glu and Gln are similarly preferred in the mutant. The overall activity (k_{cat}/Kₘ) of the mutant is less than 10% below the wild-type activity toward an ideal wild-type substrate, N-acetyl-Ile-Glu-Pro-Asp-AMC (7-amino-4-methylcoumarin) (Ac-IEPD-AMC) (SEQ ID NO: 22).

A much more dramatic effect is observed at the P2 subsite (See Table 7, below). In wild-type granzyme B, the preference is broad with a slight preference for Pro residues. I99A narrows the P2 specificity to Phe and Tyr residues. Phe is now preferred nearly 5 times over the average activity of other amino acids at this position. Within the chymotrypsin family of serine proteases, more than a dozen proteases have a small residue at this structural site, either an asparagine, serine, threonine, alanine or glycine. From this group, two proteases have been profiled using combinatorial substrate libraries, (plasma kallikrein and plasmin), and both show strong preferences towards Phe and Tyr. These two results suggest that any serine protease that is mutated to an Asn, Ser, Thr, Gly or Ala at position 99 will show the same hydrophobic specificity found in plasma kallikrein, plasmin and the I99A granzyme B mutant.

The understanding of the P2 specificity determinants may be expanded to the contrasting mutation and substrate preference. Nearly two dozen chymotrypsin-fold serine proteases have an aromatic amino acid at position 99. Four of these proteases have been profiled using combinatorial substrate libraries: human granzyme B, tissue type plasminogen activator, urokinase type plasminogen activator, and membrane type serine protease 1. All but granzyme B have a preference for serine, glycine and alanine amino acids at the substrate P2 position. In one embodiment, a mutein from Tables 12A, 12B and 13 activates the substrate by cleavage at the natural granzyme B recognition site. In another embodiment, a mutein cleaves granzyme B at a sequence other than the natural site and inactivates it. Also, the muteins described herein are on the rat granzyme B scaffold (RNKP1), but the numbering and residues also apply to the human scaffold. Both human and rat will be made in the expression system of the invention.

TABLE 12A
Granzyme B Mutations

| | A. | | | | | | | | | | | | | |
|---|---|---|---|---|---|---|---|---|---|---|---|---|---|---|
| | S4 | | | | | | | S2 | | | S1 | | | |
| | | | | | Cys | S3 | | | | 60′s | | | | Cys |
| Mutant | 171 | 174 | 180 | 215 | 168-182 | 192 | 218 | 99 | 57 | Loop | 189 | 190 | 226 | 191-220 |
| Wild- | Leu | Tyr | Glu | Tyr | 14 | Arg | Asn | Ile | His | 6 | Gly | Ser | Arg | No |
| type | | | | | | | | | | | | | | |
| I99F | Leu | Tyr | Glu | Tyr | 14 | Arg | Asn | Phe | His | 6 | Gly | Ser | Arg | No |
| I99A | Leu | Tyr | Glu | Tyr | 14 | Arg | Asn | Ala | His | 6 | Gly | Ser | Arg | No |
| I99K | Leu | Tyr | Glu | Tyr | 14 | Arg | Asn | Lys | His | 6 | Gly | Ser | Arg | No |
| N218A | Leu | Tyr | Glu | Tyr | 14 | Arg | Ala | Ile | His | 6 | Gly | Ser | Arg | No |
| N218T | Leu | Tyr | Glu | Tyr | 14 | Arg | Thr | Ile | His | 6 | Gly | Ser | Arg | No |
| N218V | Leu | Tyr | Glu | Tyr | 14 | Arg | Val | Ile | His | 6 | Gly | Ser | Arg | No |
| R192A | Leu | Tyr | Glu | Tyr | 14 | Ala | Asn | Ile | His | 6 | Gly | Ser | Arg | No |
| R192E | Leu | Tyr | Glu | Tyr | 14 | Glu | Asn | Ile | His | 6 | Gly | Ser | Arg | No |
| Y174A | Leu | Ala | Glu | Tyr | 14 | Arg | Asn | Ile | His | 6 | Gly | Ser | Arg | No |
| Y174V | Leu | Val | Glu | Tyr | 14 | Arg | Asn | Ile | His | 6 | Gly | Ser | Arg | No |
| R192A/ | Leu | Tyr | Glu | Tyr | 14 | Ala | Ala | Ile | His | 6 | Gly | Ser | Arg | No |
| N218A | | | | | | | | | | | | | | |
| R192E/ | Leu | Tyr | Glu | Tyr | 14 | Glu | Ala | Ile | His | 6 | Gly | Ser | Arg | No |
| N218A | | | | | | | | | | | | | | |

TABLE 12B
Granzyme B Mutations - by CB numbering.

| | B. | | | | | | | | | | | | | |
|---|---|---|---|---|---|---|---|---|---|---|---|---|---|---|
| | S4 | | | | | S3 | | S2 | | | S1 | | | |
| CB01 = | Leu | Tyr | Glu | Tyr | 14 | Arg | Ala | Ala | His | 6 | Gly | Ser | Arg | No |
| I99A/ | | | | | | | | | | | | | | |
| N218A | | | | | | | | | | | | | | |
| I99A/ | Leu | Tyr | Glu | Tyr | 14, | Arg | Ala | Ala | His | 6 | Gly | Ser | Arg | No |
| L171D/ | | | | | Asp | | | | | | | | | |
| N218A | | | | | | | | | | | | | | |
| CB06 = | | Glu | | | | | Ala | Ala | | | | | | |
| I99A/ | | | | | | | | | | | | | | |
| Y174E/ | | | | | | | | | | | | | | |
| N218A | | | | | | | | | | | | | | |
| CB02 = | Glu | | | | | | Ala | Ala | | | | | | |
| I99A/ | | | | | | | | | | | | | | |
| L171E/ | | | | | | | | | | | | | | |
| N218A | | | | | | | | | | | | | | |
| CB05 = | | | | | | | Ala | Ala | | | | | | |
| I99A/ | | | | | | | | | | | | | | |
| K172D/ | | | | | | | | | | | | | | |
| N218A | | | | | | | | | | | | | | |
| CB10 = | | | | | | Met | Ala | Ala | | | | | | |
| I99A/ | | | | | | | | | | | | | | |
| R192M/ | | | | | | | | | | | | | | |
| N218A | | | | | | | | | | | | | | |
| CB07 = | | Asp | | | | | Ala | Ala | | | | | | |
| I99A/ | | | | | | | | | | | | | | |
| Y174D/ | | | | | | | | | | | | | | |
| N218A | | | | | | | | | | | | | | |

TABLE 13
Specificity Profiles of Granzyme B Muteins

| Mutant | P4 | P3 | P2 | P1 |
|---|---|---|---|---|
| Wild-type | Ile/Val | Glu | X | Asp |
| I99F | Ile/Val | Glu | X | Asp |
| I99A | Ile/Val | Glu | Phe | Asp |
| I99R | Ile/Val | Glu | Phe/Pro | Asp |
| N218A | Ile/Val | X | X | Asp |
| N218T | Ile/Val | Ala/Ser | X | Asp |
| N218V | Ile/Val | X | X | Asp |
| R192A | Ile/Val | Glu | X | Asp |
| R192E | Ile/Val | Lys/Gln/Ser | X | Asp |
| Y174A | Ile/Val/Leu | Glu | X | Asp |
| Y174V | Ile/Val | Glu | X | Asp |
| I99A/N218A | Phe/Leu/Ile/Val | Ala/Ser | Phe | Asp |
| R192A/N218A | Ile/Val | Ala/Gln/Ser | X | Asp |
| R192E/N218A | Ile/Val | Arg/Lys/Ala | X | Asp |

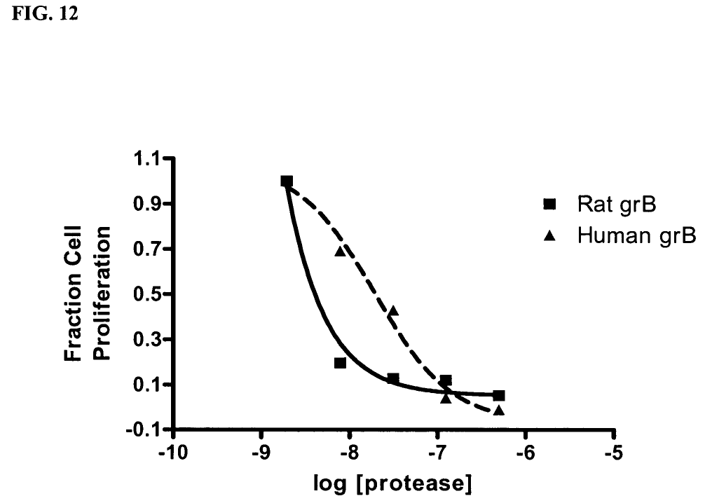

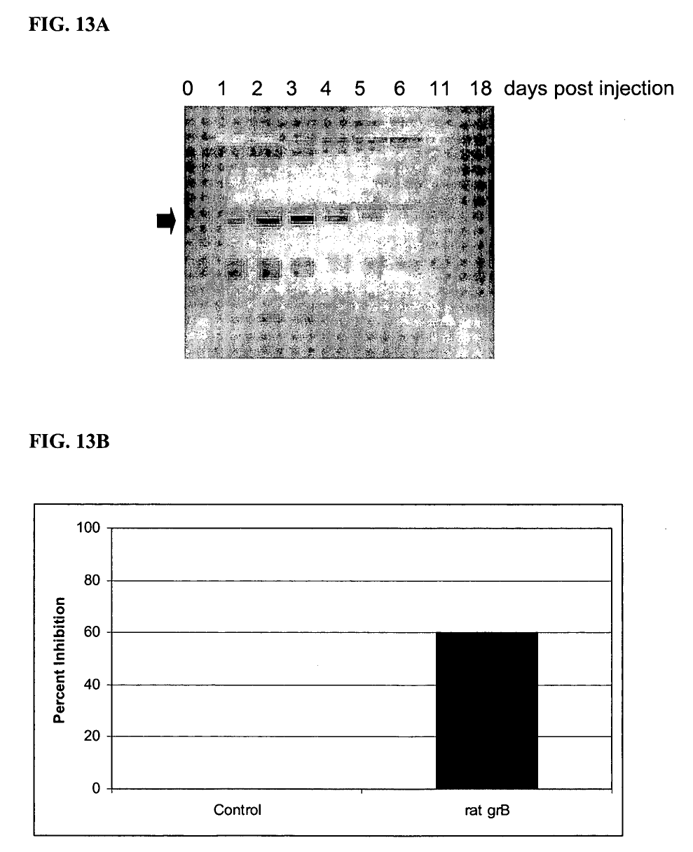

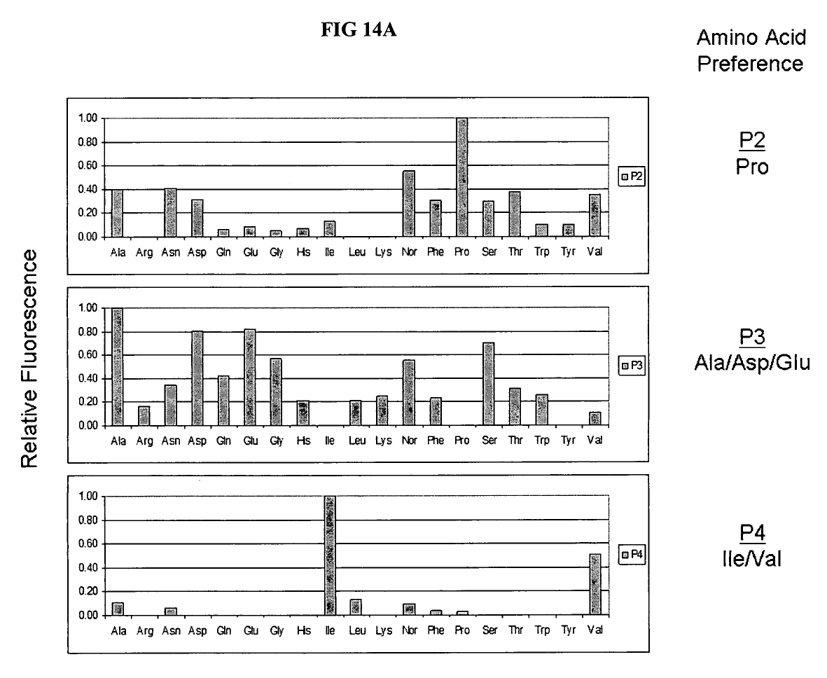

Specific compositions provided in FIGS. 12A-12B and Example 4 are as shown in Table 14.

TABLE 14

| Mutant | P4 | P3 | P2 | P1 |
|---|---|---|---|---|
| CB01 = I99A/ | Pro/Leu/ | X | Phe | Asp |
| N218A | Phe/Ile | | | |
| CB06 = I99A/ | Trp | Ala/Glu/Ser/X | Phe/Tyr | Asp |
| N218A/ | | | | |
| Y174E | | | | |
| CB02 = I99A/ | Trp | X | Phe/Tyr | Asp |
| N218A/ | | | | |
| L171E | | | | |
| CB05 = I99A/ | Ile/Val/Pro | Glu/Ser | Phe/Tyr | Asp |
| N218A/ | | | | |
| K172D | | | | |
| CB10 = I99A/ | Ile > Leu/Nor | Gln/Ala | Phe/Tyr | Asp |
| N218A/ | | | | |
| R192M | | | | |
| CB07 = I99A/ | Trp | X | Phe/Tyr | |
| N218A/ | | | | |
| Y174D | | | | |

From Tables 12 and 13, the determinants of specificity selected to be altered in rat granzyme B are provided in Table 15 below.

In order to develop novel strategies to attenuate VEGF signaling for angiogenesis therapy, granzyme B polypeptides are engineered to selectively cleave and inactivate VEGF receptor 2 (KDR). Wild-type granzyme B protease domain (herein referred to as granzyme B) and mutants thereof are cloned, expressed, purified, and profiled by PSSCL. See, PCT publication WO 01/94332, incorporated by reference herein in its entirety. Wild-type and mutein granzyme B are then assayed for the cleavage of purified VEGF receptor, as further described in the Examples.

Granzyme B variants that are able to cleave the purified VEGF receptor are assayed for the cleavage of the receptor on endothelial cells, wherein cleavage results in abrogation of cell proliferation resulting from VEGF signaling. See, e.g. Yilmaz et al., 2003 Biochem. Biophys. Res. Commun. 306(3): 730-736; Gerber et al., 1998 J Biol Chem. 273(46): 30336-43. Promising variants are then tested in animal models angiogenesis and tumor growth, including the mouse micropocket corneal assay and tumor xenographs. See, e.g. Kuo et al., PNAS, 2001, 98:4605-4610.

The determinants of specificity selected to be altered in rat granzyme B are shown in Table 15. Mutants of granzyme B were made by QuikChange PCR (Stratagene) according to manufacturer's protocol. Table 15 is a non-limiting listing of a variety of resulting mutant granzyme B polypeptides (muteins), wherein one or more residues in the wild-type granzyme B scaffold are replaced with a residue other than the one at that position in wild-type granzyme B. The granzyme B wild-type residues, identified using chymotrypsin numbering, are provided in the left column and the exemplary granzyme B mutant residues are provided in the right column. Note that wild-type Leu171A designates a one amino acid insertion as compared to chymotrypsin.

TABLE 15
Granzyme B mutein constructs

| A. wild-type granzyme | |
|---|---|
| B residue (chymotrypsin | |
| numbering) by CB # | Replacement Mutein residue |
| CB92 | L171A |
| CB93 | L171A/I99A/N218A |
| CB94 | L171D |
| CB95 | L171D/I99A/N218A |
| CB96 | L171E/I99A/N218A/Y174E |
| CB97 | L171N/I99A/N218A/Y174E |
| CB98 | L171W |
| CB99 | L171W/I99A/N218A |
| CB100 | L171E/K172E/I99A/N218A/Y174E |
| CB101 | Y215A |
| CB102 | Y215A/I99A/N218A |
| CB103 | Y215D |
| CB104 | Y215D/I99A/N218A |
| CB105 | Y215F |
| CB106 | Y215F/I99A/N218A |
| CB107 | Y174W |
| CB108 | Y174W/I99A/N218A |
| CB109 | K172E/I99A/N218A/Y174E |
| CB110 | R192Y |
| CB111 | R192Y/I99A/N218A |
| CB112 | R192Y/I99A/N218A/Y174E |
| CB113 | N218D |
| CB114 | N218D/I99A |
| CB115 | N218D/I99A/Y174E |
| CB116 | N218E |
| CB117 | N218E/I99A |
| CB118 | N218E/I99A/Y174E |
| CB119 | K97E/I99A |
| CB120 | K97E/I99A/N218A |
| CB121 | K97E/I99A/N218A/Y174E |
| CB122 | K97Q |
| CB123 | K97Q/I99A/N218A |
| CB124 | K97Q/I99A/N218A/Y174E |
| CB125 | K97Y/I99A |
| CB126 | K97Y/I99A/N218A |
| CB127 | K97Y/I99A/N218A/Y174E |
| CB128 | I99D |
| CB129 | I99D/N218A |
| CB130 | I99D/N218A/Y174E |
| CB131 | I99E |
| CB132 | I99E/N218A |
| CB133 | I99E/N218A/Y174E |
| CB134 | I99Q |
| CB135 | I99Q/N218A |
| CB136 | I99Q/N218A/Y174E |
| CB137 | I99Y |
| CB138 | I99Y/N218A |
| CB139 | I99Y/N218A/Y174E |
| CB140 | K41A |
| CB141 | K41A/I99A/N218A |
| CB142 | K41A/I99A/N218A/Y174E |
| CB143 | Y151A |
| CB144 | Y151A/I99A/N218A |
| CB145 | Y151A/I99A/N218A/Y174E |
| CB146 | R192Y/I99A/N218D/Y174E |
| CB147 | K97Q/I99A |
| CB148 | Y215N/I99A/N218A |
| CB149 | Y215N/N218A |
| CB150 | Y174A/I99A/N218A |
| B. Granzyme B | |
| wild-type amino acid | Contemplated muteins |
| Ile 99 | Ala, Tyr, Asp, Glu |
| Asn218 | Asp |
| Tyr174 | Ala, Glu, Asp, Trp, Gln |
| Leu171 | Ala, Glu, Gln, Asn, Trp |
| Lys172 | Ala, Glu |
| Lys41 | Ala |
| Tyr 151 | Ala |
| Arg192 | Tyr |
| Lys97 | Ala, Tyr, Glu, Gln, |
| Tyr215 | Ala, Phe, Trp Ala, Glu |

A mutated granzyme B polypeptide (“mutein”) may contain a single mutation per polypeptide, or may contain two or more mutated residues in any combination provided in Table 9. In one embodiment, an Ile residue at position 99 is replaced with an Ala residue, wherein the mutein is designated as I99A. Likewise, other exemplary muteins with a single residue changed include R192A, N218A. Exemplary muteins with two residues changed include I99A/N218A, R192A/N218A and R192E/N218A. Other nonlimiting muteins containing one, two, three, four, five or more replaced residues are provided herein.

2B. Mutagenesis of the MT-SP1 Scaffold Protease

In order to change the substrate preference of a given subsite (S1-S4) for a given amino acid, the specificity determinants that line the binding pocket are mutated, either individually or in combination. In one embodiment of the invention, a saturation mutagenesis technique is used in which the residue(s) lining the pocket is mutated to each of the 20 possible amino acids. This can be accomplished using the Kunkle method (In: Current Protocols in Molecular Biology, Ausubel et al. (eds.) John Wiley and Sons, Inc., Media Pa.). Briefly, a mutagenic oligonucleotide primer is synthesized that contains either NNS or NNK-randomization at the desired codon. The primer is annealed to the single stranded DNA template and DNA polymerase is added to synthesize the complementary strain of the template. After ligation, the double stranded DNA template is transformed into E. coli for amplification. Alternatively, single amino acid changes are made using standard, commercially available site-directed mutagenesis kits such as QuikChange (Stratagene). In another embodiment, any method commonly known in the art for site specific amino acid mutation of MT-SP1 could be used.

MT-SP1 is a mosaic protein containing a transmembrane domain, two CUB domains, four LDLR repeats, and a serine protease domain. The protease domain of MT-SP1 has been expressed in bacteria or yeast in milligram quantities and purified. Profiling by positional scanning substrate combinatorial libraries (PSSCL) revealed that it has trypsin-like activity, demonstrating a strong preference for basic residues at the P1 position. The extended P2-P4 specificity of MT-SP1 is shown in Table 16.

TABLE 16
Extended P2-P4 Specificity of Wild-type MT-SP1

| P4 | P3 | P2 | P1 |
|---|---|---|---|
| Arg/Lys | Xxx | Xxx | Arg/Lys or |
| Xxx | Arg/Lys | Xxx | Arg/Lys |

wherein Xxx is any amino acid.

Thus MT-SP1 appears to have a specificity switch, wherein it accepts a positively charged residue in the P4 position or a positively charged residue in the P3 position. The crystal structure of the protease domain of MT-SP1 has been solved, providing a structural rationale for its substrate specificity profile.

To develop novel muteins useful for attenuating VEGF signaling for anti-angiogenesis therapy, MT-SP1 polypeptides are engineered to selectively cleave and inactivate VEGF receptor 2 (KDR) selectively. Wild-type MT-SP1 protease domain (herein referred to as MT-SP1) and mutants thereof are cloned, expressed, purified, and profiled by PSSCL. See, PCT publication WO 01/94332, incorporated by reference herein in its entirety. Wild-type and mutant MT-SP1 are then assayed for the cleavage of purified VEGF receptor, as further described and illustrated in the Examples below.

MT-SP1 variants that are able to cleave the purified VEGF receptor are assayed for the cleavage of the receptor on endothelial cells, wherein cleavage results in abrogation of cell proliferation resulting from VEGF signaling. See, e.g. Yilmaz et al., 2003 Biochem. Biophys. Res. Commun. 306(3): 730-736; Gerber et al., 1998 J Biol Chem. 273(46): 30336-43. Promising variants are then tested in animal models angiogenesis and tumor growth, including the mouse micropocket corneal assay and tumor xenografts. See, e.g. Kuo et al., PNAS, 2001, 98:4605-4610.

Mutants of MT-SP1 were made by QuikChange PCR (Stratagene) according to the manufacturer's protocol. A non-limiting listing of a variety of resulting mutant MT-SP1 polypeptides (muteins) is provided in Table 17, and their corresponding CB numbers are provided in Table 18. The MT-SP1 wild-type residues, identified using chymotrypsin numbering, are provided in the left column and the MT-SP1 mutants are provided in the right column. Asp60b and Arg60c are part of an insertion in MT-SP1 not present in chymotrypsin. Therefore all the residues in this loop are assigned to residue 60 when using chymotrypsin numbering.

TABLE 17
MT-SP1 mutein constructs

| wild-type MT-SP1 residue | |
|---|---|
| (chymotrypsin numbering) | Replacement Mutein residue |
| Asp60b | Ala, Arg, Ile, Phe |
| Arg60c | Ala, Asp, Ile, Phe, Trp |
| Phe97 | Ala, Arg, Asn, Asp, Glu, Trp |
| Phe99 | Ala, Arg, Asn, Asp, Glu, Tyr, Trp, Val |
| Tyr146 | Ala, Arg, Asn, Asp, Glu, Phe, Trp |
| Leu172 | Ala, Arg, Asn, Asp, Glu, Phe |
| Gln175 | Ala, Arg, Asp, Glu, Phe, Val |
| Met180 | Ala, Arg, Glu, Tyr |
| Gln192 | Ala, Arg, Asp, Phe, Val |
| Trp215 | Arg, Asp, Ile, Phe, Tyr |
| Asp217 | Ala, Arg, Glu, Phe, Val |
| Lys224 | Ala, Asp, Phe, Val |

TABLE 18
MT-SP1 muteins labeled by CB number

| | |
|---|---|
| CB0011 | F97N |
| CB0012 | F97D |
| CB0013 | F97E |
| CB0014 | F99Y |
| CB0015 | F99W |
| CB0016 | Y146F |
| CB0017 | L172N |
| CB0018 | L172D |
| CB0019 | L172E |
| CB0020 | Q175D |
| CB0021 | Q175E |
| CB0022 | D217A |
| CB0023 | D217V |
| CB0024 | D217F |
| CB0031 | F97A |
| CB0032 | F97W |
| CB0033 | F97R |
| CB0034 | F99N |
| CB0035 | F99D |
| CB0036 | F99E |
| CB0037 | F99A |
| CB0038 | F99V |
| CB0039 | F99R |
| CB0040 | Y146N |
| CB0041 | Y146D |
| CB0042 | Y146E |
| CB0043 | Y146A |
| CB0044 | Y146W |
| CB0045 | Y146R |
| CB0046 | L172A |
| CB0047 | L172V |
| CB0048 | L172F |
| CB0049 | L172R |
| CB0050 | Q175A |
| CB0051 | Q175V |
| CB0052 | Q175F |
| CB0053 | Q175R |
| CB0054 | D217E |
| CB0055 | D217R |
| CB0056 | W215F |
| CB0057 | W215Y |
| CB0058 | W215I |
| CB0059 | W215D |
| CB0060 | W215R |
| CB0061 | Q192A |
| CB0062 | Q192V |
| CB0063 | Q192D |
| CB0064 | Q192R |
| CB0065 | Q192F |
| CB0066 | K224A |
| CB0067 | K224F |
| CB0068 | K224V |
| CB0069 | K224D |
| CB0070 | M180E |
| CB0071 | M180Y |
| CB0072 | M180R |
| CB0073 | M180A |
| CB0074 | D60bI |
| CB0075 | D60bF |
| CB0076 | D60bR |
| CB0077 | D60bA |
| CB0078 | R60cI |
| CB0079 | R60cF |
| CB0080 | R60cD |
| CB0081 | R60cA |
| CB0082 | R60cW |
| CB0083 | L172D/Q175D |
| CB0150 | F99V/L172D |
| CB0151 | F99V/L172D/Q175D |
| CB0152 | F99V/K224F |
| CB0153 | F99V/M180E |
| CB0154 | F99V/Y146D |
| CB0155 | Y146D/K224F |
| CB0156 | Y146D/M180E |
| CB0157 | Y146D/L172D/Q175D |
| CB0158 | F99V/Y146D/L172D/Q175D |
| CB0159 | F99I/L172D/Q175D |
| CB0160 | F99L/L172D/Q175D |
| CB0161 | F99T/L172D/Q175D |
| CB0162 | F99A/L172D/Q175D |
| CB0173 | F99I/K224F |
| CB0174 | F99L/K224F |
| CB0175 | F99T/K224F |
| CB0176 | F99V/Y146D/K224F |
| CB0177 | F99I/Y146D/K224F |
| CB0178 | F99L/Y146D/K224F |
| CB0179 | F99T/Y146D/K224F |

In Table 18, mutations are identified using the chymotrypsin numbering system. Thus, W215Y means that a tryptophan at position 215 of MT-SP1 according to the chymotrypsin numbering system is changed to a tyrosine at that position.

In any given embodiment, a mutated MT-SP1 polypeptide (“mutein”) may contain a single mutation per polypeptide, or may contain two or more mutated residues per polypeptide in any combination. Exemplary replacements of wild-type residues are provided in Table 18. In one exemplary embodiment, a Leu residue at position 172 is replaced with an Asp residue, wherein the mutein is designated as L172D. In another exemplary embodiment, an Asp60b residue is replaced by any one of Ala, Arg, Ile or Phe. In a further exemplary embodiment a variant MT-SP1 includes at least one of Y146F, L172D, N175D and D217F, and may contain two, three, four or more such residue replacements.

3. Expression and Purification of Variant Protease

In one embodiment, the protease is expressed in an active form. In another embodiment, the protease is expressed in an inactive, zymogen form. In one embodiment, the protease is expressed by a heterologously expression system such as an E. coli, Pichia pastoris, S. cerevisiae, or a baculovirus expression system. In a preferred embodiment, the protease is expressed in a mammalian cell culture expression system. Exemplary mammalian cell cultures are derived from rat, mouse, or preferably human cells. The protein can either be expressed in an intracellular environment or excreted (secreted) into the media. The protease can also be expressed in an in vitro expression system.

To purify the variant protease, column chromatography can be used. The protease may be engineered to contain a C-terminal 6-His tag for purification on a Nickel column. Depending on the pI of the protease, a cation or anion exchange column can be used in the purification method for the protease. The protease can be stored in a low pH buffer that minimizes its catalytic activity so that it will not degrade itself. Purification can also be accomplished through immunoabsorption, gel filtration, or any other purification method commonly used in the art. The protease can be stored in a low pH buffer that minimizes its catalytic activity so that it will not degrade itself.

4. Synthesis of ACC Positional Scanning Libraries

Those of skill in the art will recognize that many methods can be used to prepare the peptides and the libraries of the invention. Suitable embodiments are further illustrated in the Examples.

5. Determination of Specificity Changes for Protease Muteins.

Essential amino acids in the proteases generated using the methods of the present invention are identified according to procedures known in the art, such as site-directed mutagenesis or saturation mutagenesis of active site residues or disclosed herein. In one technique, residues that form the S1-S4 pockets that have been shown to be important determinants of specificity are mutated to every possible amino acid, either alone or in combination. See, e.g., Legendre, et al., JMB (2000) 296: 87-102. Substrate specificities of the resulting mutants will be determined using the ACC positional scanning libraries and by single substrate kinetic assays. See, e.g., Harris, et al. PNAS, 2000, 97:7754-7759.

Multiple amino acid substitutions are made and tested using known methods of mutagenesis and screening, such as those disclosed herein or already known in the art. See, e.g., Reidhaar-Olson and Sauer 1988 Science 241:53-57, or Bowie and Sauer 1989 Proc. Natl. Acad. Sci. USA 86:2152-2156. Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display based methods (e.g., Legendre et al., JMB, 2000: 296:87-102; Lowman et al., Biochem. 30:10832-10837, 1991; Ladner et al., U.S. Pat. No. 5,223,409; Huse, PCT Publication WO 92/06204) and region-directed mutagenesis (Derbyshire et al., Gene 46:145, 1986; Ner et al., DNA 7:127, 1988).

Mutagenesis methods as disclosed above can be combined with high-throughput, automated screening methods to detect activity of cloned, mutagenized polypeptides in host cells. Mutagenized DNA molecules that encode proteolytically active proteins or precursors thereof are recovered from the host cells and rapidly sequenced using modern equipment. These methods allow the rapid determination of the importance of individual amino acid residues in a polypeptide of interest, and can be applied to polypeptides of unknown structure.

In one embodiment protease phage display is used to screen the libraries of mutant proteases of the invention for various affinities to specific substrate sequences as described in the art. See, e.g., Legendre et al., JMB, 2000: 296:87-102, and Corey et al., Gene, 1993 Jun. 15;128(1):129-34.

The invention also provides methods for detecting and quantitating an enzymatically active protease of the invention. The method includes: (a) contacting the protease with a library of peptides of the invention in such a manner whereby the fluorogenic moiety is released from the peptide sequence, thereby forming a fluorescent moiety; (b) detecting the fluorescent moiety; and (c) determining the sequence of the peptide sequence, thereby determining the peptide sequence specificity profile of the protease.

In a preferred embodiment of the above-described method, the method further includes, (d) quantifying the fluorescent moiety, thereby quantifying the protease.

Moreover, in each of the aspects and embodiments set forth hereinabove, the protease can be substantially any protease of interest, but is preferably aspartic protease, cysteine protease, metalloprotease or serine protease. The protease assayed using a method of the invention can be derived from substantially any organism, including, but not limited to, mammals (e.g. humans), birds, reptiles, insects, plants, fungi and the like. In a preferred embodiment, the protease is derived from a microorganism, including, but not limited to, bacteria, flugi, yeast, viruses, and protozoa.

The methods, illustrated in Example 3 can be repeated iteratively or in parallel to characterize a variant protease that has the desired specificity and selectivity at each of the extended binding subsites, P2, P3, and P4. In some cases, mutations in serine proteases have shown that each of the subsites that form the active site (S1-S4) function independently of one another, such that modification of specificity at one subsite has little influence on specificity at adjacent subsites. Thus, engineering substrate specificity and selectivity throughout the extended binding site can be accomplished in a step-wise manner.

Mutant proteases that match the desired specificity profiles, as determined by substrate libraries, are then assayed using individual peptide substrates corresponding to the desired cleavage sequence. Variant proteases are also assayed to ascertain that they will cleave the desired sequence when presented in the context of the full-length protein. The activity of the target protein is also assayed to verify that its function has been destroyed by the cleavage event. The cleavage event is monitored by SDS-PAGE after incubating the purified full-length protein with the variant protease. In another embodiment, mutant proteases are combined to acquire the specificity of multiple proteases. A mutation at one residue of a scaffold, which produces specificity at one site, is combined in the same protease with another mutation at another site on the scaffold to make a combined specificity protease.

Any number of mutations at discrete sites on the same scaffold can be used to create a combined specificity protease. In one embodiment, the granzyme B scaffold comprises a polypeptide 95% identical to the amino acid sequence of wild-type granzyme B of SEQ ID NO:1, and the polypeptide has at least one mutation at one or more of the positions 41, 57, 58, 59, 60, 61, 62, 63, 97, 98, 99, 100, 102, 151, 169, 170, 171, 171A, 172, 173, 174, 175, 176, 177, 178, 179, 180, 181, 189, 190, 191, 192, 195, 215, 217 or 218, wherein the numbering is for chymotrypsin. In a specific embodiment, the granzyme B mutein is the CB06 construct comprising I99A/N218A/Y174E. In different embodiments, the mutein is a CB01 construct comprising I99A/N218A (which cleaves both caspase and VEGFR), the mutein is a CB02 comprising I99A/N218A/L171E, the mutein is a CB05 construct comprising I99A/N218A/K172D, or the mutein is a CB10 construct comprising I99A/N218A/R192M. In a mutein granzyme B embodiment, at least one residue is replaced as compared to the granzyme B wild-type polypeptide sequence of SEQ ID NO:20 (human) or SEQ ID NO:21 (rat). Further nonlimiting contemplated granzyme B muteins are provided in Tables 12 and 13.

In another embodiment, the MT-SP1 scaffold comprises a polypeptide 95% identical to the amino acid sequence of wild-type MT-S P1 protease domain of SEQ ID NO:23, and the polypeptide has at least one mutation at one or more of the positions 171, 174, 180, 215, 192, 218, 99, 57, 189, 190, 226, 146, 172, 175, 41, 58, 59, 60, 61, 62, 63, 97, 98, 100, 102, 151, 169, 170, 171A, 173, 176, 177, 178, 179, 181, 191, 195 or 224 or 217, wherein the numbering is for chymotrypsin.

These sites belong to the following S pockets:

S1′: 146, 151,

S1: 189, 190, 226, 191, 195

S2: 99, 41, 57, 58, 59, 60, 61, 62, 63, 97, 98, 100, 102

S3: 192, 218, 146

S4: 171, 174, 179, 180, 215, 99, 172, 175, 97. 98, 169, 170, 171A, 173, 176, 177, 178, 181, 224, 217

In an exemplary embodiment, the mutein is L172D comprising leucine replaced with aspartic acid at position 172. In another embodiment, the mutein is Y146F comprising tyrosine replaced with phenylalanine at position 146. In another embodiment, the mutein is N175D comprising asparagine replaced with aspartic acid at position 175. In yet another embodiment, the mutein is D217F comprising aspartic acid replaced with phenylalanine at position 217. In a mutein MT-SP1 embodiment, at least one residue is replaced as compared to the MT-SP1 wild-type polypeptide sequence of SEQ ID NO:1. Further nonlimiting contemplated MT-SP1 muteins are provided herein.

Proteins targeted for cleavage and inactivation can be identified by the following criteria: 1) the protein is involved in pathology; 2) there is strong evidence the protein is the critical point of intervention for treating the pathology; 3) proteolytic cleavage of the protein will likely destroy its function. By these criteria, VEGF and the VEGFRs are excellent targets for protease-mediated therapies of the invention. Cleavage sites within target proteins are identified by the following criteria: 1) they are located on the exposed surface of the protein; 2) they are located in regions that are devoid of secondary structure (i.e. not in β sheets or α helices), as determined by atomic structure or structure prediction algorithms (these regions tend to be loops on the surface of proteins or stalks on cell surface receptors); or 3) they are located at sites that are likely to inactivate the protein, based on its known function. Cleavage sequences are e.g., four residues in length to match the extended substrate specificity of many serine proteases, but can be longer or shorter.

In one embodiment of the invention, target protein-assisted catalysis is used to generate proteases specific for a target VEGF or VEGFR protein. A single mutation in the substrate sequence binding site of the protease can alter its specificity and cause it to have a change in substrate sequence specificity. Thus, substrate sequence specificity can be altered using a small number of mutations.

Using the methods disclosed above, one of ordinary skill in the art can identify and/or prepare a variety of polypeptides that are substantially homologous to a protease scaffold or allelic variants thereof and retain the proteolysis activity of the wild-type protein scaffold but vary from it in specificity. In one embodiment, these polypeptides are based on the scaffold amino acid sequences of granzyme B or MT-SP1. Such polypeptides may optionally include a targeting moiety comprising additional amino acid residues that form an independently folding binding domain. Such domains include, for example, an extracellular ligand-binding domain (e.g., one or more fibronectin type III domains) of a cytokine receptor; immunoglobulin domains; DNA binding domains (see, e.g., He et al., Nature 378:92-96, 1995); affinity tags; and the like. Such polypeptides may also include additional polypeptide segments as generally disclosed above.

Protease Polypeptides

The protease muteins and protease libraries of the invention include a polypeptide having an amino acid sequence of one or more of the proteases whose sequence is provided in any one of the scaffolds described herein. The invention also provides a mutant or variant protease any of whose residues may be changed from the corresponding residues shown in any one of the scaffolds described herein, while still encoding a protein that maintains its protease activities and physiological functions, or a functional fragment thereof. In a preferred embodiment, the mutations occur in the S1-S4 regions of the protease as detailed herein.

In general, a protease variant that preserves protease-like function includes any variant in which residues at a particular position in the sequence have been substituted by other amino acids, and further include variants produced by, relative to the wild-type or parent protein sequence, inserting an additional residue or residues between two residues of the parent protein as well as by deleting one or more residues from the parent sequence. Any amino acid substitution, insertion, or deletion is contemplated by the methods, muteins, and mutein libraries of the invention. In favorable circumstances, the substitution is a conservative substitution as described above.

One aspect of the invention pertains to isolated proteases, and biologically-active portions thereof, as well as derivatives, fragments, analogs or homologs thereof. Also provided are polypeptide fragments suitable for use as immunogens to raise anti-protease antibodies. In one embodiment, proteases of the invention are produced by recombinant DNA techniques. As an alternative to recombinant expression, a protease protein or polypeptide can be synthesized chemically using standard peptide synthesis techniques as described above.

Biologically-active portions of protease proteins include peptides comprising amino acid sequences sufficiently homologous to or derived from the amino acid sequences of the full length protease proteins, but with fewer amino acids than the full-length protease proteins, and that exhibit at least one activity of the full length protease protein. Typically, biologically-active portions comprise a domain or motif with at least one activity of the protease protein. A biologically-active portion of a protease protein is a polypeptide which is, for example, 10, 25, 50, 60, 70, 80, 90, 100, 110, 120, 130, 140, 150, 160, 170, 180, 190, 200, 210, 220, 230, 240, 241, 242, 243, 244, 245 or 246 amino acid residues in length, wherein wild-type full length granzyme B is considered to be 247 amino acids in length. Alternatively, a biologically-active portion of a protease protein is a polypeptide which is, for example, 10, 25, 50, 60, 70, 80, 90, 100, 110, 120, 130, 140, 150, 160, 170, 180, 190, 200, 210, 220, 230, 240, 250, 260, 270, 280, 290, 300 or more amino acid residues in length, and increasing in amino acid length in whole integers of one (1), up to a length of 855 amino acids, wherein wild-type full length MT-SP1 is considered to be 855 amino acids in length (SEQ ID NO:1), and mature is less than 855 aa in length. In general, a “fragment” or a “portion” of a polypeptide contains at least one less amino acid residues than the full length polypeptide. The one or more deleted amino acids may be removed from the N-terminus, the C-terminus, or an internal portion.

Moreover, other biologically-active portions of a protein, from which other regions of the protein have been deleted, can be prepared by recombinant techniques and evaluated for one or more of the functional activities of a native protease.

In one embodiment, the protease has an amino acid sequence of one of the scaffolds described herein or one of the mutants of the scaffolds. Thus, the protease protein is substantially homologous to one of the scaffolds described herein or one of its muteins, and retains the functional activity of the scaffold protein, yet differs in amino acid sequence due to natural allelic variation or mutagenesis and may differ in specificity as described herein. Representative muteins are disclosed in Tables 12A, 12B, 15, 17 and 18 herein.

Determining Homology between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid or amino acid sequence homology may be determined as the degree of identity between two sequences. The homology may be determined using computer programs known in the art, such as GAP software provided in the GCG program package. See, Needleman and Wunsch, 1970. J Mol Bio 148: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides protease chimeric or fusion proteins. As used herein, a protease “chimeric protein” or “fusion protein” comprises a protease polypeptide operatively-linked to a non-protease polypeptide. A “protease polypeptide” refers to a polypeptide having an amino acid sequence corresponding to one of the scaffolds described herein or one of the muteins of the scaffolds, whereas a “non-protease polypeptide” refers to a polypeptide having an amino acid sequence corresponding to a protein that is not substantially homologous to one of the scaffolds, e.g., a protein that is different from the scaffolds and that is derived from the same or a different organism. Within a protease fusion protein the protease polypeptide can correspond to all or a portion of a parent or scaffold protease protein. In one embodiment, a protease fusion protein comprises at least one biologically-active portion of a protease protein. In another embodiment, a protease fusion protein comprises at least two biologically-active portions of a protease protein. In yet another embodiment, a protease fusion protein comprises at least three biologically-active portions of a protease protein. Within the fusion protein, the term “operatively-linked” is intended to indicate that the protease polypeptide and the non-protease polypeptide are fused in-frame with one another. The non-protease polypeptide can be fused to the N-terminus or C-terminus of the protease polypeptide.

In one embodiment, the fusion protein is a GST-protease fusion protein in which the protease sequences are fused to the N-terminus of the GST (glutathione S-transferase) sequences. Such fusion proteins can facilitate the purification of recombinant protease polypeptides.

In another embodiment, the fusion protein is a Fc fusion in which the protease sequences are fused to the N-terminus of the Fc domain from immunoglobulin G. Such fusion proteins can have better pharmacodynamic properties in vivo.

In another embodiment, the fusion protein is a protease protein containing a heterologous signal sequence at its N-terminus. In certain host cells (e.g., mammalian host cells), expression and/or secretion of protease can be increased through use of a heterologous signal sequence.

A protease chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A protease-encoding nucleic acid can be cloned into such an expression vector such that the fusion moiety is linked in-frame to the protease protein.

Protease Agonists and Antagonists

The invention also pertains to variants of the protease proteins that function as either protease agonists (i.e., mimetics) or as protease antagonists. Variants of the protease protein can be generated by mutagenesis (e.g., discrete point mutation or truncation of the protease protein). An agonist of the protease protein can retain substantially the same, or a subset of, the biological activities of the naturally occurring form of the protease protein. An antagonist of the protease protein can inhibit one or more of the activities of the naturally occurring form of the protease protein by, for example, cleaving the same target protein as the protease protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the protease proteins.

Protease Therapy in Combination with Anti-Cancer Agents

Signaling by vascular endothelial growth factor (VEGF) and its receptors is implicated in pathological angiogenesis and the rapid development of tumor vasculature in cancer. Drugs that block this signaling pathway prevent the growth and maintenance of tumor blood supply, and lead to the systematic death of the tumor. The recent success of the anti-VEGF antibody AVASTIN™ in patients with metastatic colon cancer has validated VEGF as a target for anti-angiogenic therapy of cancer. Despite these encouraging results, tumor progression has still occurred despite anti-VEGF treatment. The mechanisms of antibody affecting VEGF function and how the antibody impedes tumor growth are unknown. Knock down experiments show that blocking VEGF function blocks angiogenesis. Thus the inhibition of angiogenic signaling through VEGFR-2 represents an underdeveloped therapeutic area ideal for the development of engineered proteases with novel targeting.

Due to their catalytic nature and smaller size, engineered proteases promise a new therapeutic treatment with advantages over competing targeted binding proteins. The expected advantages include, but are not limited to: better tumor penetration, better target saturation, higher effectiveness, and potentially lower dosing. Notably, because they bind, hydrolyze, and release, a single protease could cleave and inactivate hundreds to thousands of substrate VEGF receptors, offering substantial therapeutic amplification.

In one embodiment, treatment of a pathology, such as a cancer, is provided comprising administering to a subject in need thereof therapeutically effective amounts of a protease that specifically cleaves and inactivates the signaling of the VEGF/VEGFR-2 complex, such as a in combination with at least one anti-cancer agent. Antiangiogenic therapy has proven successful against both solid cancers and hematological malignancies. See, e.g., Ribatti et al. 2003 J Hematother Stem Cell Res. 12(1), 11-22. Therefore, compositions of the invention provided as antiangiogenic therapy will facilitate the treatment of both hematological and sold tissue malignancies. Compositions and methods of treatment provided in the invention may be administered alone or in combination with any other appropriate anti-cancer treatment known to one skilled in the art. For example, the wild-type protease and muteins of the invention can be administered in combination with or in place of AVASTIN™ in any therapy where AVASTIN™ administration provides therapeutic benefit.

In one embodiment, the anti-cancer agent is at least one chemotherapeutic agent. In a related embodiment, the administering of the protease is in combination with at least one radiotherapy. Administration of the combination therapy will attenuate the angiogenic signal and create a pool of soluble receptor that lowers free VEGF levels. In a specific embodiment, a variant granzyme B protease of the invention has an in vitro specificity that matches a critical region of the receptor, the Flk-1/KDR stalk, over a six amino acid region.

The serine protease-like mutein polypeptides of the invention may be administered in a composition containing more than one therapeutic agent. The therapeutic agents may be the same or different, and may be, for example, therapeutic radionuclides, drugs, hormones, hormone antagonists, receptor antagonists, enzymes or proenzymes activated by another agent, autocrines, cytokines or any suitable anti-cancer agent known to those skilled in the art. In one embodiment, the anti-cancer agent co-administered with the wild-type or mutein protease is AVASTIN™. Toxins also can be used in the methods of the present invention. Other therapeutic agents useful in the present invention include anti-DNA, anti-RNA, radiolabeled oligonucleotides, such as antisense oligonucleotides, anti-protein and anti-chromatin cytotoxic or antimicrobial agents. Other therapeutic agents are known to those skilled in the art, and the use of such other therapeutic agents in accordance with the present invention is specifically contemplated.

The antitumor agent may be one of numerous chemotherapy agents such as an alkylating agent, an antimetabolite, a hormonal agent, an antibiotic, an antibody, an anti-cancer biological, Gleevec, colchicine, a vinca alkaloid, L-asparaginase, procarbazine, hydroxyurea, mitotane, nitrosoureas or an imidazole carboxamide. Suitable agents are those agents that promote depolarization of tubulin or prohibit tumor cell proliferation. Chemotherapeutic agents contemplated as within the scope of the invention include, but are not limited to, anti-cancer agents listed in the Orange Book of Approved Drug Products With Therapeutic Equivalence Evaluations, as compiled by the Food and Drug Administration and the U.S. Department of Health and Human Services. In addition to the above chemotherapy agents, the serine protease-like proteases of the invention may also be administered together with radiation therapy treatment. Additional treatments known in the art are contemplated as being within the scope of the invention.

The therapeutic agent may be a chemotherapeutic agent. Chemotherapeutic agents are known in the art and include at least the taxanes, nitrogen mustards, ethylenimine derivatives, alkyl sulfonates, nitrosoureas, triazenes; folic acid analogs, pyrimidine analogs, purine analogs, vinca alkaloids, antibiotics, enzymes, platinum coordination complexes, substituted urea, methyl hydrazine derivatives, adrenocortical suppressants, or antagonists. More specifically, the chemotherapeutic agents may be one or more agents chosen from the non-limiting group of steroids, progestins, estrogens, antiestrogens, or androgens. Even more specifically, the chemotherapy agents may be azaribine, bleomycin, bryostatin-1, busulfan, carmustine, chlorambucil, cisplatin, CPT-11, cyclophosphamide, cytarabine, dacarbazine, dactinomycin, daunorubicin, dexamethasone, diethylstilbestrol, doxorubicin, ethinyl estradiol, etoposide, fluorouracil, fluoxymesterone, gemcitabine, hydroxyprogesterone caproate, hydroxyurea, L-asparaginase, leucovorin, lomustine, mechlorethamine, medroprogesterone acetate, megestrol acetate, melphalan, mercaptopurine, methotrexate, methotrexate, mithramycin, mitomycin, mitotane, phenyl butyrate, prednisone, procarbazine, semustine streptozocin, tamoxifen, taxanes, taxol, testosterone propionate, thalidomide, thioguanine, thiotepa, uracil mustard, vinblastine, or vincristine. The use of any combinations of chemotherapy agents is also contemplated. The administration of the chemotherapeutic agent may be before, during or after the administration of the serine protease-like mutein polypeptide.

Other suitable therapeutic agents for use in combination or for co-administration with the proteases of the invention are selected from the group consisting of radioisotope, boron addend, immunomodulator, toxin, photoactive agent or dye, cancer chemotherapeutic drug, antiviral drug, antifungal drug, antibacterial drug, antiprotozoal drug and chemosensitizing agent (See, U.S. Pat. Nos. 4,925,648 and 4,932,412). Suitable chemotherapeutic agents are described, for example, in REMINGTON'S PHARMACEUTICAL SCIENCES, 19th Ed. (Mack Publishing Co. 1995), and in Goodman and Gilman's THE PHARMACOLOGICAL BASIS OF THERAPEUTICS (Goodman et al., Eds. Macmillan Publishing Co., New York, 1980 and 2001 editions). Other suitable chemotherapeutic agents, such as experimental drugs, are known to those of skill in the art. Moreover a suitable therapeutic radioisotope is selected from the group consisting of ax-emitters, β-emitters, γ-emitters, Auger electron emitters, neutron capturing agents that emit α-particles and radioisotopes that decay by electron capture. Preferably, the radioisotope is selected from the group consisting of ²²⁵Ac, ¹⁹⁸Au, ³²P, ¹³¹I, ¹³¹I, ⁹⁰Y, ¹⁸⁶Re, ¹⁸⁸Re, ⁶⁷Cu, ¹⁷⁷Lu, ²¹³Bi, ¹⁰B, and ²¹¹At.

Where more than one therapeutic agent is used in combination with the proteases of the invention, they may be of the same class or type or may be from different classes or types. For example, the therapeutic agents may comprise different radionuclides, or a drug and a radionuclide.

In another embodiment, different isotopes that are effective over different distances as a result of their individual energy emissions are used as first and second therapeutic agents in combination with the proteases of the invention. Such agents can be used to achieve more effective treatment of tumors, and are useful in patients presenting with multiple tumors of differing sizes, as in normal clinical circumstances.

Few of the available isotopes are useful for treating the very smallest tumor deposits and single cells. In these situations, a drug or toxin may be a more useful therapeutic agent for co-administration with a protease of the invention. Accordingly, in some embodiments of the present invention, isotopes are used in combination with non-isotopic species such as drugs, toxins, and neutron capture agents and co-administered with a protease of the invention. Many drugs and toxins are known which have cytotoxic effects on cells, and can be used in combination with the proteases of the present invention. They are to be found in compendia of drugs and toxins, such as the Merck Index, Goodman and Gilman, and the like, and in the references cited above.

Drugs that interfere with intracellular protein synthesis can also be used in combination with a protease in the therapeutic the methods of the present invention; such drugs are known to those skilled in the art and include puromycin, cycloheximide, and ribonuclease.

The therapeutic methods of the invention may be used for cancer therapy. It is well known that radioisotopes, drugs, and toxins can be conjugated to antibodies or antibody fragments which specifically bind to markers which are produced by or associated with cancer cells, and that such antibody conjugates can be used to target the radioisotopes, drugs or toxins to tumor sites to enhance their therapeutic efficacy and minimize side effects. Examples of these agents and methods are reviewed in Wawrzynczak and Thorpe (in Introduction to the Cellular and Molecular Biology of Cancer, L. M. Franks and N. M. Teich, eds, Chapter 18, pp. 378-410, Oxford University Press. Oxford, 1986), in Immunoconjugates: Antibody Conjugates in Radioimaging and Therapy of Cancer (C. W. Vogel, ed., 3-300, Oxford University Press, N.Y., 1987), in Dillman, R. O. (CRC Critical Reviews in Oncology/Hematology 1:357, CRC Press, Inc., 1984), in Pastan et al. (Cell 47:641, 1986) in Vitetta et al. (Science 238:1098-1104, 1987) and in Brady et al. (Int. J. Rad. Oncol. Biol. Phys. 13:1535-1544, 1987). Other examples of the use of immunoconjugates for cancer and other forms of therapy have been disclosed, inter alia, in U.S. Pat. Nos. 4,331,647, 4,348,376, 4,361,544, 4,468,457, 4,444,744, 4,460,459, 4,460,561 4,624,846, 4,818,709, 4,046,722, 4,671,958, 4,046,784, 5,332,567, 5,443,953, 5,541,297, 5,601,825, 5,635,603, 5,637,288, 5,677,427, 5,686,578, 5,698,178, 5,789,554, 5,922,302, 6,187,287, and 6,319,500.

Additionally, the treatment methods of the invention include those in which a protease of the invention is used in combination with other compounds or techniques for preventing, mitigating or reversing the side effects of certain cytotoxic agents. Examples of such combinations include, e.g., administration of IL-1 together with an antibody for rapid clearance, as described in e.g., U.S. Pat. No. 4,624,846. Such administration can be performed from 3 to 72 hours after administration of a primary therapeutic treatment with a granzyme B mutein or MT-SP1 mutein in combination with a anti-cancer agent (e.g., with a radioisotope, drug or toxin as the cytotoxic component). This can be used to enhance clearance of the conjugate, drug or toxin from the circulation and to mitigate or reverse myeloid and other hematopoietic toxicity caused by the therapeutic agent.

In another aspect of the invention, and as noted above, cancer therapy may involve a combination of more than one tumoricidal agent, e.g., a drug and a radioisotope, or a radioisotope and a Boron-10 agent for neutron-activated therapy, or a drug and a biological response modifier, or a fusion molecule conjugate and a biological response modifier. The cytokine can be integrated into such a therapeutic regimen to maximize the efficacy of each component thereof.

Similarly, certain antileukemic and antilymphoma antibodies conjugated with radioisotopes that are β or α emitters may induce myeloid and other hematopoietic side effects when these agents are not solely directed to the tumor cells. This is observed particularly when the tumor cells are in the circulation and in the blood-forming organs. Concomitant and/or subsequent administration of at least one hematopoietic cytokine (e.g., growth factors, such as colony stimulating factors, such as G-CSF and GM-CSF) is preferred to reduce or ameliorate the hematopoietic side effects, while augmenting the anticancer effects.

It is well known in the art that various methods of radionuclide therapy can be used for the treatment of cancer and other pathological conditions, as described, e.g., in Harbert, “Nuclear Medicine Therapy”, New York, Thieme Medical Publishers, 1087, pp. 1-340. A clinician experienced in these procedures will readily be able to adapt the cytokine adjuvant therapy described herein to such procedures to mitigate any hematopoietic side effects thereof. Similarly, therapy with cytotoxic drugs, co-administered with a protease mutein, can be used, e.g., for treatment of cancer, infectious or autoimmune diseases, and for organ rejection therapy. Such treatment is governed by analogous principles to radioisotope therapy with isotopes or radiolabeled antibodies. Thus, the ordinary skilled clinician will be able to adapt the description of cytokine use to mitigate marrow suppression and other such hematopoietic side effects by administration of the cytokine before, during and/or after the primary anti-cancer therapy.

Pharmaceutical Compositions

Sequential or substantially simultaneous administration of each therapeutic protease and other therapeutic agents combined with the protease can be effected by any appropriate route including, but not limited to, oral routes, intravenous routes, intramuscular routes, and direct absorption through mucous membrane tissues. Mutein or wild-type proteases and other therapeutic agents can be administered by the same route or by different routes. For example, Mutein or wild-type proteases may be administered by intravenous injection while the other therapeutic agent(s) of the combination may be administered orally. Alternatively, for example, the other therapeutic agent(s) may be administered by intravenous injection. The sequence in which the therapeutic agents are administered is not narrowly critical.

Administration of mutein or wild-type proteases also can be accompanied by the administration of the other therapeutic agents as described above in further combination with other biologically active ingredients and non-drug therapies (e.g., surgery or radiation treatment.) or with non-drug therapies alone with mutein or wild-type proteases. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Thus, mutein or wild-type proteases and the other pharmacologically active agent may be administered to a patient simultaneously, sequentially or in combination. If administered sequentially, the time between administrations generally varies from 0.1 to about 48 hours. It will be appreciated that when using mutein or wild-type proteases with other therapeutic agent(s), they may be in the same pharmaceutically acceptable carrier and therefore administered simultaneously. They may be in separate pharmaceutical carriers such as conventional oral dosage forms which are taken simultaneously.

A therapy for a angiogenic condition includes mutein or wild-type proteases and AVASTIN™. In one embodiment, this condition is cancer.

A therapy for cancer, inflammation, diabetes or macular degeneration includes mutein or wild-type proteases. In another embodiment, this therapy further includes another therapeutic as defined above.

Advantages attributed to the administration of mutein or wild-type proteases and at least a second agent as part of a specific treatment regimen includes, but is not limited to, pharmacokinetic or pharmacodynamic co-action resulting from the combination of therapeutic agents. In one embodiment, the co-action of the therapeutic agents is additive. In another embodiment, the co-action of the therapeutic agents is synergistic. In another embodiment, the co-action of the therapeutic agents improves the therapeutic regimen of one or both of the agents.

The invention further relates to kits for treating patients having an angiogenic condition, such as cancer, comprising a therapeutically effective dose of mutein or wild-type proteases for treating or at least partially alleviating the symptoms of the condition (e.g., AVASTIN™), either in the same or separate packaging, and instructions for its use.

The present invention is suitable for the reduction of cancer symptoms. These cancer symptoms include blood in the urine, pain or burning upon urination, frequent urination, cloudy urine, pain in the bone or swelling around the affected site, fractures in bones, weakness, fatigue, weight loss, repeated infections, nausea, vomiting, constipation, problems with urination, weakness or numbness in the legs, bumps and bruises that persist, dizziness, drowsiness, abnormal eye movements or changes in vision, weakness, loss of feeling in arms or legs or difficulties in walking, fits or convulsions, changes in personality, memory or speech, headaches that tend to be worse in the morning and ease during the day, that may be accompanied by nausea or vomiting, a lump or thickening of the breast, discharge from the nipple, change in the skin of the breast, a feeling of heat, or enlarged lymph nodes under the arm, rectal bleeding (red blood in stools or black stools), abdominal cramps, constipation alternating with diarrhea, weight loss, loss of appetite, weakness, pallid complexion, dull ache or pain in the back or side, lump in kidney area, sometimes accompanied by high blood pressure or abnormality in red blood cell count, weakness, paleness, fever and flu-like symptoms, bruising and prolonged bleeding, enlarged lymph nodes, spleen, liver, pain in bones and joints, frequent infections, weight loss, night sweats, wheezing, persistent cough for months, blood-streaked sputum, persistent ache in chest, congestion in lungs, enlarged lymph nodes in the neck, change in mole or other bump on the skin, including bleeding or change in size, shape, color, or texture, painless swelling in the lymph nodes in the neck, underarm, or groin, persistent fever, feeling of fatigue, unexplained weight loss, itchy skin and rashes, small lumps in skin, bone pain, swelling in the abdomen, liver or spleen enlargement, a lump in the mouth, ulceration of the lip, tongue or inside of the mouth that does not heal within a couple of weeks, dentures that no longer fit well, oral pain, bleeding, foul breath, loose teeth, changes in speech, abdominal swelling, abnormal vaginal bleeding, digestive discomfort, upper abdominal pain, unexplained weight loss, pain near the center of the back, intolerance of fatty foods, yellowing of the skin, abdominal masses, enlargement of liver and spleen, urination difficulties due to blockage of the urethra, bladder retains urine, creating frequent feelings of urgency to urinate, especially at night, bladder not emptying completely, burning or painful urination, bloody urine, tenderness over the bladder, dull ache in the pelvis or back, indigestion or heartburn, discomfort or pain in the abdomen, nausea and vomiting, diarrhea or constipation, bloating after meals, loss of appetite, weakness and fatigue, bleeding—vomiting blood or blood in the stool, abnormal vaginal bleeding, a watery bloody discharge in postmenopausal women, a painful urination, pain during intercourse, and pain in pelvic area

The present invention is suitable for the reduction of macular degeneration symptoms. These macular degeneration symptoms include blurring of vision, lines forming in vision and gradual or quick loss of vision.

The present invention is suitable for the reduction of diabetes symptoms. These diabetes symptoms include loss of vision and blindness.

Preferably, treatment should continue as long as symptoms are suspected or observed To evaluate whether a patient is benefiting from the (treatment), one would examine the patient's symptoms in a quantitative way, by decrease in the frequency of relapses, or increase in the time to sustained progression, or improvement and compare the patient's status measurement before and after treatment. In a successful treatment, the patient status will have improved. Measurement number or frequency of relapses will have decreased, or the time to sustained progression will have increased.

As for every drug, the dosage is an important part of the success of the treatment and the health of the patient. In every case, in the specified range, the physician has to determine the best dosage for a given patient, according to gender, age, weight, height, pathological state and other parameters.

The pharmaceutical compositions of the present invention contain a therapeutically effective amount of mutein or wild-type proteases. The amount of the compound will depend on the patient being treated. The patient's weight, severity of illness, manner of administration and judgment of the prescribing physician should be taken into account in deciding the proper amount. The determination of a therapeutically effective amount of mutein or wild-type proteases or other therapeutic agent is well within the capabilities of one with skill in the art.

In some cases, it may be necessary to use dosages outside of the ranges stated in pharmaceutical packaging insert to treat a patient. Those cases will be apparent to the prescribing physician. Where it is necessary, a physician will also know how and when to interrupt, adjust or terminate treatment in conjunction with a response of a particular patient.

Formulation (Separately or Together) and Administration

The compounds of the present invention are administered separately or co-formulated in a suitable co-formulated dosage form. Compounds, including those used in combination therapies are administered to a patient in the form of a pharmaceutically acceptable salt or in a pharmaceutical composition. A compound that is administered in a pharmaceutical composition is mixed with a suitable carrier or excipient such that a therapeutically effective amount is present in the composition. The term “therapeutically effective amount” refers to an amount of the compound that is necessary to achieve a desired endpoint (e.g., decreasing symptoms associated with cancer).

A variety of preparations can be used to formulate pharmaceutical compositions containing mutein or wild-type proteases and other therapeutic agents. Techniques for formulation and administration may be found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants and aerosols are examples of such formulations. The formulations can be administered in either a local or systemic manner or in a depot or sustained release fashion. Administration of the composition can be performed in a variety of ways. The compositions and combination therapies of the invention may be administered in combination with a variety of pharmaceutical excipients, including stabilizing agents, carriers and/or encapsulation formulations as described herein.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including creams, lotions, mouthwashes, inhalants and the like.

For human administration, preparations should meet sterility, pyrogenicity, general safety and purity standards as required by the FDA.

Administration of compounds alone or in combination therapies may be, e.g., subcutaneous, intramuscular or intravenous injection, or any other suitable route of administration. A particularly convenient frequency for the administration of the compounds of the invention is once a day.

Upon formulation, therapeutics will be administered in a manner compatible with the dosage formulation, and in such amount as is pharmacologically effective. The formulations are easily administered in a variety of dosage forms, such as the injectable solutions described, but drug release capsules and the like can also be employed. In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

A carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Suitable preservatives for use in solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

The compounds and combination therapies of the invention can be formulated by dissolving, suspending or emulsifying in an aqueous or nonaqueous solvent. Vegetable (e.g., sesame oil, peanut oil) or similar oils, synthetic aliphatic acid glycerides, esters of higher aliphatic acids and propylene glycol are examples of nonaqueous solvents. Aqueous solutions such as Hank's solution, Ringer's solution or physiological saline buffer can also be used. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for subcutaneous or intramuscular injection is also contemplated. In this regard, the use of DMSO as solvent is preferred as this will result in extremely rapid penetration, delivering high concentrations of the active compound(s) or agent(s) to a small area.

Where one or both active ingredients of the combination therapy is given orally, it can be formulated through combination with pharmaceutically acceptable carriers that are well known in the art. The carriers enable the compound to be formulated, for example, as a tablet, pill, capsule, solution, suspension, sustained release formulation; powder, liquid or gel for oral ingestion by the patient. Oral use formulations can be obtained in a variety of ways, including mixing the compound with a solid excipient, optionally grinding the resulting mixture, adding suitable auxiliaries and processing the granule mixture. The following list includes examples of excipients that can be used in an oral formulation: sugars such as lactose, sucrose, mannitol or sorbitol; cellulose preparations such as maize starch, wheat starch, potato starch, gelatin, gum tragacanth, methyl cellulose, hydroxypropylmethylcellulose, sodium carboxymethylcellulose and polyvinylpyrrolidone (PVP). Oral formulations include such normally employed excipients as, for example, pharmaceutical grades of mannitol, lactose, starch, magnesium stearate, sodium saccharine, cellulose, magnesium carbonate and the like.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabensas preservatives, a dye and flavoring, such as cherry or orange flavor.

Additional formulations suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The subject treated by the methods of the invention is a mammal, more preferably a human. The following properties or applications of these methods will essentially be described for humans although they may also be applied to non-human mammals, e.g., apes, monkeys, dogs, mice, etc. The invention therefore can also be used in a veterinarian context.

The following examples are nonlimiting and meant only to illustrate various aspects of the invention.

### EXAMPLES

### Example 1

### Methods of Cloning and Characterizing Engineered Granzyme B Protease with Altered Substrate Specificity Based on Well Understood Starting Scaffolds

The serine protease granzyme B has been chosen as the scaffold protease for mutagenesis towards specific proteolysis of the VEGF and VEGFR in part because it has been well characterized with biochemical and structural techniques [Harris et al., J Biol Chem, Vol. 273, Issue 42, 27364-27373, Oct. 16, 1998; Waugh, S. M., Harris, J. L., Fletterick, R., and Craik, C. S. (2000) Nature Structural Biology 7(9), 762-765.].

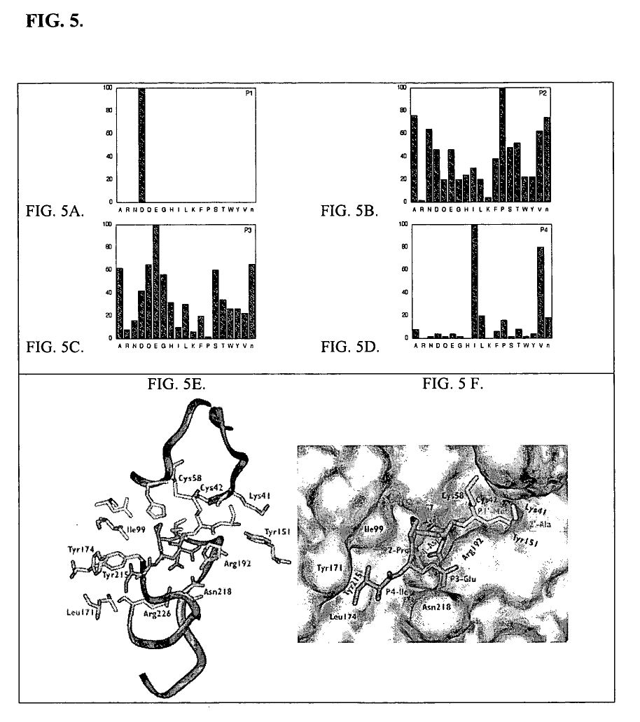

Granzyme B is a serine protease involved in cytotoxic lymphocyte induced cell death of tumor or virally infected cells. It induces the apoptotic cascade through caspase activation and specific substrate hydrolysis at aspartate residues. Its optimal substrate is Ile-Glu-Pro-Asp/Trp-Gly (SEQ ID NO:25), as determined using PSSCL [Thornberry, N. et al. (1997) Journal of Biological Chemistry 272(29), 17907-11.] (FIGS. 5A-5D) and substrate-phage display (Harris 1998). Numerous amino acids with contributions to the narrow extended specificity of granzyme B were identified through the three-dimensional structures of granzyme B [Estebanez-Perpina, E., et al. (2000) Biol Chem 381(12), 1203-14.; Rotonda, J., et al. (2001) Chem Bio18(4), 357-68.] and its complex with ecotin [81-84 IEDP][Waugh, 2000].

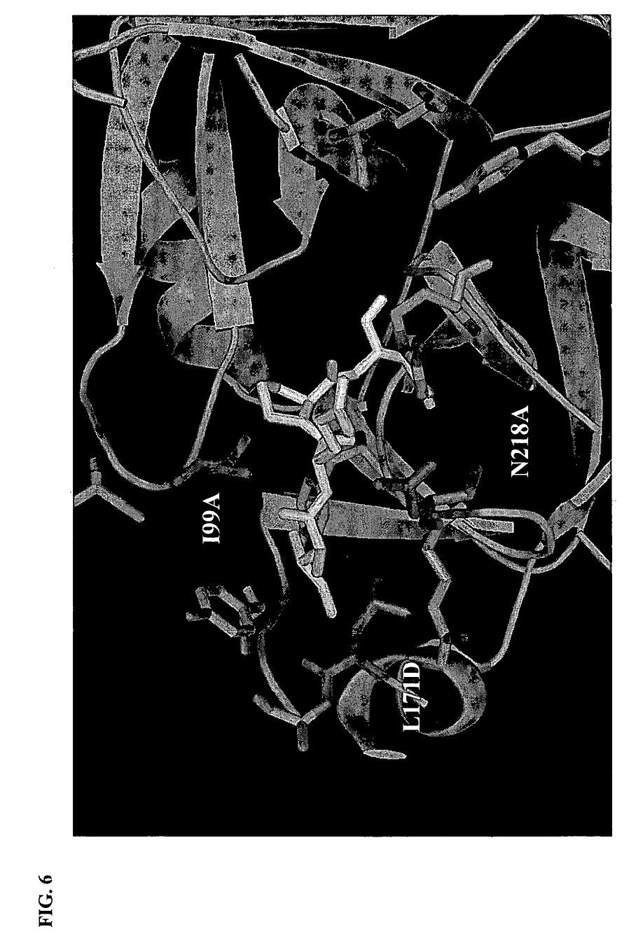

PSSCL results for rat granzyme B. The activity in each subsite well is normalized to 100%. The three dimensional structure of rat granzyme B shown as a ribbon and as a surface (FIGS. 5E-5F). The N-terminal side of the ecotin inhibitor's substrate-like binding loop is shown in medium grey, and the C-terminal side of the substrate is modeled in light grey. The ribbon structure of granzyme B mutein CB06 is provided in FIG. 6.

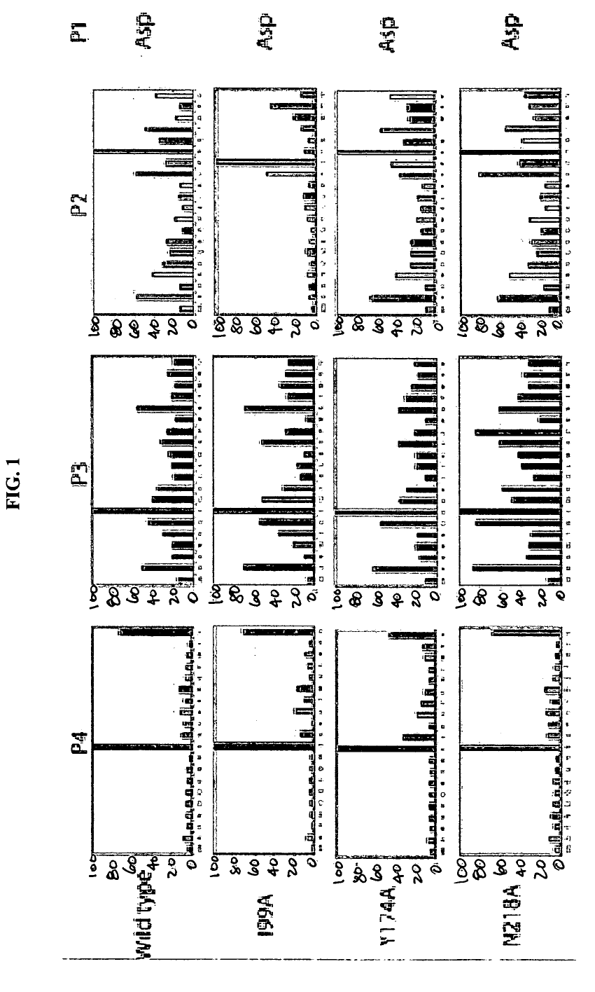

Variants of the above proteases have been created and characterized. Each protease has been expressed and purified, as provided herein. Initial activity to verify activity and specificity of each batch have been performed, and sample results are provided in FIGS. 1-14.

### Example 2

### Expression and Purification of Granzyme B

A mutated granzyme B polypeptide (“mutein”) may contain a single mutation per polypeptide, or may contain two or more mutated residues in any combination provided in Tables 12, 13 and 14.

Wild-type and mutant granzyme B are cloned into the pPICZalphaA yeast expression vector (Invitrogen) flush against the alpha factor secretion signal, and transformed into X33 cells. Cells are grown in 250 mL cultures to an OD of 0.6 and expression of the secreted, active protease is induced by adding methanol to a final concentration of 0.5%. After 48 to 72 hours, the yeast are pelleted by centrifugation and the supernatant is loaded onto a SP-Sepharose Fast Flow resin (Amersham). The column is washed with three volumes each of 50 mM MES pH 6.0, 50 mM NaCl and 200 mM NaCal. The protease is then eluted with three (3) column volumes of 50 mM MES pH6.0, 1 M NaCl, and the column is washed with 50 mM MES pH 6.0, 2M NaCl. The protease is then concentrated to a volume of approximately 1 mL and exchanged into a storage buffer consisting of 50 mM MES pH 6.0, 100 mM NaCl.

Result. Multimilligram quantities are obtained using a yeast expression system. The protease is secreted in its mature, catalytically active form and purified with a one-column purification procedure.

### Example 3

### Synthesis and Screening of Combinatorial Libraries for Characterization of Protease Wild-Type and Muteins

Fixed P1 Amino Acid Method

Individual P1-substituted Fmoc-amino acid ACC-resin (ca. 25 mg, 0.013 mmol) was added to wells of a Multi-Chem 96-well reaction apparatus. The resin-containing wells were solvated with DMF (0.5 mL). A 20% piperidine in DMF solution (0.5 mL) was added followed by agitation for 30 min. The wells of the reaction block were filtered and washed with DMF (3×0.5 mL). In order to introduce the randomized P2 position, an isokinetic mixture (Ostresh, J. M., et al., (1994) Biopolymers 34:1681-9) of Fmoc-amino acids (4.8 mmol, 10 equiv/well; Fmoc-amino acid, mol %: Fmoc-Ala-OH, 3.4; Fmoc-Arg(Pbf)-OH, 6.5; Fmoc-Asn(Trt)-OH, 5.3; Fmoc-Asp(O-t-Bu)-OH, 3.5; Fmoc-Glu(O-t-Bu)-OH, 3.6; Fmoc-Gln(Trt)-OH, 5.3; Fmoc-Gly-OH, 2.9; Fmoc-His(Trt)-OH, 3.5; Fmoc-Ile-OH, 17.4; Fmoc-Leu-OH, 4.9; Fmoc-Lys(Boc)-OH, 6.2; Fmoc-Nle-OH, 3.8; Fmoc-Phe-OH, 2.5; Fmoc-Pro-OH, 4.3; Fmoc-Ser(O-t-Bu)-OH, 2.8; Fmoc-Thr(O-t-Bu)-OH, 4.8; Fmoc-Trp(Boc)-OH, 3.8; Fmoc-Tyr(O-t-Bu)-OH, 4.1; Fmoc-Val-OH, 11.3) was pre-activated with DICI (390 μL, 2.5 mmol), and HOBt (340 mg, 2.5 mmol) in DMF (10 mL). The solution (0.5 mL) was added to each of the wells. The reaction block was agitated for 3 h, filtered, and washed with DMF (3×0.5 mL). The randomized P3 and P4 positions were incorporated in the same manner. The Fmoc of the P4 amino acid was removed and the resin was washed with DMF (3×0.5 mL), and treated with 0.5 mL of a capping solution of AcOH (150 μL, 2.5 mmol), HOBt (340 mg, 2.5 mmol) and DICI (390 μL, 2.5 mmol) in DMF (10 mL). After 4 h of agitation, the resin was washed with DMF (3×0.5 mL), CH₂Cl₂ (3×0.5 mL), and treated with a solution of 95:2.5:2.5 TFA/TIS/H₂O. After incubating for 1 h the reaction block was opened and placed on a 96 deep-well titer plate and the wells were washed with additional cleavage solution (2×0.5 mL). The collection plate was concentrated, and the substrate-containing wells were diluted with EtOH (0.5 mL) and concentrated twice. The contents of the individual wells were lyophilized from CH₃CN:H₂O mixtures. The total amount of substrate in each well was conservatively estimated to be 0.0063 mmol (50%) based upon yields of single substrates.

P1-Diverse Amino Acid Method 7-Fmoc-aminocoumarin-4-acetic acid was prepared by treating 7-aminocoumarin-4-acetic acid with Fmoc-Cl. 7-Aminocoumarin-4-acetic acid (10.0 g, 45.6 mmol) and H₂O (228 ml) were mixed. NaHCO₃ (3.92 g, 45.6 mmol) was added in small portions followed by the addition of acetone (228 ml). The solution was cooled with an ice bath, and Fmoc-Cl (10.7 g, 41.5 mmol) was added with stirring over the course of 1 h. The ice bath was removed and the solution was stirred overnight. The acetone was removed with rotary evaporation and the resulting gummy solid was collected by filtration and washed with several portions of hexane. ACC-resin was prepared by condensation of Rink Amide AM resin with 7-Fmoc-aminocoumarin-4-acetic acid. Rink Amide AM resin (21 g, 17 mmol) was solvated with DMF (200 ml). The mixture was agitated for 30 min and filtered with a filter cannula, whereupon 20% piperidine in DMF (200 ml) was added. After agitation for 25 min, the resin was filtered and washed with DMF (3 times, 200 ml each). 7-Fmoc-aminocoumarin-4-acetic acid (15 g, 34 mmol), HOBt (4.6 g, 34 mmol), and DMF (150 ml) were added, followed by diisopropylcarbodiimide (DICI) (5.3 ml, 34 mmol). The mixture was agitated overnight, filtered, washed (DMF, three times with 200 ml; tetrahydrofuran, three times with 200 ml; MeOH, three times with 200 ml), and dried over P₂O₅. The substitution level of the resin was 0.58 mmol/g (>95%) as determined by Fmoc analysis.

P1-Diverse Library Synthesis

Individual P1-substituted Fmoc-amino acid ACC-resin (˜25 mg, 0.013 mmol) was added to wells of a MultiChem 96-well reaction apparatus. The resin-containing wells were solvated with DMF (0.5 ml). After filtration, 20% piperidine in DMF solution (0.5 ml) was added, followed by agitation for 30 min. The wells of the reaction block were filtered and washed with DMF (three times with 0.5 ml). To introduce the randomized P2 position, an isokinetic mixture of Fmoc-amino acids [4.8 mmol, 10 eq per well; Fmoc-amino acid, mol %: Fmoc-Ala-OH, 3.4; Fmoc-Arg(Pbf)-OH, 6.5; Fmoc-Asn(Trt)-OH, 5.3; Fmoc-Asp(O-t-Bu)-OH, 3.5; Fmoc-Glu(O-t-Bu)-OH, 3.6; Fmoc-Gln(Trt)-OH, 5.3; Fmoc-Gly-OH, 2.9; Fmoc-His(Trt)-OH, 3.5; Fmoc-Ile-OH, 17.4; Fmoc-Leu-OH, 4.9; Fmoc-Lys(Boc)-OH, 6.2; Fmoc-Nle-OH, 3.8; Fmoc-Phe-OH, 2.5; Fmoc-Pro-OH, 4.3; Fmoc-Ser(O-t-Bu)-OH, 2.8; Fmoc-Thr(O-t-Bu)-OH, 4.8; Fmoc-Trp(Boc)-OH, 3.8; Fmoc-Tyr(O-t-Bu)-OH, 4.1; Fmoc-Val-OH, 11.3] was preactivated with DICI (390 μl, 2.5 mmol), and HOBt (340 mg, 2.5 mmol) in DMF (10 ml). The solution (0.5 ml) was added to each of the wells. The reaction block was agitated for 3 h, filtered, and washed with DMF (three times with 0.5 ml). The randomized P3 and P4 positions were incorporated in the same manner. The Fmoc of the P4 amino acid was removed and the resin was washed with DMF (three times with 0.5 ml) and treated with 0.5 ml of a capping solution of AcOH (150 μl, 2.5 mmol), HOBt (340 mg, 2.5 mmol), and DICI (390 μl, 2.5 mmol) in DMF (10 ml). After 4 h of agitation, the resin was washed with DMF (three times with 0.5 ml) and CH₂Cl₂ (three times with 0.5 ml), and treated with a solution of 95:2.5:2.5 TFA/TIS/H₂O. After incubation for 1 h the reaction block was opened and placed on a 96-deep-well titer plate and the wells were washed with additional cleavage solution (twice with 0.5 ml). The collection plate was concentrated, and the material in the substrate-containing wells was diluted with EtOH (0.5 ml) and concentrated twice. The contents of the individual wells were lyophilized from CH₃CN/H₂O mixtures. The total amount of substrate in each well was conservatively estimated to be 0.0063 mmol (50%) on the basis of yields of single substrates.

Screening Methods Using Both Libraries

Multigram quantities of P1-substituted ACC-resin can be synthesized by the methods described. Fmoc-amino acid-substituted ACC resin was placed in 57 wells of a 96-well reaction block: sub-libraries were denoted by the second fixed position (P4, P3, P2) of 19 amino acids (cysteine was omitted and norleucine was substituted for methionine). Synthesis, capping, and cleavage of the substrates were identical to those described in the previous section, with the exception that for P2, P3, and P4 sub-libraries, individual amino acids (5 eq of Fmoc-amino acid monomer, 5 eq of DICI, and 5 eq of HOBt in DMF), rather than isokinetic mixtures, were incorporated in the spatially addressed P2, P3, or P4 positions.

Preparation of the complete diverse and P1-fixed combinatorial libraries was carried out as described above. The library was aliquoted into 96-well plates to a final concentration of 250 μM. Variant proteases were diluted in protease activity buffer (50 mM Na Hepes, pH 8.0, 100 mM NaCl, 0.01% Tween-20) to concentrations between 50 nM and 1 μM. Initial activity against Ac-QGR-AMC was used to adjust the variant protease concentration to one approximately equal to 50 nM wild-type protease. Enzymatic activity in the P1-Arg library was assayed for one hour at 30° C. on a Spectra-Max Delta flourimeter (Molecular Devices). Excitation and emission were measured at 380 nm and 460 nm, respectively.

Synthesis and Fluorescence Screening of Libraries.

P1-Diverse Library

A(i). Synthesis

P1-diverse libraries were synthesized as provided above. The specificity of the various MT-SP1 muteins were characterized as compared to wild-type protease.

A (ii). Enzymatic Assay of Library

The concentration of proteolytic enzymes was determined by absorbance measured at 280 nm (Gill, S. C., et al., (1989) Anal Biochem 182:319-26). The proportion of catalytically active thrombin, plasmin, trypsin, uPA, tPA, and chymotrypsin was quantitated by active-site titration with MUGB or MUTMAC (Jameson, G. W., et al., (1973) Biochemical Journal 131:107-117).

Substrates from the PSSCLs were dissolved in DMSO. Approximately 1.0×10⁻⁹ mol of each P1-Lys, P1-Arg, or P1-Leu sub-library (361 compounds) was added to 57 wells of a 96-well microfluor plate (Dynex Technologies, Chantilly, Va.) for a final concentration of 0.1 μM. Approximately 1.0×10⁻¹⁰ mol of each P1-diverse sub-library (6859 compounds) was added to 20 wells of a 96-well plate for a final concentration of 0.01 μM in each compound. Hydrolysis reactions were initiated by the addition of enzyme (0.02 nM-100 nM) and monitored fluorimetrically with a Perkin Elmer LS50B Luminescence Spectrometer, with excitation at 380 nm and emission at 450 nm or 460 nm. Assays of the serine proteases were performed at 25° C. in a buffer containing 50 mM Tris, pH 8.0, 100 mM NaCl, 0.5 mM CaCl₂, 0.01% Tween-20, and 1% DMSO (from substrates). Assay of the cysteine proteases, papain and cruzain, was performed at 25° C. in a buffer containing 100 mM sodium acetate, pH 5.5, 100 mM NaCl, 5 mM DTT, 1 mM EDTA, 0.01% Brij-35, and 1% DMSO (from substrates).

B. Profiling Proteases with a P1-Diverse Library of 137,180 Substrate Sequences

To test the possibility of attaching all amino acids to the P1-site in the substrate sequence a P1-diverse tetrapeptide library was created. The P1-diverse library consists of 20 wells in which only the P1-position is systematically held constant as all amino acids, excluding cysteine and including norleucine. The P2, P3, and P4 positions consist of an equimolar mixture of all amino acids for a total of 6,859 substrate sequences per well. Several serine and cysteine proteases were profiled to test the applicability of this library for the identification of the optimal P1 amino acid. Chymotrypsin showed the expected specificity for large hydrophobic amino acids. Trypsin and thrombin showed preference for P1-basic amino acids (Arg>Lys). Plasmin also showed a preference for basic amino acids (Lys>Arg). Granzyme B, the only known mammalian serine protease to have P1-Asp specificity, showed a distinct preference for aspartic acid over all other amino acids, including the other acidic amino acid, Glu. The P1-profile for human neutrophil elastase has the canonical preference for alanine and valine. The cysteine proteases, papain and cruzain showed the broad P1-substrate sequence specificity that is known for these enzymes, although there is a modest preference for arginine. For example, the MT-SP1 wild-type protease preferred Arg or Lys.

C. Profiling Proteases with the P1-Constant Library

A P1-constant tetrapeptide library is created as disclosed above. The P1-constant library consists of 20 wells in which only the P1-position is systematically held constant as all amino acids, excluding cysteine and including norleucine. The P2, P3, and P4 positions consist of an equimolar mixture of all amino acids for a total of 6,859 substrate sequences per well. Several serine and cysteine proteases were profiled to test the applicability of this library for the identification of the optimal P1 amino acid. For example, MT-SP1 prefers the amino acids Arg and Lys at P1.

### Example 4

### Determination of the Extended Specificity of Granzyme B Variants by PSSCL

The P1-Asp fixed PSSCL library is resuspended in DMSO and arrayed in opaque black 96-well plates at a concentration of 5-10 nanomoles per well. Variant proteases are diluted into 50 mM Tris pH 8, 50 mM NaCl, and 0.01% Tween 20 (granzyme B activation buffer) at a concentration of 10-100 nM. One hundred microliters of the protease solution is added to each well and fluorescence of the ACC leaving group is measured by excitation at 380 nm and emission at 460 nm using a Spectramax fluorescent plate reader (Molecular Devices). The specificity of variant proteases at each of the P4-P2 extended subsites is determined by the rate of increase in the fluorescence over time of each of the arrayed amino acids in the P4-P2 PSSC libraries. The P1 residue is held constant as Asp.

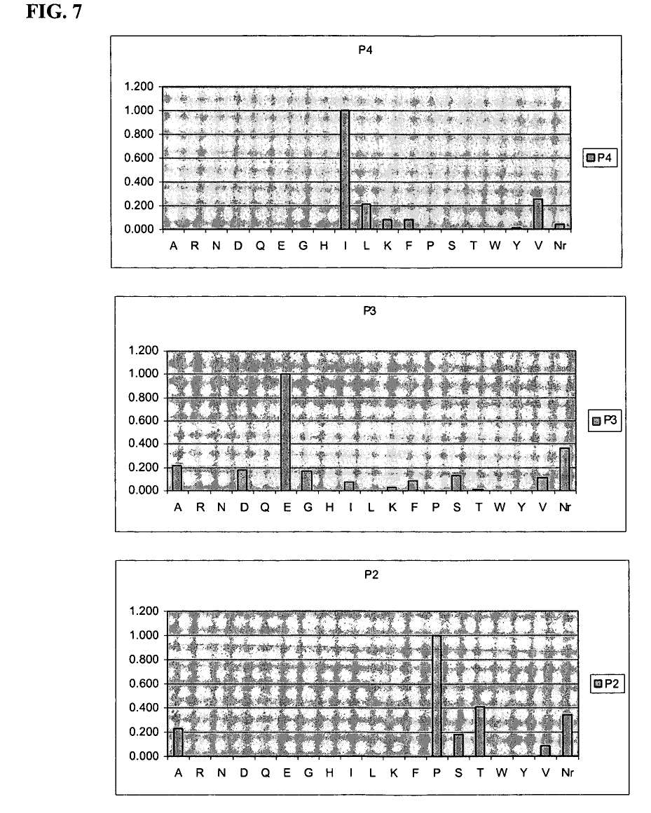

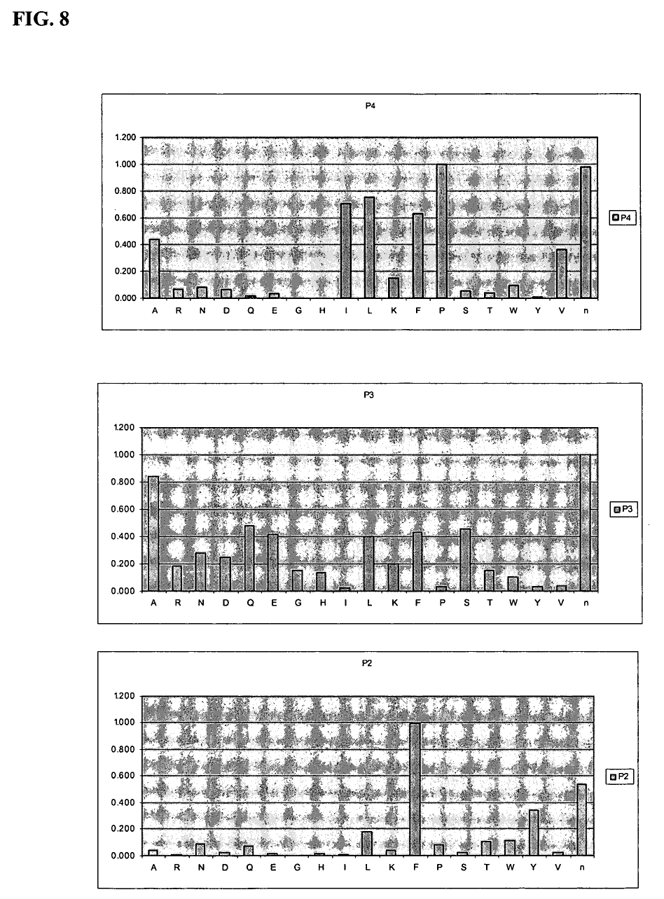

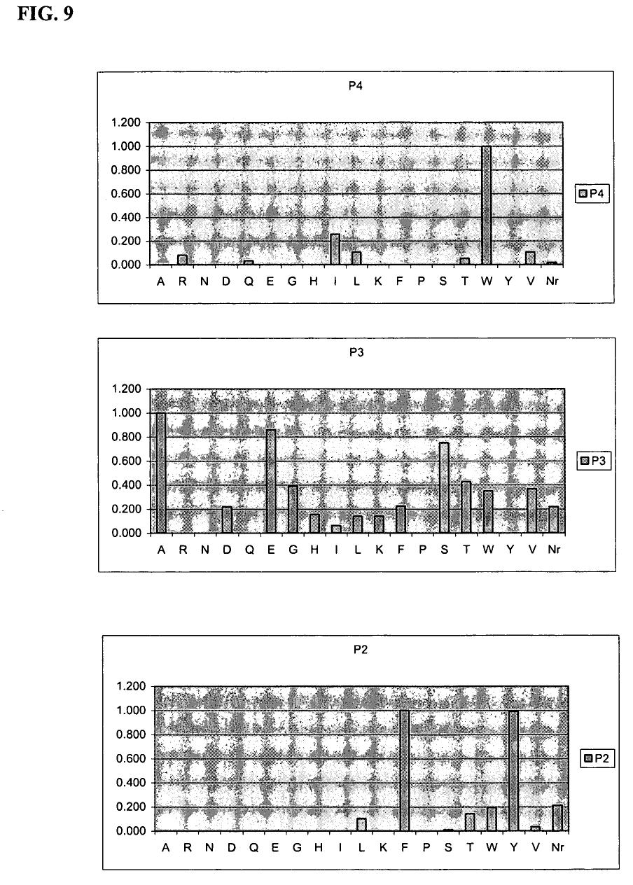

Screening by PSSCL confirms that wild-type granzyme B has a preference for I/V at P4, E at P3, P/T at P2 and D at P1 (FIG. 7), in agreement with published data. See, Thornberry, 1997. A number of muteins were generated (see Tables 12, 13, 14 and 15), to direct them towards potential cleavage sites identified in VEGFR. The CB01 mutein shows a broadened specificity for amino acids at positions P4 and P3, while Phe is now the preferred residue at the P2 position (FIG. 8). The CB06 mutein prefers Trp at P4, has broad specificity at P3, and has equal preference for Phe or Tyr at P2 (FIG. 9).

### Example 5

### VEGFR Cleavage

The polypeptide sequence of VEGF receptor 2 (VEGF-R2/KDR), showing the respective sequences of the extracellular (SEQ ID NO:28) and intracellular (SEQ ID NO:29) domains, is provided in Table 19. Sequences that closely match the P4-P1 native substrate specificity of granzyme B or the novel specificities of CB01 and CB06 are shown in bold, and include VLKD, LVED and WFKD.

TABLE 19
VEGFR2/KDR Substrate Specificity of Targeted GranzymeB Proteases

| | | |
|---|---|---|
| Extracellular | KQSKVLLAVALWLCVETRAASVGLPSVSLDLPRLSIQKDILTIKANTTLQITCRGQRDLD | (SEQ ID NO:28) |
| | WLWPNNQSGSEQRVEVTECSDGLFCKTLTIPKVIGNDTGAYKCFYRETDLASVIYVYVQD | |
| | YRSPFIASVSDQHGVVYITENKNKTVVIPCLGSISNLNVSLCARYPEKRFVPDGNRISWD | |
| | SKKGFTIPSYMISYAGMVFCEAKINDESYQSIMYIVVVVGYRIYDVVLSPSHGIELSVGE | |
| | KLVLNCTARTELNVGIDFNWEYPSSKHQHKKLVNRDLKTQSGSEMKKFLSTLTIDGVTRS | |
| | DQGLYTCAASSGLMTKKNSTFVRVHEKPFVAFGSGMESLVEATVGERVRIPAKYLGYPPP | |
| | EIKWYKNGIPLESNHTIKAGHVLTIMEVSERDTGNYTVILTNPISKEKQSHVVSLVVYVP | |
| | PQIGEKSLISPVDSYQYGTTQTLTCTVYAIPPPHHIHWYWQLEEECANEPSQAVSVTNPY | |
| | PCEEWRSVEDFQGGNKIEVNKNQFALIEGKNKTVSTLVIQAANVSALYKCEAVNKVGRGE | |
| | RVISFHVTRGPEITLQPDMQPTEQESVSLWCTADRSTFENLTWYKLGPQPLPIHVGELPT | |
| | PVCKNLDTLWKLNATMFSNSTNDILIMELKNASLQDQGDYVCLAQDRKTKKRHCVVRQLT | |
| | VLERVAPTITGNLENQTTSIGESIEVSCTASGNPPPQIMWFKDNETLVEDSGIVLKDGNR | |
| | NLTIRRVRKEDEGLYTCQACSVLGCAKVEAFFIIEGAQEKTNLE | |
| Intracellular | IIILVGTAVIAMFFWLLLVIILRTVKRANGGELKTGYLSIVMDPDELFLDEHCERLPYDA | (SEQ ID NO:29) |
| | SKWEFPRDRLKLGKPLGRGAFGQVIEADAFGIDKTATCRTVAVKMLKEGATHSEHRALMS | |
| | ELKILIHIGHHLNVVNLLGACTKPGGPLMVIVEFCKFGNLSTYLRSKRNEFVPYKTKGAR | |
| | FRQGKDYVGAIPVDLKRRLDSITSSQSSASSGFVEEKSLSDVEEEEAPEDLYKLFLTLEH | |
| | LICYSFQVAKGMEFLASRKCIHRDLAARNILLSEKNVVKICDFGLARDIYKDPDYVRKGD | |
| | ARLPLKWMAFETIFDRVYTIQSDVWSFGVLLWEIFSLGASPYPGVKIDEEFCRRLKEGTR | |
| | MRAPDYTTPEMYQTMLDCWHGEPSQRPTFSELVEHLGNLLQMJAQQDGKLYIVLPISETL | |
| | SMEEDSGLSLPTSPVSCMEEEEVCDPKFHYDNTAGISQYLQNSKRKSRPVSVKTFEDIFL | |
| | EEPEVKVIPDDNQTDSGMVLASEELKTLEDRTKLSPSFGGMVPSKSRESVASEGSNQTSG | |
| | YQSGYHSDDTDTTVYSSEEAELLKLIEIGVQTGSTAQILQPDSGTTLSSPPV | |

Wild-type granzyme B proteases and variants have been successfully expressed as active proteases in yeast or bacterial expression systems at multi-milligram quantities. See, e.g., protocols described in Harris 1998; Friedrich, 2002; and Takeuchi, 2000. Granzyme B was engineered to obtain muteins that exclusively cleave Flk-1/KDR. CB01 (I99A/N218A), CB02 (I99A/N218A/L171E), CB05(I99A/N218A/K172D), CB06 (I99A/N218A/Y174E), CB07 (I99A/N218A/Y174D) and CB10 (I99A/N218A/R192M) are first generation results of this work with novel extended specificity with modified substrate selectivity at the P2 through P4 positions.

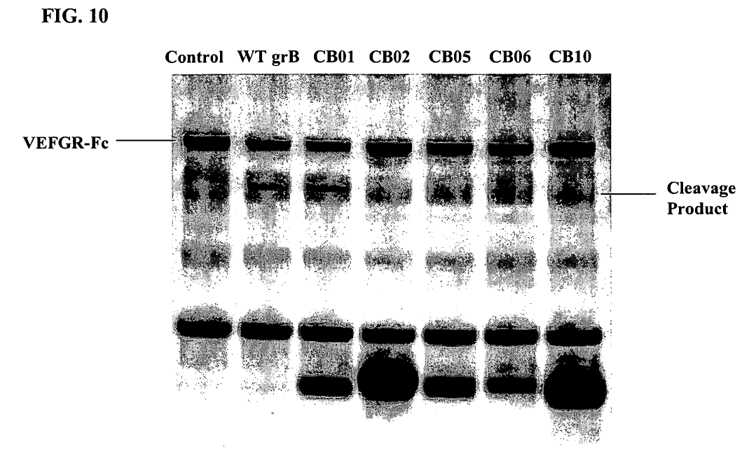

These variants and the wild-type granzyme B proteases were evaluated for their ability to catalyze in vitro cleavage of a soluble Flk1-Fc fusion protein consisting of the entire Flk1 ectodomain fused to an IgG2a antibody Fc fragment. Wild-type granzyme B and the listed muteins cleave the VEGFR-Fc receptor (FIG. 10). These preliminary in vitro experiments confirm that variant proteases will cut the receptor at specific sequences and that the identified target sequences are accessible to proteolysis. The cleavage site of CB01 and wild-type granzyme B were identified from the gel by the Edman N-terminal sequencing method. Flk1-Fc is cleaved by wild-type and granzyme B muteins at the sequence LVED/SGID

### Example 6

### Assaying Cleavage of Purified VEGF Receptor

Purified extracellular domain of VEGF-R2 fused to the Fc domain of mouse IgG (3-10 μg) is resuspended in granzyme activation buffer (20 μL). Variant proteases are added to a final concentration of 100 nM to 1 μM. The reaction is incubated at 37° C. for 1-2 hours and then separated by SDS-PAGE electrophoresis. Bands are visualized by Coomassie blue staining, silver staining, and/or Western blot.

Result. The purified VEGFR2-Fc is efficiently cleaved by wild-type and mutant granzyme B (FIG. 10). Cleavage by variant proteases yields cleavage products with apparent molecular weights of ˜80 kDa and 30 kDa; analysis of potential cleavage sites in VEGFR2 suggests that variants target the stalk (membrane proximal) region of VEGFR2. All mutants cleave full-length VEGFR2, some at a reduced rate compared to the wild-type, while others cleave the receptor with higher efficiency than wild-type. None of the protease variants (wild-type or mutant) cleave the Fc domain.

### Example 7

### Granzyme B is Secreted in an Active Form from Mammalian Cells

Human embryonic kidney cells (HEK293T) were grown to 95% confluence in 24-well please. DNA constructs encoding the granzyme B protease domain fused to Fc with a N-terminal IgK leader sequence were transfected using Lipofectamine 2000 (Invitrogen) according to the manufacturer's protocol. After 40 hours, 100 μL of the medial were collected from each well, to which was added a fluorogeneic granzyme B substrate (Ac-IETD-AMC, BaChem) to a final concentration of 200 μM. Granzyme B activity was detected using a Gemini XS fluorescence plate reader (Molecular Devices) with an excitation wavelength of 380 nm and an emission wavelength of 450 nm.

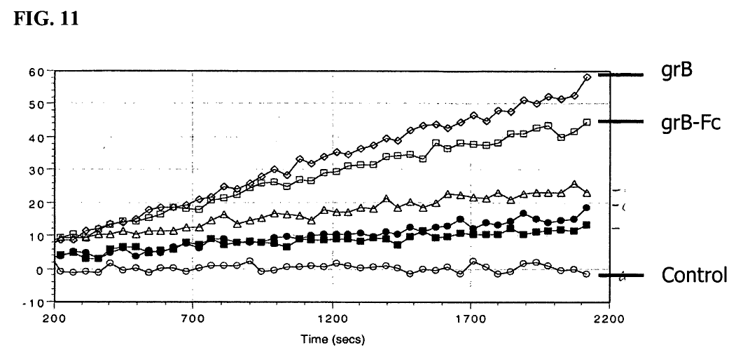

Result: As shown in FIG. 11, both granzyme B and grB-Fc are secreted in active forms from HEK293T cells, as evidenced by granzyme B- specific cleavage of a substrate. There is no activity in the control wells where there was no DNA added, suggesting that HEK293T cells normally do not make and secrete granzyme B. In addition, since the cells were grown in media containing 10% fetal calf serum, the fact that granzyme B was found to be active in this media suggests that macromolecular inhibitors present in serum are not effective at inhibiting granzyme B. These data suggest that granzyme B can be delivered into animals by adenovirus and that the adenoviral encoded protease can be secreted and is active in serum.

### Example 8

### Microcorneal Pocket Model

Rat granzyme B was cloned into pAdd or pAdd-Fc vectors for adenoviral delivery in mice according to Kuo et al., PNAS, 2001, 98:4605-4610.

C57BL/6 mice receive 5×10⁸ pfu i.v. of protease-encoding adenoviruses or the control adenovirus Ad Fc 2 days before assay. Mice are anesthetized with avertin i.p. and the eye was treated with topical proparacaine HCl (Allergan, Irvine, Calif.). Hydron/sucralfate pellets containing VEGF-A₁₆₅ (R & D Systems) are implanted into a corneal micropocket at 1 mm from the limbus of both eyes under an operating microscope (Zeiss) followed by intrastomal linear keratotomy by using a microknife (Medtroni Xomed, Jacksonville, Fla.). A corneal micropocket is dissected toward the limbus with a von Graefe knife #3 (2×30 mm), followed by pellet implantation and application of topical erythromycin. After 5 days, neovascularization is quantitated by using a slit lamp biomicroscope and the formula 2π×(vessel length/10)×(clock hours). P values are determined by using a two-tailed t test assuming unequal variances (Microsoft EXCEL).

To detect the expression and secretion virally encoded granzyme B in serum, mice were bled at varying time points over the course of 3 weeks. Two microliters of serum was separated by SDS-PAGE, transferred to nitrocellulose membrane, and probed with a polyclonal antibody specific to granzyme B.

Results. Injection of adenovirus encoding granzyme B into mice resulted in robust expression and secretion of the protease into serum, with maximal expression occurring on day 2 and extending until day 5 (FIG. 13A). The production of granzyme B resulted in 60% inhibition of VEGF-induced neovascularization when tested in the cornea micropocket model as compared to animal treated with control virus (FIG. 13B). These data validate the use of granzyme B as an anti-angiogenic agent in an in vivo model of neovascularization.

### Example 9

### Tumor Xenograph Model

Murine Lewis lung carcinoma (LLC) cells are passaged on the dorsal midline of C57BL/6 mice or in DMEM/10% FCS/penicillin/streptomycin(PNS)/L-glutamine. T241 murine fibrosarcoma is grown in DMEM/10% FCS/PNS/L-glutamine and human pancreatic BxPc3 adenocarcinoma in RPMI medium 1640/10% FCS/PNS. Tumor cells (10⁶) are injected s.c. into the dorsal midline of C57BL/6 mice (8-10 weeks old) for murine tumors and severe combined immunodeficient (SCID) mice for human tumors, grown to 100-200 mm³ (typically 10-14 days) to demonstrate tumor take, and 10⁹ pfu of protease-encoding adenoviruses or the control adenovirus Ad Fc given by i.v. tail-vein injection. Tumor size in mm³ is calculated by caliper measurements over a 10- to 14-day period by using the formula 0.52×length (mm)×width (mm), using width as the smaller dimension. See, e.g., Kuo et al., PNAS, 2001, 98:4605-4610. P values were determined by using a two-tailed t test assuming unequal variances (Microsoft EXCEL).

Results. Given that cleavage of VEGFR2 will inactivate the receptor, then the systemic delivery of therapeutically effective amounts of protease—either as purified protein or encoded by adenovirus—will result in inhibition of LLC tumor growth. Failure to inhibit tumor growth may be due to the inactivation of the protease by endogenous protease inhibitors (serpins). In such an event, the covalent binding of the serpin to the protease will be detectable as an increase in size of the protease by SDS-PAGE. Mutations can be made in the protease that will make it resistant to serpin inactivation.

### Example 10

### Use of Ecotin for Defining an Active Site

The invention provides variants of the macromolecular protease inhibitor ecotin that are mated to the engineered proteases for defining the active site. Ecotin is a powerful tool for defining the active sites of serine protease due to the extended substrate-like interaction that it makes with the protease. It takes a molecular impression of the active site revealing features of substrate preference. The 80's loop of ecotin binds to the protease in an extended beta sheet conformation. Site directed mutagenesis is used to mutate amino acids 81 through 86 to match the substrate specificity of interest. For each variant protease, an ecotin variant is made that matches the extended specificity in the P1 through P4 positions (residues 81 through 84 of ecotin). The inhibition constant (Kᵢ) of the protease/inhibitor complex is then measured using protocols established in the art and derived from tight binding equations.

Each protease is measured with a specific fluorogenic tetrapeptide substrate that matches the proteases specificity of the variant mutein, and with an ecotin that matches the specificity and the target sequence. In earlier studies in the art using uPA, some ecotins with altered reactive sites loops did not form tight complexes with the substrate and instead were proteolyzed. If this occurs, a tetrapeptide chloromethyl ketone inhibitors is provided with matching specificity for the crystallography.

### Example 11

### Crystallization and Determination of 3-Dimensional Complex Structures

Structural studies of the tetrameric complex between the mated protease and ecotin are performed. Ecotin binds to and inhibits proteases with a broad range of pI values forming an E₂P₂ complex that typically buries between 1900 and 2500 ○². This large and strongly associated complex is able to form crystals at a range of pH values from pH 5.0 to pH 8.8.

To form the E₂P₂ complex an equimolar mixture of protease and ecotin is made, complexed for 1 hour and purified by column chromatography to separate free protease from the complex. With this additional purification step, no protease-ecotin complex has failed to crystallize to date. Structural studies must reveal side chain orientations and solute and solvent binding to be effective. The minimum resolution of X-ray data needed for is about 2.3-2.5 Å, a resolution that is usually achieved. In some cases, 2.18 to 2.0 Å data will be useful to determine whether critical side chains show multiple positions. Data will be collected at a synchrotron ALS Beamline 8.3.1.

Once a crystallization condition has been optimized for each wild-type protease and ecotin, it should remain similar for every mutant, since crystal contacts are distant from the active site and between invariant protease-protease contacts or ecotin-ecotin contacts. The similarity between complexes will also aid in structure determination. Molecular replacement within the wild-type complex of granzyme B-ecotin will be used to solve the structure for the muteins.

Structural data on engineered proteases complexed with target-like peptides provide a framework to understanding direct and second shell side chain interactions that proscribe specificity. This correlation of three dimensional structure and protease activity and specificity, are of clinical and academic interest.

### Example 12

### Preparation and Storage of I99A Granzyme B

The wild-type rat granzyme B construct was prepared as described previously (Harris et al., JBC, 1998, (273):27364-27373). The following point mutations were introduced into the pPICZαA plasmid: N218A, N218T, N218V, I99A, I99F, I99R, Y174A, Y174V. Each mutation was confirmed by sequencing with primers to the 5′AOX and 3′AOX regions, followed by transformation into X33 cells and selection with Zeocin (Invitrogen, La Jolla Calif.). Expression and purification for each variant was identical to the previously described method for wild-type rat granzyme B (Harris, et al., JBC, 1998, (273):27364-27373).

The protease rat granzyme B was mutated at Ile 99 to an Alanine using the QuikChange (Stratagene) method of site directed mutagenesis. DNA primers to introduce the I99A mutation were: Forward primer: CCA GCG TAT AAT TCT AAG ACA GCC TCC AAT GAC ATC ATG CTG (SEQ ID NO:30) Reverse primer: CAG CAT GAT GTC ATT GGA GGC TGT CTT AGA ATT ATA CGC TGG (SEQ ID NO:31). A polymerase chain reaction was made containing the wild-type double stranded DNA, the two primers overlapping the mutation, a reaction buffer, dNTP's and the DNA polymerase. After 30 rounds of annealing and amplification, the reaction was stopped. The enzyme DpnI was added to digest the wild-type DNA containing a modified base pair, and the resulting nicked DNA strand is transformed into bacteria. A selection against Zeocin ensures only positive clones with grow. The mutation was confirmed by sequencing the granzyme B gene. The same protocol was used to make the remaining granzyme B mutants, with appropriate changes in the mutagenic primers.

The DNA containing the variant granzyme B proteases was transformed into Pichia pastoris X33 cells by the published protocol (Invitrogen) and the positive transformants were selected with Zeocin. The colony was transferred to a 1 L liquid culture and grown to a cell density of greater than OD600=1.0. Protein expression was induced by the addition of 0.5% methanol and held constant over 72 hours. To purify the variant protease, the culture was centrifuged and the supernatant collected. Gravity based loading flowed the supernatant over a SP-Sepharose Fast Flow cation exchange column. The column was washed with 50 mM Mes, pH 6.0, 100 mM NaCl, and more stringently with 50 mM MES, pH 6.0, 250 mM NaCl. The protein was eluted with 50 mM MES, pH 6.0, 1 M NaCl and the column washed with 50 mM MES, pH 6.0, 2M NaCl and 0.5 M NaOH. The resulting protease was <90% pure. The final protease was exchanged and concentrated into 50 mM MES, pH 6.0, 100 mM NaCl for storage at 4° C.

Alternatively, following purification, each variant was quantitated by absorbance at 280 nm (e280=13000 M-1cm-1), titrated with wild-type ecotin or M84D ecotin as previously described, exchanged into a buffer of 50 mM MES, pH 6.0 and 100 mM NaCl and stored at 4° C.

### Example 14

### Screening for Cleavage of Individual Substrates

Mutant proteases that match the desired specificity profiles, i.e. cleave a sequence present in VEGFR as determined by substrate libraries, are assayed using individual peptide substrates corresponding to the desired cleavage sequence. Individual kinetic measurements are performed using a Spectra-Max Delta fluorimeter (Molecular Devices). Each protease is diluted to between 50 nM and 1 μM in assay buffer. All ACC substrates are diluted with MeSO to between 5 and 500 μM, while AMC substrates are diluted to between 20 and 2000 μM. Each assay contain less than 5% MeSO. Enzymatic activity is monitored every 15 seconds at excitation and emission wavelengths of 380 mn and 460 nm, respectively, for a total of 10 minutes. All assays are performed in 1% DMSO.

### Example 15

### Screening for Cleavage of Full-Length Proteins

Variant proteases are assayed to ascertain that they will cleave the desired sequence when presented in the context of the full-length protein, and the activity of the target protein is assayed to verify that its function has been destroyed by the cleavage event. The cleavage event is monitored by SDS-PAGE after incubating the purified full-length protein with the variant protease. The protein is visualized using standard Coomasie blue staining, by autoradiography using radio labeled protein, or by Western blot using the appropriate antibody. Alternatively, if the target protein is a cell surface receptor, cells expressing the target protein are exposed to the variant protease. The cleavage event is monitored by lysing the cells and then separating the proteins by SDS-PAGE, followed by visualization by Western blot. Alternatively, the soluble receptor released by proteolysis is quantified by ELISA.

Cleavage Of VEGFR.

¹²⁵I-VEGFR (40,000 cpm) is incubated with varying concentrations of protease, samples are boiled in SDS-PAGE sample buffer and examined on a 12% polyacrylamide gel. The gels are dried and exposed to x-ray film (Kodak) at −70° C.

VEGFR Binding Assay.

¹²⁵I-VEGFR or PMN are incubated with varying concentrations of proteases as above. The binding of ¹²⁵I-VEGFR exposed to proteases to normal PMN, or the binding of normal ¹²⁵I-VEGFR to PMN exposed to proteases, are quantified using scintillation. Briefly, 10⁵ cells are incubated with varying concentrations of ¹²⁵I- VEGFR in 96-well filter plates (Millipore) in the presence of protease inhibitors. Cells are washed three times by vacuum aspiration and 30 μL of scintillation fluid (Wallac) are added to each well. Scintillation are counted on a Wallac Microbeta scintillation counter. (Adapted from van Kessel et al., J. Immunol. (1991) 147: 3862-3868 and Porteau et al., JBC (1991) 266:18846-18853).

### Example 16

### Characterizing Structural Determinants of Granzyme B

Cytotoxic lymphocytes are the major immune system defense against virally and tumor infected cells. They efficiently initiate apoptosis in the target cell by the directional release of cytotoxic granules. Within these granules is a family of serine proteases known as the granzymes along with the pore forming protein perforin and serglycan. The granzymes are a family of trypsin-like serine proteases and thus are composed of two β barrel domains that bind a peptide substrate in an extended β strand interaction composed of at least four backbone hydrogen bonds. They are expressed as pre-pro-proteases where the pre region targets them to the granules, and activation occurs through the proteolytic processing of the pro region by dipeptidyl protease 1. Of particular interest in this family is granzyme B, a well characterized serine protease with specificity for aspartic acid that is unique among serine proteases.

Native granzyme B initiates the apoptotic cascade by cleaving caspases 3 and 7 and contributes to cytoplasmic and nuclear hallmarks of cell death by proteolyzing a specific set of intercellular substrates. The strict requirement by granzyme B for an aspartic acid residue at the primary, or P1 position is unique among mammalian enzymes as is the requirement for a specific extended substrate containing Ile/Val at the P4 position, Glu/Gln/Met at the P3 position, any amino acid at the P2 position, non-charged amino acids at the P1′ position and Ser/Ala/Gly at the P2′ position.

It is located in a tightly linked gene cluster on chromosome 14 of the rat, mouse and human species along with chymase, cathepsin G, rat mast cell protease and granzymes C through H. This subfamily of serine proteases are homologous, sharing >46% identity and >56% similarity, yet they have at least five identified or predicted primary specificities and unique extended substrates. The narrow substrate specificity of such regulatory serine proteases is thought to occur through a constellation of amino acids identified by x-ray crystallography surrounding the active site. The specific interactions that determine narrow extended substrate specificity occur between substrate and protease side chains on the highly variable loops between the P strands.

In trypsin-fold serine proteases the primary site amino acids 189, 191, 216, and 226 and the loops containing them have been investigated through protein engineering for their roles in P1 specificity in other serine proteases. They demonstrate that the S1 subsite in serine proteases is usually optimized for specific recognition of a single or perhaps two amino acids. All of the granzyme B-like serine protease are missing a disulfide bond between amino acids 191 and 220 and have a truncated 220's loop containing a cis-Pro. The primary S1 specificity determinant is located at amino acid 226. In granzyme B, amino acid 226 is Arg and by three-dimensional structure analysis makes a salt bridge interaction with the P1-Asp of the substrate. Mutating this amino acid to Asp results in a protease with P1 basic specificity against ester substrates. Successful efforts to change trypsin basic specificity to chymotrypsin aromatic specificity required grafting entire active site loops and altering second shell interactions. This effort is perhaps less difficult in the granzyme B-like proteases as they share a S1 pocket architecture that exhibits remarkable accommodation by subtle alterations distant in three dimensions. S1 has evolved to accommodate the chymase preference for aromatic amino acids Phe and Tyr, the cathepsin G dual specificity for basic and aromatic amino acids and the granzyme B preference for aspartic acid.

Previous experiments on trypsin-like proteases show amino acids 192 and 99 are responsible for differences in the substrate selectivity and inhibitor binding of activated protein C, thrombin and Factor Xa. Thus, the high sequence identity of the granzyme B-like subfamily narrows the number of possible extended specificity determinants, and suggests that mutagenic changes will introduce radical alterations in extended specificity. The coincidence of crystal structures of granzyme B, chymase, rat mast cell protease and cathepsin G and combinatorial assays for determining substrate specificity provide a powerful basis for understanding extended substrate specificity using site-directed mutagenesis. This study seeks to understand the contribution of amino acids identified by the crystal structure of rat granzyme B to its extended specificity, and the role of mutations in the selective modification of its specificity. The contributions of subfamily specific amino acids, were probed with a series of variants constructed along the active site binding cleft. Eight single and three double mutation variants were designed with substitutions at Tyr174, Ile99, Arg192 and Asn218. A facile method to determine the P1 to P4 specificity of an enzyme is represented by positional scanning synthetic combinatorial libraries (PSSCL) of substrates. We used a P1-Asp PSSCL to screen each variant for alterations in P2 though P4 specificity and designed individual substrates to probe the extent of novel specificities through Michealis-Menton kinetics.

Experimental Procedures

Materials

Ac-IEPD-AMC (SEQ ID NO:32), Ac-IKPD-AMC (SEQ ID NO:33), Ac-IETD-AMC (SEQ ID NO:34), and Ac-AAD-pNA were purchased from Sigma (St. Louis, Mo.). Ac-IEFD-AMC (SEQ ID NO:35), Ac-LEFD-AMC (SEQ ID NO:36), and Ac-LEPD-AMC (SEQ ID NO:37)were purchased from SynPep (Dublin, Calif.). The QuikChange mutagenesis kit was purchased from Stratagene (La Jolla, Calif.) and used according to manufacturer's instructions. All oligonucleotide primers and the P. pastoris expression system were purchased from Invitrogen (San Diego, Calif.). The Asp-ACC conjugated resin was a gift from the lab of John Ellman (UC Berkeley). The P1-Asp-AMC library was a gift of Nancy Thornberry (Merck Laboratories, Rahway, N.J.). The complete diverse ACC tetrapeptide substrate library was a gift of Youngchool Choe (Craik Lab).

Alignment of Granzyme B Sub-Family Serine Proteases

Rat granzyme B was used as a query to find related granzyme and granule proteases from the NCBI database (National Library of Medicine, National Institutes of Health, Bethesda, Md.) using PSI-BLAST and HSSP. The sequences were aligned using ClustalW 8.1. The amino acid sequence of rat granzyme B is found under the NCBI Accession # NP_{—}612526 or the Swiss-Prot # 18291. The x-ray crystallographic structure of rat granzyme B complexed with ecotin [81-84 IEPD] was used to determine amino acids within 4 Å of the substrate amino acids. Amino acids were also evaluated that contact small molecule inhibitors in the human granzyme B, chymase, cathepsin G and rat mast cell protease II structures. The atomic coordinates of the analyzed structures are available from the RCSB Protein Databank under the following identification numbers: rat granzyme B=PDB # 1F18, human granzyme B=PDB # 1FQ3 and 11AU, chymase=PDB # 1PJP and 1KLT, cathepsin G=PDB # 1AU8 and 1CGH, and rat mast cell protease II=PDB # 3RP2.

Variants of Rat Granzyme B

The wild-type, R192A, and R192E rat granzyme B constructs were prepared as described above. Additional variants of wild-type rat granzyme B were constructed using the QuikChange protocol (Stratagene, La Jolla, Calif.). The following 5′ to 3′ primers were made to introduce point mutations into the pPICZαA plasmid. Mutations are underlined. N218A: GGC ATC GTC TCC TAT GGA CAA GCT GAT GGT TCA ACT CCA CGG GCA (SEQ ID NO:37), I99A: CCA GCG TAT AAT TCT AAG ACA GCC TCC AAT GAC ATC ATG CTG (SEQ ID NO:38), I99F: CCA GCG TAT AAT TCT AAG ACA TTC TCC AAT GAC ATC ATG CTG (SEQ ID NO:39), I99R: CCA GCG TAT AAT TCT AAG ACA AGA TCC AAT GAC ATC ATG CTG (SEQ ID NO:40), Y174A: GT GAG TCC TAC TTA AAA AAT GCT TTC GAC AAA GCC AAT GAG ATA (SEQ ID NO:41). Each mutation was confirmed by sequencing with primers to the 5′AOX and 3′AOX regions. The resulting plasmid was transformed into X33 cells and selected with Zeocin (Invitrogen, La Jolla Calif.). All granzyme B variants were expressed and purified to homogeneity by the wild-type protocol with yields between 0.5 and 3 mg/L. Activity was monitored in the supernatant using 1 μM Ac-AAD-pNA during expression. Following purification each variant was exchanged into a buffer containing 50 mM MES pH 6.0 and 100 mM NaCl, quantified by absorbance at 280 nm (A₂₈₀=22900 M⁻¹cm⁻¹), titrated with wild-type or M84D ecotin, and stored at 4° C.

Positional Scanning Synthetic Combinatorial Libraries.

Both PSSCL were measured into Microfluor black 96-well plates (DYNEX Technologies, Chantilly, Va.) and diluted to a final concentration of 250 μM. Thus the tetrapeptide substrates in each pool are at a concentration far below the expected Kₘ of the variant protease, and the initial rates are proportional to the specificity constant k_{cat}/Kₘ. The granzyme B variants were assayed for activity against Ac-IEPD-AMC (SEQ ID NO:32), and diluted in granzyme activity buffer (50 mM Na HEPES, pH 8.0, 100 mM NaCl, 0.01% Tween-20) to concentrations between 50 nM and 1 μM that yielded PSSCL activity levels approximately equal to 50 nM wild-type granzyme B. Initially, each variant was assayed in the P1 sub-library of a tetrapeptide completely diverse PSSCL (Youngchool Choe, in preparation). It consists of 20 spatial arrayed pools of 8000 fluorogenic tetrapeptide ACC substrates where the P1 amino acid is fixed (cystine was deleted and norleucine is included) while the P2, P3 and P4 amino acids consist of equimolar amino acid mixtures. To increase the signal to noise ratio, and reduce the number of assayed peptide substrates, the P1-Asp aminomethyl coumarin (AMC) based PSSCL used to profile caspases and human and rat granzyme B was used to measure extended (P2 through P4) selectivity. Each sub-library consists of 19 pools containing 361 peptides, one each for the spatially addressed amino acids (cystine and methionine are deleted and D-alanine is included). Enzymatic activity in the PSSCL was assayed for one hour at 30° C. on a SpectraMAX Gemini fluorimetric plate reader (Molecular Devices Corporation, Sunnyvale, Calif.) and the rate of substrate hydrolysis analyzed with the SOFTmax PRO software (version 3.1.1 Molecular Devices Corporation). Excitation and emission were measured at 380 nm and 450 nm, respectively.

Synthesis of ACC Substrates

Rink resin conjugated ACC-Asp(OtBu)-Fmoc was subjected to standard solid phase peptide synthesis, acylated and cleaved by trifluoroacetic acid. The resulting substrates were purified to homogeneity by reverse phase HPLC chromatography on a C18 column (Vydac, 5 μm, 4.6×250 mm) with a 20-60% gradient of 0.1% aqueous trifluoroacetic acid and 0.08% trifluoroacetic acid, 95% acetonitrile. The molecular weight of each substrate was confirmed by MALDI mass spectrometry. The concentration of each substrate stock solution was verified by total hydrolysis of the ACC or AMC leaving group using saturating amounts of wild-type granzyme B. Briefly, a measured amount of each substrate was exposed to saturating amounts of enzyme, and the total fluorescence measured over multiple days until all of the substrate was hydrolyzed.

Individual Kinetic Measurements

Individual kinetic measurements were performed using a SpectraMAX Gemini fluorometric plate reader. Each protease was diluted to between 50 nM and 1 μM in assay buffer. All ACC substrates were diluted in DMSO to between 5 and 500 μM, while AMC substrates were diluted to between 20 and 2000 μM. Each assay contained less than 5% (v/v) DMSO. Enzymatic activity was monitored every 15 seconds at excitation and emission wavelengths of 380 nm and 460 nm, respectively, for a total of 10 minutes. Rates of substrate hydrolysis were determined using the SOFTmax PRO data analysis software (version 3.1.1, Molecular Devices Corp.) and fit to the Michaelis-Menten equation using Kaleidagraph (Version 3.5, Synergy Software, Reading, Pa.). When the apparent Kₘ values of for a substrate was greater than 2.2 μM, only k_{cat}/Kₘ was reported. The substrates were assayed for hydrolysis in duplicate at least twice by granzyme B and variants.

Results

Potential Extended Specificity Determinants are Limited by Structural Homology

From the x-ray crystallographic structure of rat granzyme B with the pseudo-substrate Eco [81-84 IEPD (SEQ ID NO:32)], amino acid side chains within 4 Å of side chains of the ecotin binding-site loop were determined (FIGS. 5E-5F). They include Phe191 and Arg226 in the S1 pocket, Ile99 and His57 in the S2 pocket, Arg192 and Asn218 in the S3 pocket, and Gln180, Phe174, Tyr215, and Leu172 in the S4 pocket. The 23 members of the granzyme B-like subfamily of serine proteases were aligned and the structural determinants identified in the primary sequences (Table 2). Amino acid 226 has been identified as the primary determinant of P1 specificity, and the determinants His 57, Leu172, Phe 191, Gln 180 and Tyr 215 vary between two or fewer amino acids in the subfamily or are absolutely required for catalytic activity. Thus many potential determinants of specificity from crystallographic studies could be excluded from mutagenic studies because of known structural characteristics independent of extended specificity. Of the identified determinants, Ile99, Asn218, Arg192, and Tyr174 were altered with site-directed mutagenesis. The variants were single mutants I99A, I99R, I99F, N218A, N218T and Y174A and double mutants N218A/I99A, N218A/R192A and N218A/R192E. Initial activity in the complete diverse PSSCL indicated that all granzyme B variants specifically and uniquely hydrolyzed substrates containing P1 aspartic acid. This result is consistent with the strict specificity of granzyme B for aspartic acid.

PSSCL Reveals Extended Specificity Changes Upon Alanine Mutagenesis

Each alanine variant was profiled with the P1-Asp AMC PSSCL. Granzyme B I99A had a large effect on the PSSCL specificity profiles at the P2 position. The mutation increased the hydrolysis of P2-Phe or Tyr substrates and reduced the hydrolysis of non-hydrophobic side chains without altering the P3 or P4 profiles compared to wild-type granzyme B (FIG. 1, Table 21). Activity for the P2 amino acid Phe was nine times the activity for Pro compared to a 3.5 fold P2-Pro preference for wild-type granzyme B. The novel hydrophobic preference also extends to tetrapeptide substrates. The specificity constant, k_{cat}/Kₘ, for Ac-IEPD-AMC and Ac-IEFD-AMC were 57 M⁻¹s⁻¹ and 268 M⁻¹s⁻¹, respectively, a five fold difference in activity. Yet, these kinetic constants represent a >10 fold reduction in the overall activity of the wild-type protease. The preference for Phe and Tyr is increased but in the context of a drop in activity reflected in an eight fold decrease in k_{cat} (from 1.19 s⁻¹ to 0.15 s⁻¹).

Two positions restrict specificity at P3. Asn218 was replaced with Ala to probe the role of the hydrogen bond observed between the amino group of Asn218 and the carboxyl group of the ecotin Glu83 in the x-ray crystal structure. N218A granzyme B did not change the P2 or P4 specificity profiles, but decreased the preference for PSSCL substrates with Glu at the P3 position, broadened the number of accepted P3 amino acids to include Ser and Ala, and increased the preference for Met and Gln (FIG. 1, Table 20). A similar result was observed for the R192K and R192A mutations to granzyme B. Hydrolysis of Ac-IKPD-AMC by N218A granzyme B was increased >2 fold compared to wild-type activity from 99 M⁻¹s⁻¹ to 220 M⁻¹s⁻¹ whereas, hydrolysis of Ac-IEPD-AMC was reduced from 3300 M-1s-1 to 2200 M⁻¹s⁻¹. The change in activity was observed in both the k_{cat} and Kₘ of the N218A variant (Table 20). The decrease in preference for Ac-IEPD-AMC was accompanied by an increase in the preference for Ala by 2.5 times.

The Y174A granzyme B variant exhibited no alteration in the PSSCL profile preferring the canonical P4-Ile, P3-Glu, and P2-Pro sequence (FIG. 1, Table 20). Instead Y174A granzyme B had activity approximately 10 fold less than the wild-type activity (470 M⁻¹s-⁻¹ versus 3300 M⁻¹s⁻¹). The decrease in activity was due to a decrease in the k_{cat} value from 1.19 s⁻¹ to 0.87 s⁻¹ and an increase in the Kₘ from 370 μM to 1000 μM. This reduction in activity with no change is specificity suggests that the phenyl side chain acts to stabilize the transition state and orient the scissile bond but has little effect on the identity of the P4 amino acid.

Table 20 shows the kinetic parameters for the hydrolysis of the individual tetrapeptide substrates Ac-IEPD-AMC (SEQ ID NO:32), Ac-IEFD-AMC (SEQ ID NO:35), Ac-IAPD-AMC (SEQ ID NO:42) and Ac-IKPD-AMC (SEQ ID NO:33), by the indicated Granzyme B protease. The highlighted amino acids designate the target residues that deviate from the preferred wild-type substrate.

TABLE 20
Granzyme B wild-type, I99A, Y174A and N218A mutein
specificity.

| | | | |
|---|---|---|---|
| | k_{cat}/Kₘ | k_{cat} | Kₘ |
| granzyme B | M⁻¹ sec⁻¹ | sec⁻¹ | μM |

WT

| | | | |
|---|---|---|---|
| Ac-IEPD-AMC | 3300 ± 500 | 1.2 ± 0.033 | 370 ± 70 |
| Ac-IEFD-AMC | 1000 ± 99 | 0.15 ± 0.003 | 153 ± 34 |
| Ac-IAPD-ACC | 110 ± 5 | N.D. | N.D. |
| Ac-IKPD-AMC | 99 ± 5 | 0.15 ± 0.01 | 1600 ± 190 |

I99A

| | | | |
|---|---|---|---|
| Ac-IEPD-AMC | 57 ± 2.6 | 0.14 ± 0.016 | 2500 ± 320 |
| Ac-IEFD-AMC | 270 ± 18 | 0.24 ± .010 | 880 ± 96 |

Y174A

| | | | |
|---|---|---|---|
| Ac-IEPD-AMC | 470 ± 36 | 0.87 ± 0.03 | 1000 ± 120 |

N218A

| | | | |
|---|---|---|---|
| Ac-IEPD-AMC | 2200 ± 190 | 1.6 ± 0.09 | 700 ± 94 |
| Ac-IKPD-AMC | 220 ± 19 | 0.38 ± 0.09 | 1800 ± 550 |

Variations Based on Family Homology Modulate Activity

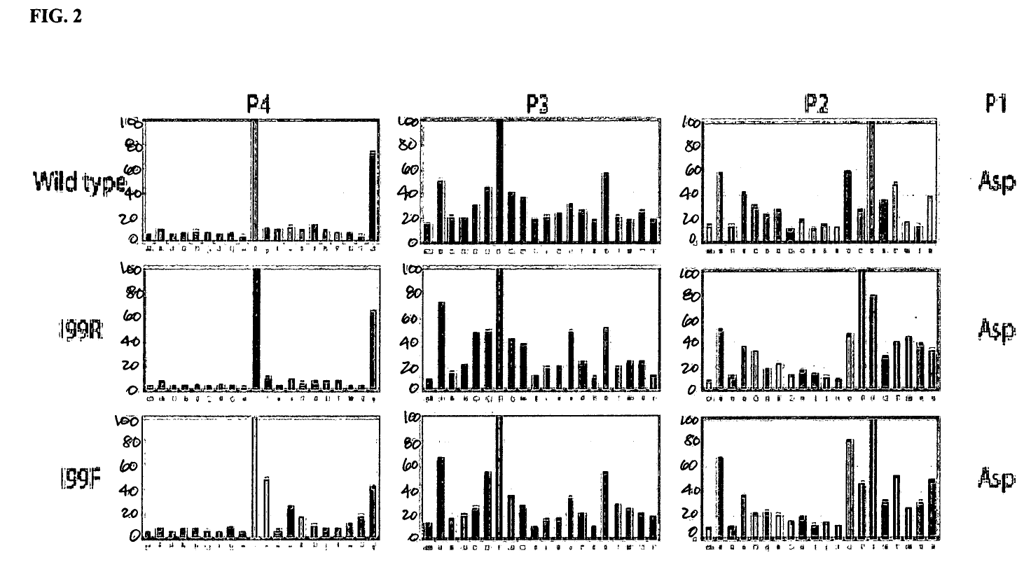

The I99R, I99F, and N218T granzyme B variants were designed to test structural determinants found in the granzyme subfamily. Phe-99 occurs in the human homolog of rat granzyme B, and Arg-99 is found in granzymes A and C (FIG. 2, Table 21). The I99F variant prefers the wild-type granzyme B substrate sequence Ile-Glu-Pro by the PSSCL, but the specificity for P4-Leu increases. The preference for P4-Ile versus P4-Leu is 2.6 fold as measured with tetrapeptide substrates for I99F granzyme B versus 370 fold for wild-type granzyme B (Table 21). The specificity constant of I99F granzyme B towards Ac-IEPD-AMC, 1100 M⁻¹s⁻¹, is less effected by the mutation. A decrease in the k_{cat}, from 1.19 s⁻¹ to 0.56 s⁻¹ accounts for much of the reduction in activity. In the variant I99R, a hydrophobic preference at P2 appears, but it is less apparent than the I99A variant. Pro and Phe substrates are hydrolyzed at similar rates (k_{cat}/Kₘ is 330 M⁻¹s⁻¹, 505 M⁻¹s⁻¹, respectively). The primary differentiation is in the Kₘ values. The Kₘ of the P2-Phe substrate (980 μM) is less than the Kₘ for the wild-type P2-Pro substrate (1800 μM), while the rates are equivalent (k_{cat} is 0.47 s⁻¹ and 0.55 s⁻¹, respectively).

Asn218 faces the active site cleft and forms a hydrogen bond with the carboxylate group of Glu82 in ecotin. This central location arises from the truncation of the 220's loop compared to trypsin, and the kink induced by a cis-Pro at position 217. Asn218 was replaced with Thr, to probe the role of this hydrogen bond. N218T decreased the preference for the PSSCL substrates with Glu at the P3 position, broadened the number of accepted P3 amino acids to include Ser and Ala, and increased the preference for Met and Gln (FIG. 2, Table 13). The mutations did not change the P2 or P4 specificity. Ala was the most preferred P3 amino acid, and the preference for amino acids such as Lys remained low (k_{cat}/Kₘ 600 M⁻¹s⁻¹).

Kinetic rate constants for the individual tetrapeptide substrates Ac-IEPD-AMC (SEQ ID NO:32), Ac-IETD-AMC (SEQ ID NO:34), Ac-LEFD-AMC (SEQ ID NO:36) and Ac-LEPD-AMC (SEQ ID NO:37) illustrate the magnitudes of the altered specificity where the highlighted amino acids deviate from the preferred wild-type substrate sequence. See Table 21. The presented ratios are of the wild-type preference (P) to the variant's novel preference (X). The novel preference (X) is the bold amino acid of the substrate. Ratios are shown for activity in the PSSCL libraries (PSSCL) and between individual tetrapeptide substrates (Activity).

TABLE 21
Granzyme B wild-type, I99F, I99R and N218T mutein specificity.

| | | | | | |
|---|---|---|---|---|---|
| | K_{cat}/Kₘ | K_{cat} | Kₘ | PSSCL | Activity |
| granzyme B | M⁻¹ sec⁻¹ | sec⁻¹ | μM | Ratio (P:X) | Ratio (P:X) |

WT

| | | | | | |
|---|---|---|---|---|---|
| Ac-IEPD-AMC | 3300 ± 500 | 1.2 ± 0.033 | 370 ± 70 | | |
| Ac-IETD-AMC | 1600 ± 260 | 0.39 ± 0.02 | 250 ± 53 | 2.1:1 | 2.1:1 |
| Ac-IEFD-AMC | 1000 ± 99 | 0.15 ± 0.003 | 150 ± 34 | 3.8:1 | 3.3:1 |
| Ac-LEPD-AMC | 87 ± 6.8 | 0.096 ± 0.0055 | 1100 ± 200 | 9.8:1 | 38:1 |

I99F

| | | | | | |
|---|---|---|---|---|---|
| Ac-IEPD-AMC | 510 ± 41 | 0.56 ± 0.03 | 500 ± 80 | | |
| Ac-IETD-AMC | 153 ± 7 | 0.30 ± 0.02 | 1900 ± 320 | 2.0:1 | 1.2:1 |
| Ac-IEFD-AMC | 410 ± 77 | N.D. | | 2.2:1 | 1.25:1 |
| Ac-LEPD-AMC | 200 ± 22 | N.D. | | 2.3:1 | 2.6:1 |

I99R

| | | | | | |
|---|---|---|---|---|---|
| Ac-IEPD-AMC | 330 ± 26 | 0.59 ± 0.02 | 1800 ± 330 | | |
| Ac-IETD-AMC | 150 ± 7 | 0.30 ± 0.03 | 2000 ± 330 | 2.0:1 | 2.2:1 |
| Ac-IEFD-AMC | 505 ± 45 | 0.47 ± 0.01 | 930 ± 190 | 1:1.3 | 1:1.5 |
| Ac-LEPD-AMC | 12 ± 6 | N.D. | | 10:1 | 28:1 |

N218T

| | | | | |
|---|---|---|---|---|
| Ac-IEPD-AMC | 6200 ± 600 | 3.6 ± 0.2 | 580 ± 84 | |
| Ac-IKPD-AMC | 600 ± 4 | 1.1 ± 0.01 | 1800 ± 30 | 10:1 |

N218 Mutations in Combination with I99 or R192 Mutations Dramatically alter Specificity.

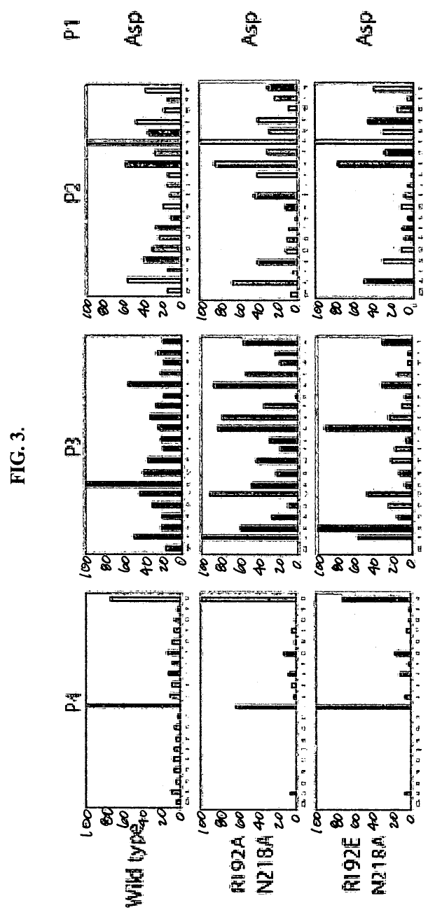

The N218A variant was shown to broaden the P3 specificity of granzyme B, so it was combined with two mutations at the structural determinant Arg192. The R192A single mutation broadens the P3 specificity of rat granzyme B and results in a measured specificity constant of 55 M⁻¹s⁻¹ for Ac-IEPD-AMC, much lower than the nearly wild-type activity of the N218 variant (2200 M⁻¹s⁻¹). In combination, the R192A/N218A variant has no change at P2 and P4, an increased preference for Ala and Ser at P3 (FIG. 3, Table 22), and a slight preference for Glu over Lys. This variant also has low activity similar to the R192A variant (k_{cat}/Kₘ (IEPD)=92 M⁻¹s⁻¹). The R192A variant prefers Glu to Lys by 7 fold whereas the R192A/N218A variant is neutral within error. The PSSCL results indicate a <2 fold preference for Lys over Glu whereas the individual substrates indicate a 2 fold preference for Glu over Lys.

When combined with the R192E mutation, N218A/R192E completely reverses the P3 specificity from acidic to basic (FIG. 3, Table 22). From PSSCL results, the activity of P3-Glu versus Lys has been reversed to favor Lys by nine fold. The kinetic constants demonstrate a significant preference as well. The specificity constants for N218A/R192E for Ac-IEPD-AMC and Ac-IKPD-AMC are 10 M⁻¹s⁻¹ and 73 M⁻¹s⁻¹, respectively (Table 22). This represents a seven fold preference for the basic amino acid, but a 330 fold reduction in activity compared to the wild-type preference for Ac-IEPD-AMC (3300 M⁻¹s⁻¹ versus 10 M⁻¹s⁻¹). The decrease in activity arises from an increase in the Kₘ above the substrate concentration limit of 2 μM. The wild-type activity against the P3-basic substrate Ac-IKPD-AMC is not improved with these mutations (220 M⁻¹s⁻¹ versus 73 M⁻¹s⁻¹). Thus, the double mutation N218A/R192E reverses the P3 specificity of rat granzyme B but the catalysis of P3-Lys substrates does not increase above 73 M⁻¹s⁻¹.

Table 22 shows the kinetic constants for individual tetrapeptide substrates Ac-IEPD-AMC (SEQ ID NO:32), Ac-IKPD-AMC (SEQ ID NO:33) and Ac-IAPD-ACC (SEQ ID NO:42). The preference for acidic amino acids at P3 is totally reversed with the R192E/N218A variant. The highlighted amino acid in each substrates deviate from the preferred wild-type substrate sequence. The presented ratios are of the wild-type preference (P) to the variant's novel preference (X). The novel preference (X) is the bold amino acid of the substrate. Ratios are shown for activity in the PSSCL libraries (PSSCL) and between individual tetrapeptide substrates (Activity).

TABLE 22
Granzyme B WT, N218A/R192A and N218A/R192E mutein specificity.

| | | | | | |
|---|---|---|---|---|---|
| | K_{cat}/Kₘ | K_{cat} | Kₘ | PSSCL | Activity |
| granzyme B | M⁻¹ sec⁻¹ | sec⁻¹ | μM | Ratio (P:X) | Ratio (P:X) |

WT

| | | | | | |
|---|---|---|---|---|---|
| Ac-IEPD-AMC | 3300 ± 500 | 1.2 ± 0.033 | 370 ± 70 | | |
| Ac-IKPD-AMC | 99 ± 5.3 | 0.150 ± 0.01 | 1500 ± 190 | 4.4:1 | 33:1 |
| Ac-IAPD-ACC | 110 ± 4.6 | N.D. | N.D. | 2:1 | 30:1 |

N218A/R192A

| | | | | | |
|---|---|---|---|---|---|
| Ac-IEPD-AMC | 92 ± 11 | 0.16 ± 0.01 | 1700 ± 730 | — | — |
| Ac-IKPD-AMC | 55 ± 2.8 | N.D. | >2000 | 1:1.5 | 1.7:1 |
| Ac-IAPD-ACC | 29 ± 1.3 | N.D. | | 1:1.9 | 3.2:1 |

N218A/R192E

| | | | | | |
|---|---|---|---|---|---|
| Ac-IEPD-AMC | 10 ± 0.35 | N.D. | >2000 | — | — |
| Ac-IKPD-AMC | 73 ± 3.4 | N.D. | >2000 | 1:14 | 1:7 |
| Ac-IAPD-ACC | 15 ± 1.5 | N.D. | | 1:9 | 1:1.5 |

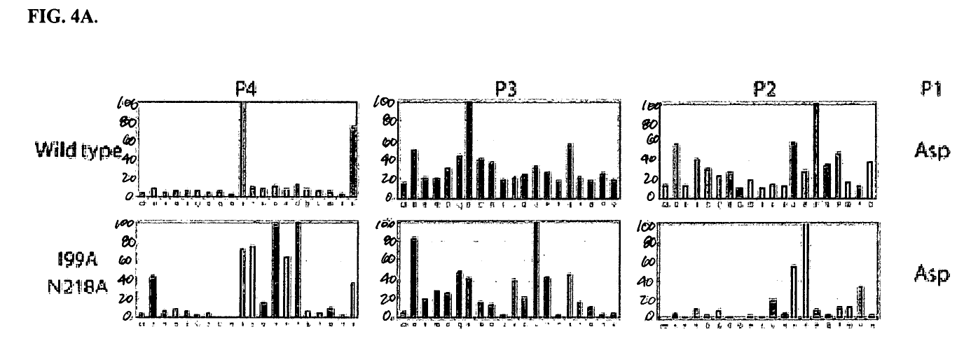

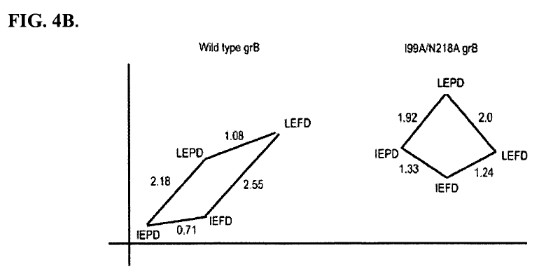

The double mutation N218A/I99A had a dramatic effect on extended specificity altering the P2 through P4 specificity by PSSCL to the sequence Pro-Ala-Phe-Asp (FIG. 4, Table 23). Like the I99A and I99R variants, the P2 specificity is narrowed to prefer large hydrophobic amino acids, but in addition the P3 and P4 specificity have been broadened to include most aliphatic and β branched amino acids. The activity of the N218A/I99A variant is comparable to the activity of the I99A variant against the Ac-IEFD-AMC substrate (270 M⁻¹s⁻¹ versus 290 M⁻¹s⁻¹, respectively), though the tetrapeptide substrate with the highest specificity constant is Ac-IEFD-AMC (290 M⁻¹s⁻¹) rather than Ac-LEFD-AMC (37 M⁻¹s⁻¹). This effect is due to an increase in the binding constant (890 μM versus >2000 μM), suggesting that some cooperativity in substrate preference is masked by the combinatorial nature of the libraries.

Table 23 shows the kinetic constants for the tetrapeptide substrates Ac-IEPD-AMC (SEQ ID NO:32), Ac-IEFD-AMC (SEQ ID NO:35), Ac-LEPD-AMC (SEQ ID NO:37), and Ac-LEFD-AMC (SEQ ID NO:36) and the calculated change in free energy compared to the wild-type sequence IEPD.

TABLE 23
Granzyme B wild-type and I99A/N218A mutein specificity

| | | | | |
|---|---|---|---|---|
| | | | | ΔΔG_{T}^{‡} |
| | k_{cat}/Kₘ | k_{cat} | Kₘ | Kcal |
| granzyme B | M⁻¹ sec⁻¹ | sec⁻¹ | μM | mol⁻¹ |

WT

| | | | | |
|---|---|---|---|---|
| Ac-IEPD-AMC | 3300 ± 500 | 1.2 ± 0.033 | 370 ± 70 | 0 |
| Ac-IEFD- | 1000 ± 99 | 0.154 ± 0.003 | 150 ± 34 | +0.71 |
| AMC | | | | |
| Ac-LEPD- | 88 ± 6.8 | 0.096 ± 0.0006 | 1100 ± 200 | +2.18 |
| AMC | | | | |
| Ac-LEFD- | 14 ± 0.86 | .0087 ± 0.0003 | 600 ± 260 | +3.26 |
| AMC | | | | |

I99A/N218A

| | | | | |
|---|---|---|---|---|
| Ac-IEPD-AMC | 32 ± 1.9 | 0.087 ± 0.0008 | 2700 ± 590 | 0 |
| Ac-IEFD- | 290 ± 24 | 0.26 ± .002 | 890 ± 350 | −1.33 |
| AMC | | | | |
| Ac-LEPD- | 1.3 ± 0.11 | N.D. | >2000 | +1.92 |
| AMC | | | | |
| Ac-LEFD- | 37 ± 9.0 | 0.047 ± 0.0005 | 1300 ± 168 | −0.09 |
| AMC | | | | |

Specificity Determinants Role in the Transition State

To understand the extent of P2 and P4 transition state stabilization in rat granzyme B, a series of gradually substituted substrates (Ac-IEPD-AMC, Ac-LEPD-AMC, Ac-IEFD-AMC, and Ac-LEFD-AMC) were kinetically characterized for wild-type and N218A/I99A granzyme B (Table 24). The extent of cooperativity between the extended subsites is determined by the difference between the transition-state stabilization energy (50) calculated using the difference in specificity, k_{cat}/Kₘ, between a substituted substrate and the wild-type IEPD substrate:
ΔΔG_{T}⁵⁵⁵=−R_{T} ln [(k_{cat}/Kₘ)_{IEPD}/k_{cat}/Kₘ)_{modified}] (1)

The difference in free energy will be positive if the wild-type substrate, IEPD, is a better substrate than the modified substrate. There is cooperativity when the ΔΔG_{T}^{‡}for a single amino acid modification does not equal the ΔΔG_{T}^{‡}for the same modification made in the context of an additional modification. Therefore, when graphed a series of substrates with no cooperativity forms a rectangle while a cooperative series does not.

In wild-type granzyme B, the ΔΔG_{T}^{‡}for P2-Pro versus P2-Phe is +0.71 and +1.1 kcal/mol when P4 is Ile or Leu, respectively (Table 24). The same result is observed for the P4 amino acid change for Ile to Leu ΔΔG_{T}^{‡}+2.18 and 2.55 kcal/mol). This highlights the wild-type granzyme B requirement of an extended substrate for efficient hydrolysis. The identity of the P4 amino acid alone contributes approximately 1.5 kcal/mol to the stabilization of the transition state. When a nonpreferred amino acid or no amino acid at all is present in the substrate, the activity is reduced significantly. The N218A/I99A variant then reverses this result. The ΔΔG_{T}^{‡}for P2-Pro versus P2-Phe is −1.33 kcal/mol when the P4 amino acid is Ile, and −2 kcal/mol when the P4 amino acid is Leu. Cooperativity is also observed when the P4 amino acid is changed from Ile to Leu (ΔΔG_{T}^{‡}+1.92 and +1.24 kcal/mol for P2-Pro and P2-Phe, respectively.) The N218A/I99A variant is exhibiting a cooperative effect because the amount of binding energy contributed to the transition state stabilization from an ideal or non-ideal P2 amino acid is dependent on the identity of the P4 amino acid.

Discussion

The granzyme B-like subfamily of serine proteases play an important role in granule-mediated immune responses. Clustered on chromosome 14 of human, rat and mouse species, they share active site architecture distinct from trypsin-like proteases. Computational analysis indicates that the four loop regions comprising the P1 through P4 pockets in combination with the catalytic triad, the calcium and the sodium binding sites represent sufficient diversity to replicate the phylogenic tree of full-length serine proteases. Thus the primary differentiators of the various sub-families of the trypsin fold proteases are located in these areas. These amino acids in the granzyme B-like subfamily are composed of invariant amino acids that make them distinct from the coagulation and digestive proteases, such as Tyr215, and variable amino acids that give the family its diversity, such as 99, 192, and 218.

The most dramatic alteration to granzyme B extended specificity occurred at the P2 position, first when Ile99 was mutated to Arg and Ala and then in combination with N218A. The Ile99Ala mutation likely acts to increase the size and hydrophobicity of the S2 pocket, decreasing in turn the amount of binding energy a non-optimal amino acid contributes towards catalytic activity. I99A granzyme B hydrolyzes the substrate Ac-IEFD-AMC with a higher binding constant than the wild-type enzyme, while I99R has a lower Kₘ. Therefore, the size increase of the S2 pocket in I99A may not orient the substrate for efficient catalysis as effectively as the long aliphatic side chain of Arg99. Alanine is not found at amino acid 99 of any granzyme member or any trypsin fold serine protease, but other small amino acids such as Val and Gly are present. Arg is unique at position 99, found only in granzymes C and A. Both the human and mouse homologs of granzyme A have hydrophobic preferences at P2. Also of note is the increased P4-Leu preference in the I99F variant. This subtle difference between the rat (99-Ile), mouse (99-Phe), and human (99-Phe) homologues of granzyme B significantly alters small molecule inhibitor binding between species.

Previous experiments confirm that amino acid 99 in coagulation proteases are responsible for differences in the substrate selectivity and inhibitor binding of activated protein C, thrombin and Factor Xa. The I99A, I99R and I99F variants highlight the universal role of position 99 as a determinant of P2 specificity, and the differences in active site architecture such as the length and composition of the 60's loop (a determinant of thrombin P2-Pro specificity) and that may also determine specificity.

Kinetic analysis demonstrated that amino acid 218 plays an important determining role in P3 specificity despite experiments implicating amino acid 192 as the main determinant. Arg192 is a determinant of extended specificity in granzyme B, activated protein C, thrombin, and Factor Xa. In granzyme B, P3 specificity is determined by both amino acids 218 and 192. Arg192 provides a strong electrostatic repulsion against Arg and Lys substrate side chains, while the polar Asn218 acts as a selector to exclude large hydrophobic and β branched amino acids. Asn218 serves as a steric determinant through the exclusion of large side chains from S3, and as a hydrogen bond donor for the wild-type Glu/Gln/Met preference. The removal of the hydrogen bonding amino group of Asn218 and its truncation lowered the specificity for charged substrates and increased the activity of small amino acids such as Ser and Ala. They likely bind to the surface by forming favorable non- specific van der Waals interactions. Yet, the role of Asn218 is not limited to P3 interactions. Two results point to a role for Asn218 in orienting the substrate. First, the Y174A mutation did not alter the P4 specificity, yet the addition of N218A to I99A had a dramatic effect. The truncation of 218 increases the degrees of freedom of the substrate backbone allowing additional hydrophobic amino acids to be accommodated by P4 while still retaining efficient hydrolytic activity. Second, the N218T variant was twice as efficient at cleaving the Ac-IEPD-AMC substrate as wild-type entirely due to an increase in the k_{cat} of the protease. The mutations at this position also decouple the interdependence between the P2 and P4 positions.

Cooperativity and interdependence between the extended substrate sites has been described in the serine protease subtilisin. A favorable substrate amino acid at the P1 or P4 positions reduces the effect of a disfavored amino acid at another site. An effective catalytic ceiling exists where the maximum amount of ground state substrate binding energy is converted to transition state stabilization and catalysis when P4 and P1 are ideal amino acids. Extensive studies with the protease subtilisin indicate that hydrophobic, steric and structural effects all contribute to efficient catalysis of substrates by modulating the formation of the Michaelis complex. Mutations to subtilisin successfully alter the specificity and retain efficient catalytic rates. This is not the same for granzyme B, reflecting the fundamental difference between the their three-dimensional structures. The P4 site of WT granzyme B is energetically unlinked to the P2 site. The change in rate when the P2 substrate amino acid is varied is nearly independent of the identity of the P4 amino acid. Mutation of extended specificity determinants changed the relative importance and cooperative effects of extended specificity as observed for alanine substituted substrates against WT and N218A/I99A granzyme B. Perhaps subtilisin is a more flexible protease capable of adapting to mutagenesis that alters extended specificity without the equivalent reduction in activity, a situation where ground state binding is tightly coupled to access to the transition state.

The results of the systematic mutagenesis described here demonstrate that amino acids in direct contact with the extended substrate amino acids by side chain to side chain interactions contribute to the extended specificity of the enzyme. Mutation of the structural determinants changed the specificity and both the binding constant, Kₘ, and the catalytic rate, k_{cat}. Importantly, the hydrolysis by the variant proteases of their most preferred substrate was within 2 fold of the activity of the wild-type protease against the same substrate. The variant proteases retain hydrolytic activity, binding constants and catalytic rates on the order of a nonideal wild-type substrate. The alterations to specificity arise from the exclusion of non-ideal substrates rather than increased affinity of ideal substrates. Thus by examining the amino acids that line the active site cleft of a granzyme B-like protease, logical predictions of extended specificity can be anticipated. Position 99 has been well defined as a determinant of P2 specificity, and the combination of 192 and 218 defines P3. By applying the results of this study to the remaining granzymes, their roles can be defined as unique rather than redundant. Each member has distinct characteristics at extended positions that suggest disparate roles in the apoptotic cascade.

Although the invention has been described with respect to specific methods of making and using granzyme B enzymes capable of cleaving target VEGFR polypeptide sequences, it will be apparent that various changes and modifications may be made without departing from the invention.

### Example 17

### Muteins Consisting of up to Four Mutations with Increased Selectivity Towards VEGFR Stalk Region Sequence, LVED

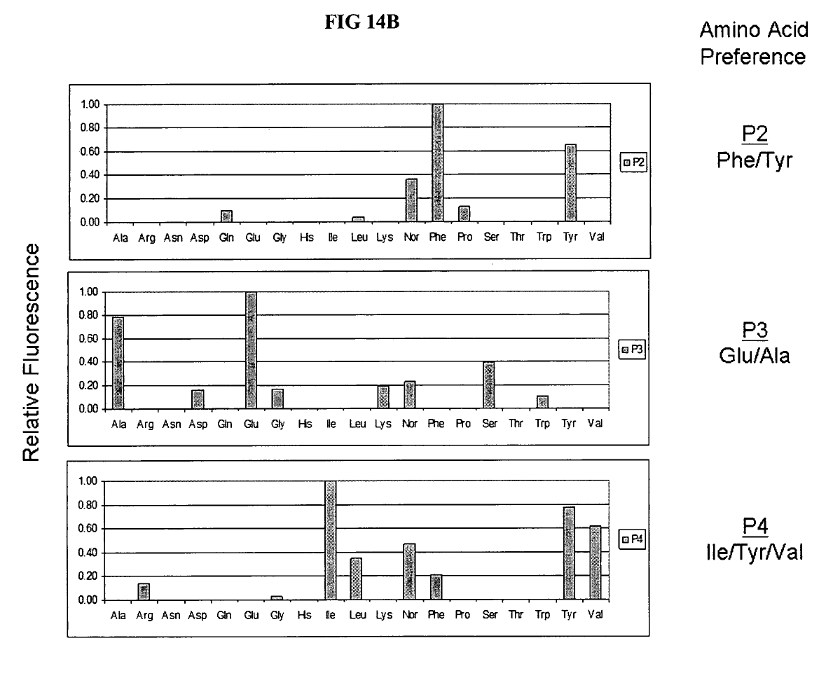

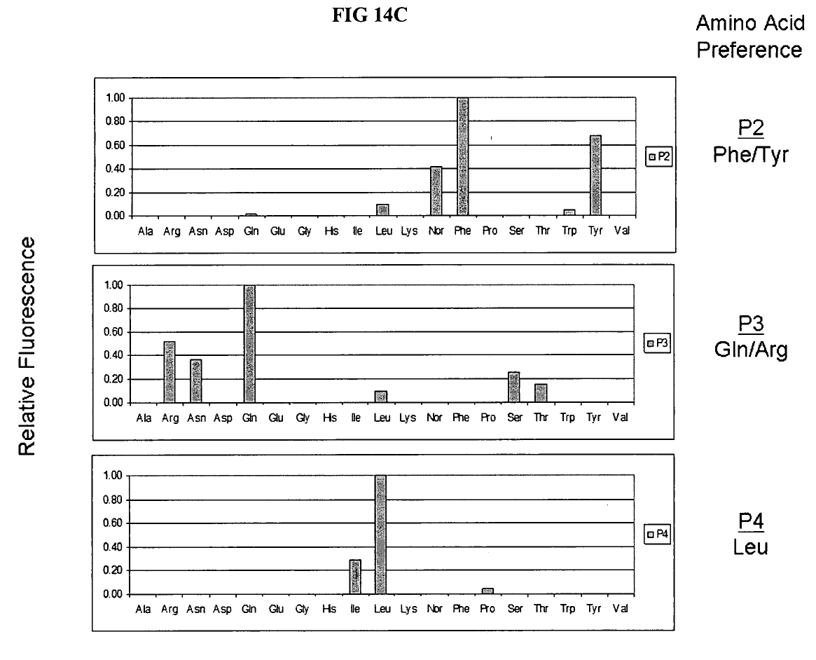

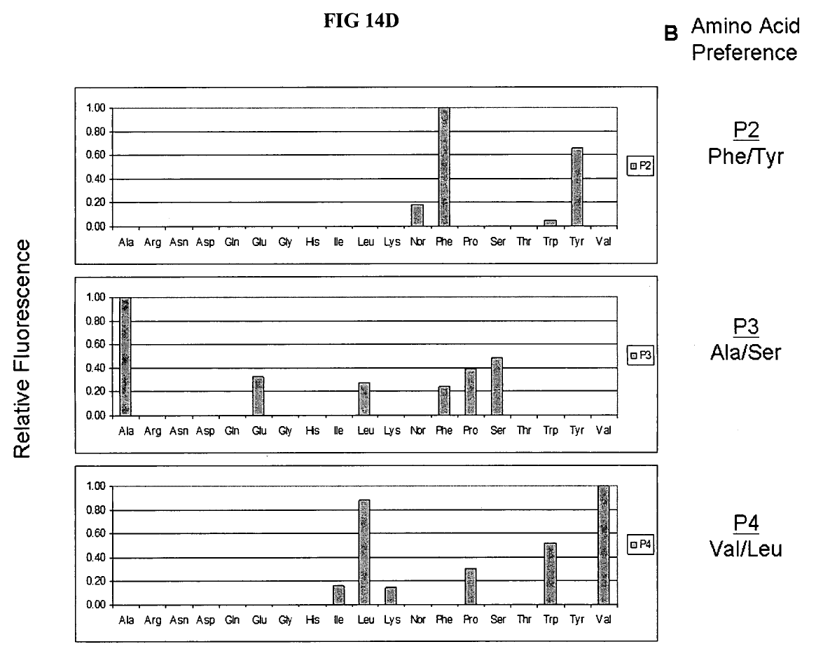

Multiple muteins were identified by PSSCL profiling with increased selectivity towards the LVED target cleavage sequence. They were grouped into sub-classes based on which subsite profile was most affected by the mutations: P4, P3 or P2. In addition to the muteins discussed in Example 16, mutations were found at four additional sites to have a role in the protease selectivity. Mutations of Ile99 to Asp, Tyr and Ala increased the P2 selectivity towards polar and large hydrophobic amino acids. Mutations of Asn218 (Gln, Asp), Tyr151 (Ala), and Arg192 (Tyr, Met) all distinctly alter the P3 specificity of granzyme B away from the wild-type Glu/Gln/Met preference and towards small polar, acidic, and large polar containing substrates, respectively (FIG. 14A). At the P4 position of the substrate, mutations at Lys97 (Glu, Tyr), Leu171 (Glu), Tyr174 (Glu, Gln), Tyr215 (Asn) increase the selectivity of granzyme B towards additional hydrophobic amino acids such as Trp, Phe, Tyr and Leu (FIG. 14B).

Results. By grouping mutations identified individually to narrow the protease selectivity at P2, P3 and P4, variants were made that had greater that had selectivity towards Leu residues at the P4 position and altered P2 and P3 specificity. Two variants, R192Y/I99A/N218A (CB111) and K97E/I99A/N218A/Y174E (CB121) strongly preferred Leu at P4 to other amino acids. CB111 preferred Leu at least three fold over Ile and to the near exclusion of any other amino acid (FIG. 14C & D). These characteristics in the PSSCL demonstrate the efficacy of mutations from Table 12 on altering the selectivity of the granzyme B protease towards the desired LVED sequence.

### Example 18

### Methods of Cloning and Characterizing Engineered MT-SP1 Protease with Altered Substrate Specificity Based on Well Understood Starting Scaffolds

The serine protease MT-SP1 has been chosen as scaffold protease for mutagenesis towards specific proteolysis of the VEGF and VEGFR in part because it has been well characterized with biochemical and structural techniques [Harris, Recent Results Cancer Res. 1998; 152:341-52].

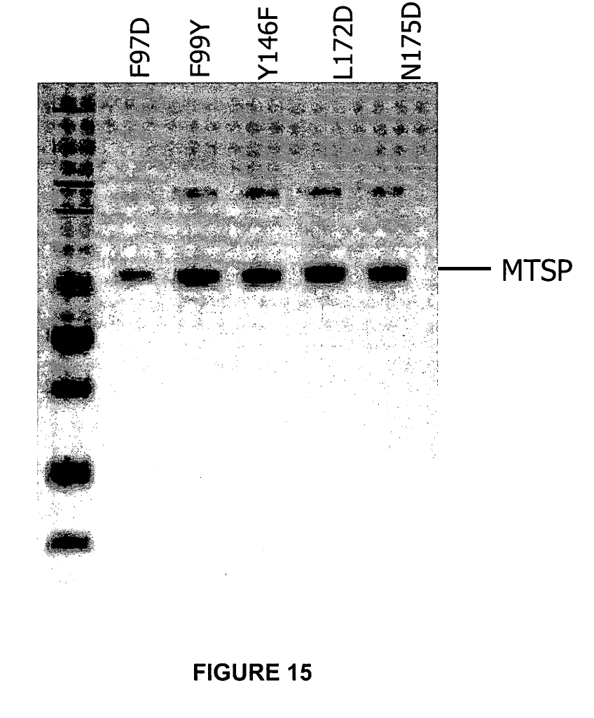

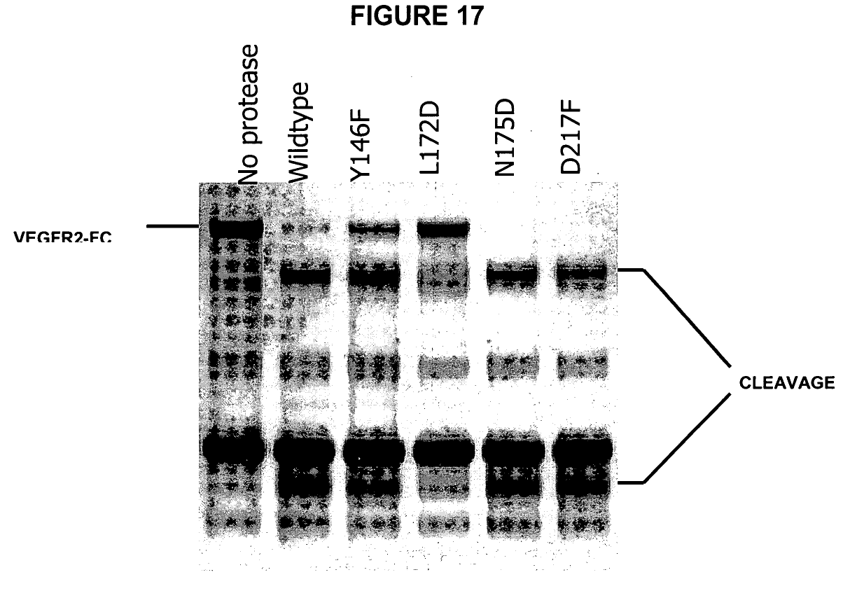

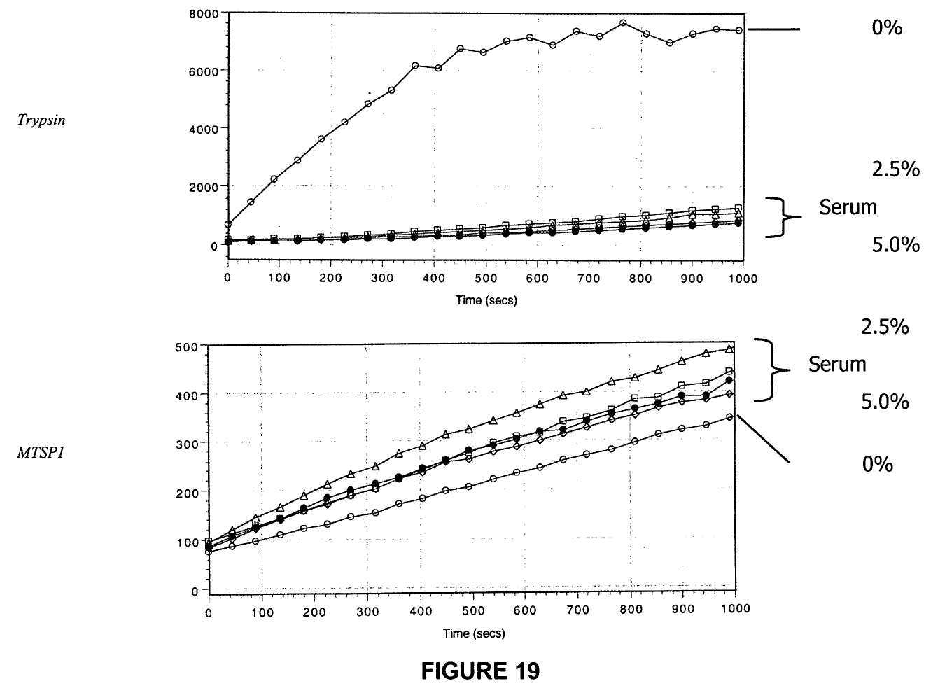

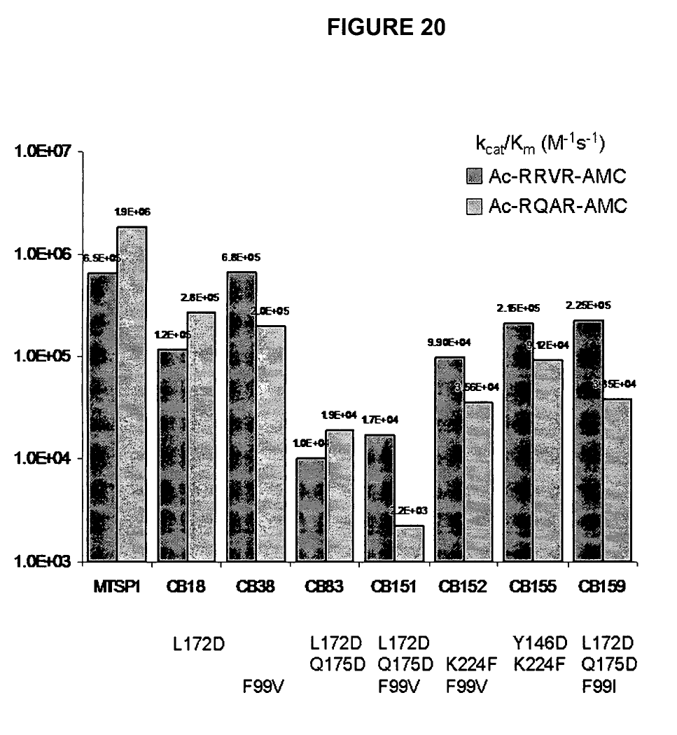

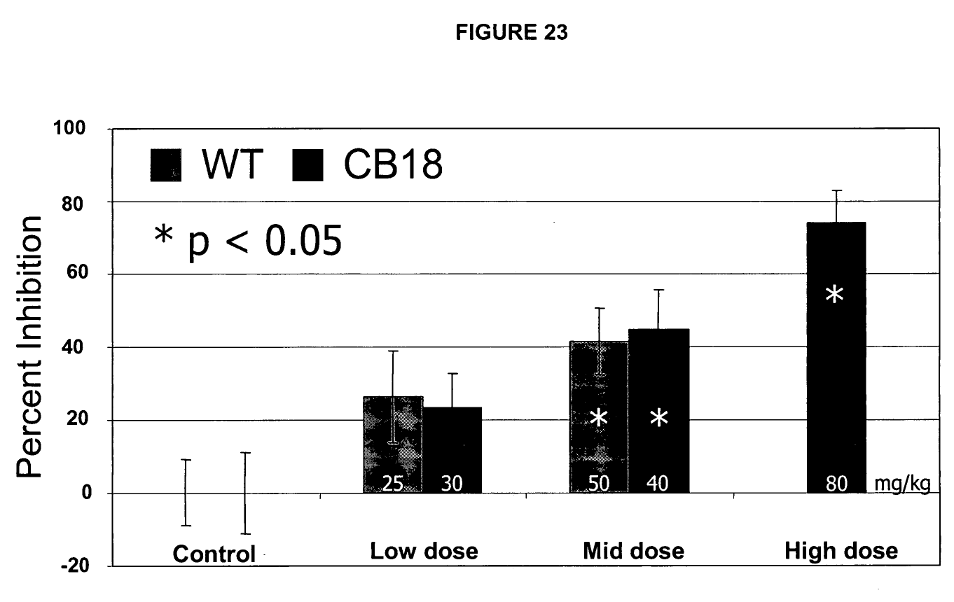

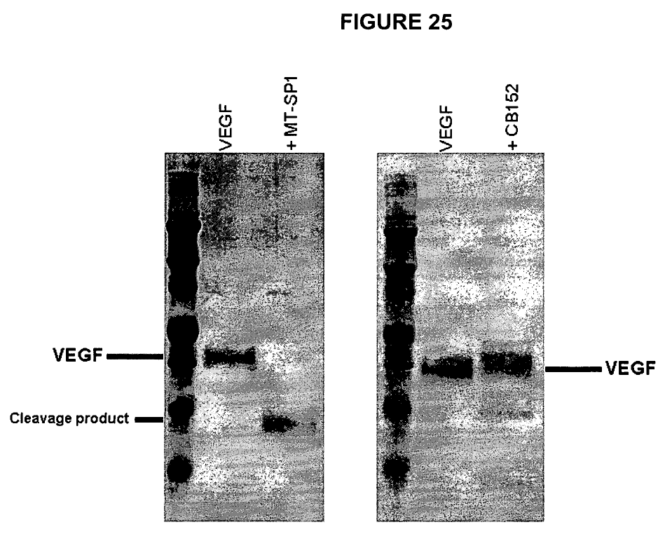

MT-SP1 is a membrane bound serine protease with multiple extracellular protein-protein interaction domains. The protease domain alone has been profiled using the totally diverse and P1-Lys PSSCL (FIG. 19A-19C) revealing an extended specificity of (basic)-(non-basic)-Ser-Arg or (non-basic)-(basic)-Ser-Arg/Lys. The X-ray crystallographic structure of MT-SP1 reveals components proposed to regulate activity and a nine amino acid insertion in the 60's loop that may determine P2 specificity Variants of MT-SP1 have been created and characterized. Various protease muteins have been expressed and purified, as described below. Initial activity to verify activity and specificity have been performed, and sample results are provided in FIGS. 15-25.

### Example 19

### Expression and Purification of MT-SP1

A mutated MT-SP1 polypeptide (“mutein”) may contain a single mutation per polypeptide, or may contain two or more mutated residues in any combination provided as illustrated in Tables 17 and 18.

Wild-type and mutant MT-SP1 are cloned into the pQE bacterial expression vector (Qiagen) containing an N-terminal 6 histidine tag, prodomain, and protease domain and the resulting constructs transformed into BL21 E. coli cells. Cells are grown in 100 mL cultures to an OD of 0.6 and expression of the protease in inclusion bodies is induced by adding IPTG to a final concentration of 1 mM. After 4-6 hours, the bacteria are pelleted by centrifugation and the pellet resuspended in 50 mM Tris pH 8, 500 mM KCl, and 10% glycerol (buffer A). Cells are lysed by sonication and pelleted by centrifugation at 6000×g. Pellets are resuspended in 50 mM Tris pH 8, 6 M urea, 100 mM NaCl and 1% 2-mercaptoethanol (buffer B). Membrane and organelles are pelleted by centrifugation at 10,000×g and the supernatant is passed over a nickel NTA column (Qiagen). The column is washed with 50 mM Tris pH8, 6 M urea, 100 mM NaCl, 20 mM imidazole, 1% 2-mercaptoethanoland 0.01% Tween 20 (buffer D). The column is washed again with buffer D without Tween 20. The protease is then eluted from the column with 50 mM Tris pH 8, 6 M urea, 100 mM NaCl, 1% 2-mercaptoethanol and 250 mM imidazole (buffer E). The protease is then concentrated to a volume of ˜1 mL and then dialyzed at 4° C. overnight in 1 L of 50 mM Tris pH8, 3 M urea, 100 mM NaCl, 1% 2-mercaptoethanol, and 10% glycerol. Finally, the protease is dialyzed into 50 mM Tris pH 8, 100 mM NaCl, and 10% glycerol at 4° C. overnight. During the last dialysis step, the protease becomes autoactivated by self-cleavage resulting in the removal of the 6 histidine tag and prodomain.

Result. Multimilligram quantities are obtained using bacterial expression system. The protease is produced in inclusion bodies and is purified by a one-column purification procedure and then re-folded through successive dialysis steps (FIG. 15). Once refolded, the protease activates itself by cleavage at the juncture between the prodomain and the protease domain at the sequence RQAR/VVGG.

### Example 20

### Determination of the Extended Specificity of MT-SP1 Variants by PSSCL

The P1-Arg fixed PSSCL library is resuspended in DMSO and arrayed in opaque black 96-well plates at a concentration of 5-10 nanomoles per well. Variant proteases are diluted into 50 mM Tris pH 8, 50 mM NaCl, and 0.01% Tween 20 (MT-SP1 activation buffer) at a concentration of 5 nM to 5 μM. One hundred microliters of the protease solution is added to each well and fluorescence of the ACC leaving group is measured by excitation at 380 nm and emission at 460 nm using a Spectramax fluorescent plate reader (Molecular Devices). The specificity of variant proteases at each of the P4-P2 extended subsites is determined by the fluorescence of each of the arrayed amino acids in the P4-P2 PSSC libraries.

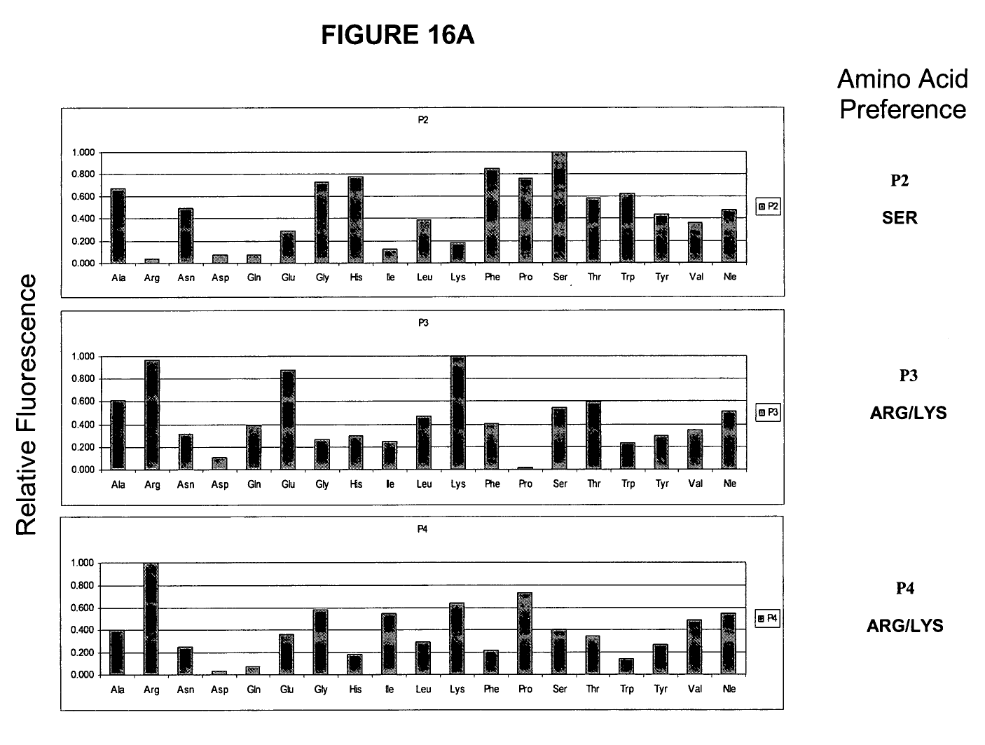

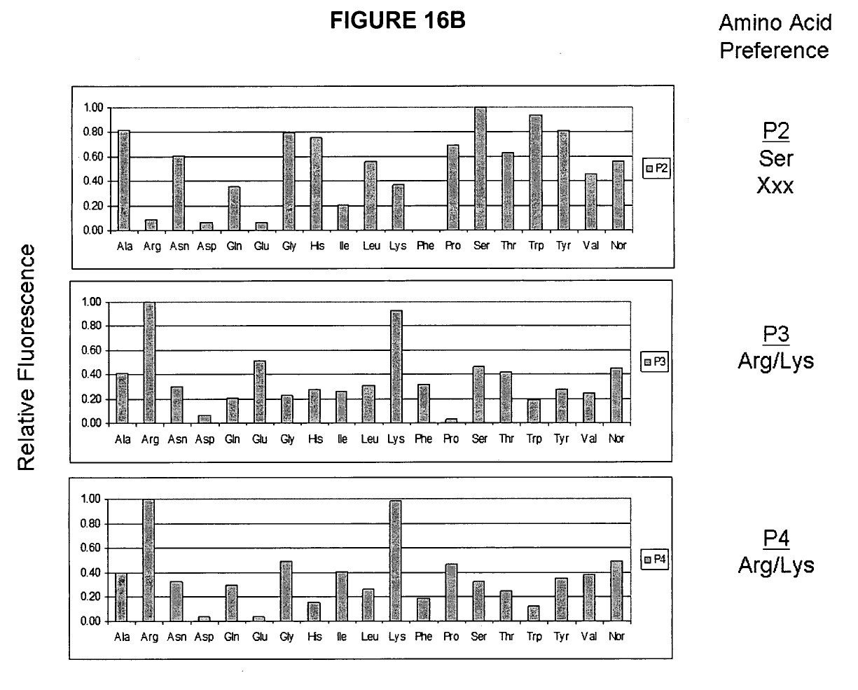

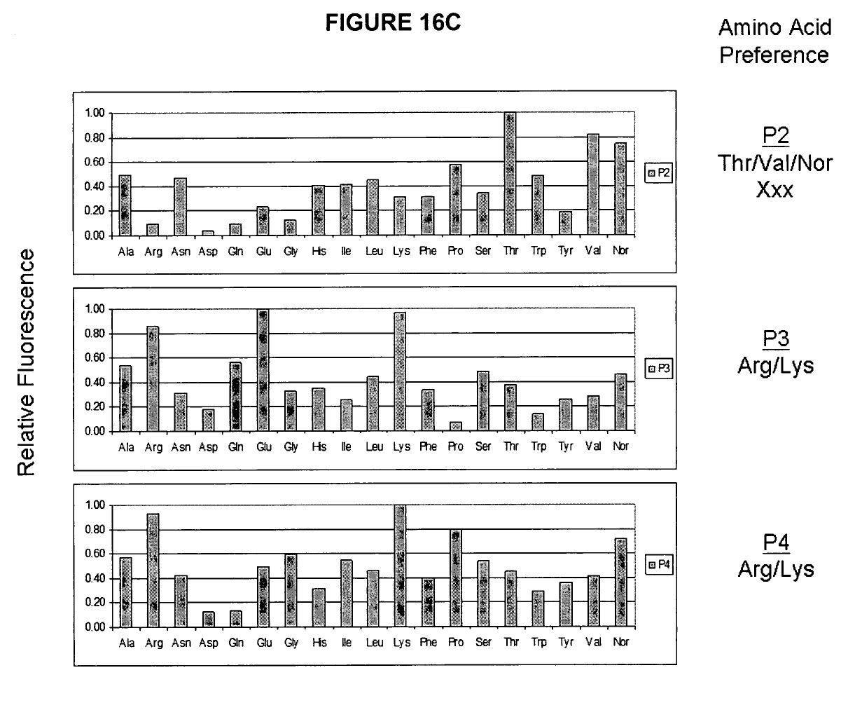

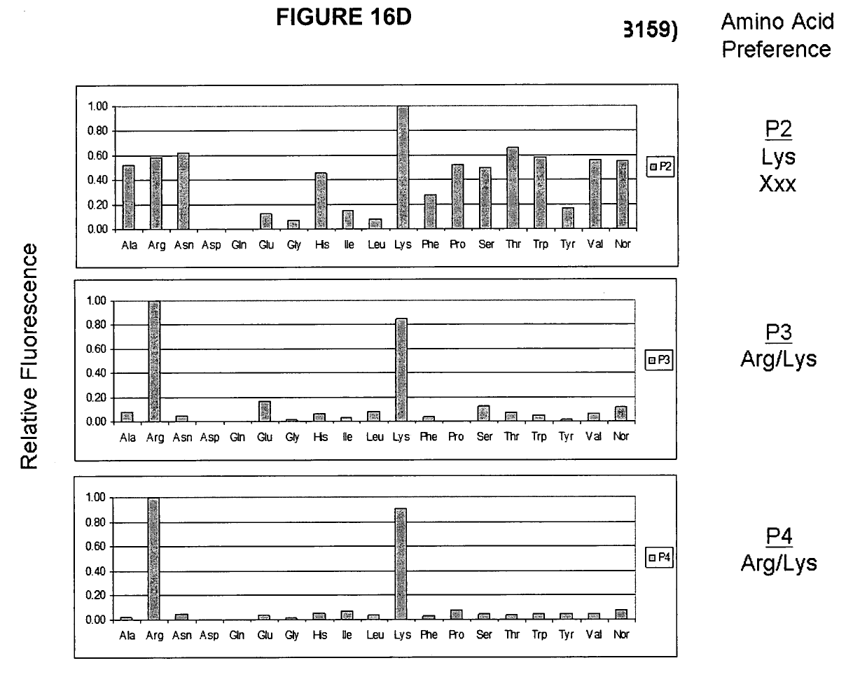

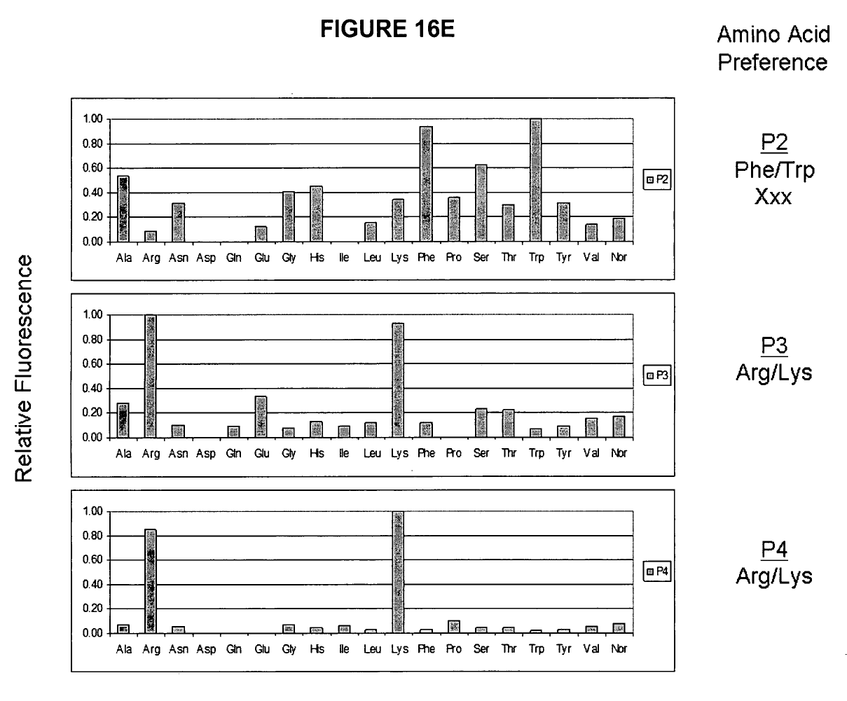

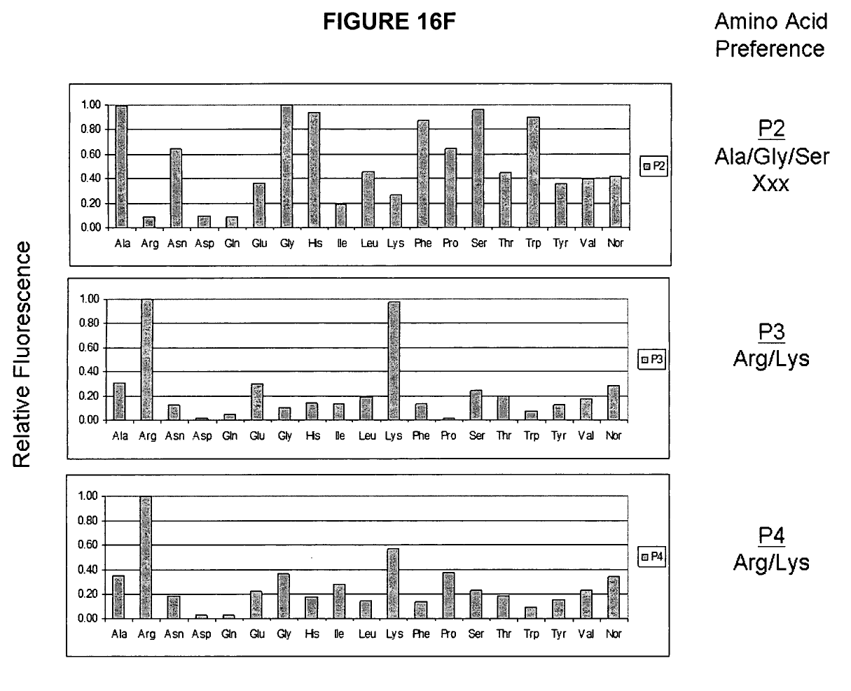

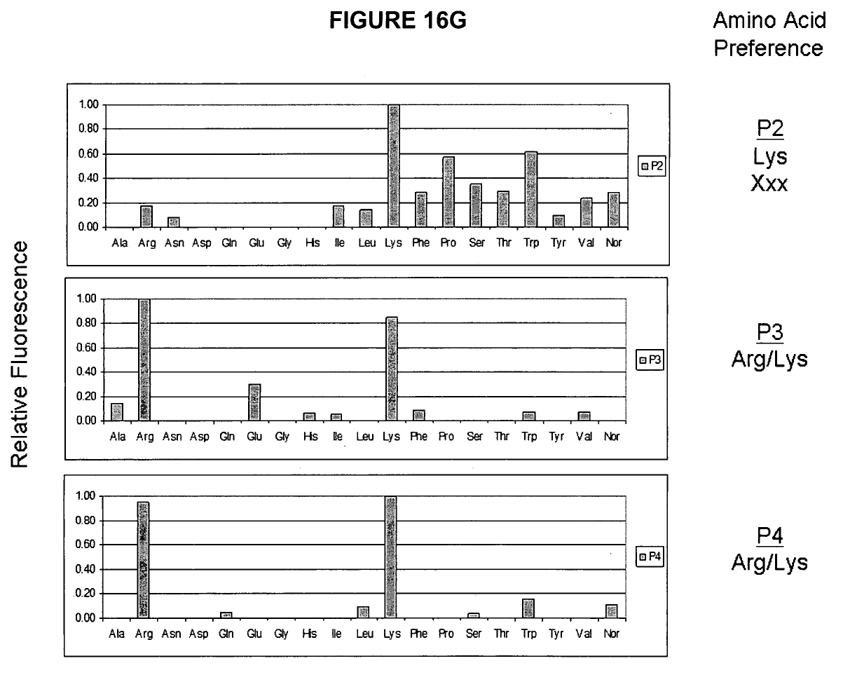

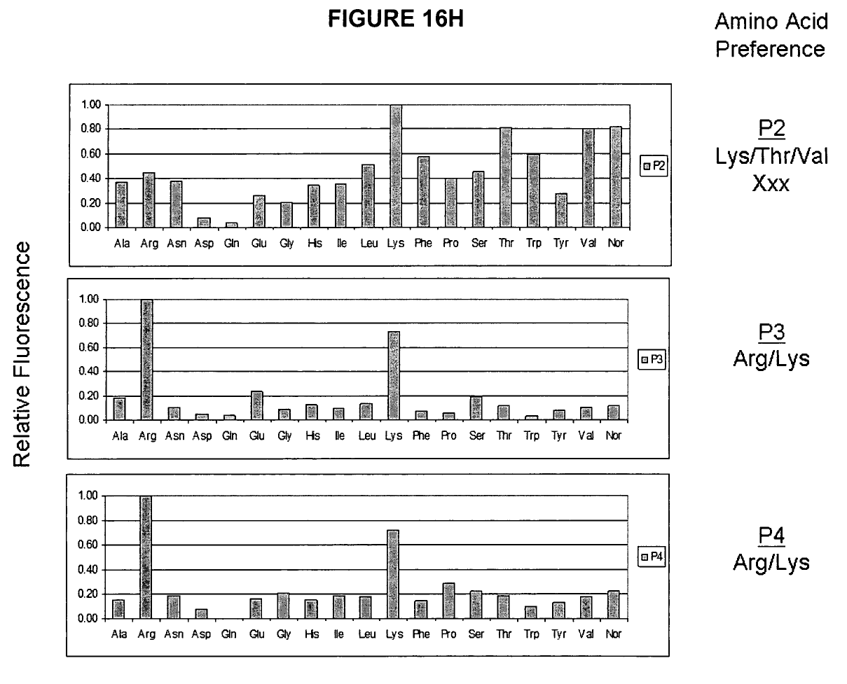

Result. Screening by PSSCL confirms that wild-type MT-SP1 has a preference for basic (Arg, Lys) at the P4 and P3 positions, in agreement with published data by Takeuchi et al., J. Biol. Chem., Vol. 275, Issue 34, 26333-26342, Aug. 25, 2000. However, the PSSCL profile also reveals that its specificity is somewhat broad, such that a variety of amino acids will be accepted in the P4 and P3 positions in addition to Arg or Lys (FIG. 16A). A number of mutants were generated (see above) to narrow the substrate specificity and to direct it towards potential cleavage sites identified in the VEGF receptor (see below). One mutant, L172D (CB18), shows a very narrow specificity profile, such that Arg or Lys is strongly preferred over any other amino acid in the P4 and P3 positions (FIG. 16B). A potential cleavage sequence has been identified in VEGFR2 (RRVR) that closely matches the specificity profile for L172D (RRXR). Variants of MT-SP1 have been profiled with the P1-Arg PSSCL (for specific variants see Tables 17 and 18). All variants show an increase in selectivity at one or more substrate sequence positions. Representative profiles are shown in FIGS. 16A through H.

### Example 21

### Selection of MT-SP1 Variants Capable of Peptide Sequence Specific Target Cleavage Using Protease Phage Display

The phagemid is constructed such that it (i) carries all the genes necessary for M13 phage morphogenesis; (ii) it carries a packaging signal which interacts with the phage origin of replication to initiate production of single-stranded DNA; (iii) it carries a disrupted phage origin of replication; and (iv) it carries an ampicillin resistance gene.

The combination of an inefficient phage origin of replication and an intact plasmid origin of replication favors propagation of the vector in the host bacterium as a plasmid (as RF, replicating form, DNA) rather than as a phage. It can therefore be maintained without killing the host. Furthermore, possession of a plasmid origin means that it can replicate independent of the efficient phage-like propagation of the phagemid. By virtue of the ampicillin resistance gene, the vector can be amplified, which in turn increases packaging of phagemid DNA into phage particles.

Fusion of the MT-SP1 variant gene to either the gene 3 or gene 8 M13 coat proteins can be constructed using standard cloning methods. (Sidhu, Methods in Enzymology, 2000, V328, p333). A combinatorial library of variants within the gene encoding MT-SP1 is then displayed on the surface of M13 as a fusion to the p3 or p8 M13 coat proteins and panned against an immobilized, aldehyde-containing peptide corresponding to the target cleavage of interest. The aldehyde moiety will inhibit the ability of the protease to cleave the scissile bond of the protease, however, this moiety does not interfere with protease recognition of the peptide. Variant protease-displayed phage with specificity for the immobilized target peptide will bind to target peptide coated plates, whereas non-specific phage will be washed away. Through consecutive rounds of panning, proteases with enhanced specificity towards the target sequence can be isolated. The target sequence can then be synthesized without the aldehyde and isolated phage can be tested for specific hydrolysis of the peptide.

### Example 22

### Identification of MT-SP1 Mutein Cleavage in the Stalk Region of VEGFR2

The polypeptide sequence of VEGF receptor 2 (VEGF-R2/KDR), showing the respective sequences of the extracellular (SEQ ID NO:38) and intracellular (SEQ ID NO:39) domains, is provided in Table 12. Sequences that closely match the P4-P1native substrate specificity of MT-SP1 are shown in bold. Two sequences match the recognition profile of both L172D and wild-type MT-SP1: the boxed sequence RVRK and the double underlined sequence RRVR.

TABLE 24
VEGFR2/KDR Substrate Specificity of Targeted MT-SP1 Proteases

| | | |
|---|---|---|
| Extra-cellular | KQSKVLLAVALWLCVETRAASVGLPSVSLDLPRLSIQKDILTIKANTTLQITCRGQRDLD | (SEQ ID NO:38) |
| | WLWPNNQSGSEQRVEVTECSDGLFCKTLTIPKVIGNDTGAYKCFYRETDLASVIYVYVQD | |
| | YRSPFIASVSDQHGVVYITENKNKTVVIFCLGSISNLNVSLCARYPEKRFVPDGNRISWD | |
| | SKKGFTIPSYMISYAGMVFCEAKINDESYQSIMYIVVVVGYRIYDVVLSPSHGIELSVGE | |
| | KLVLNCTARTELNVGIDFNWEYPSSKHQHKKLVNRDLKTQSGSEMKKFLSTLTIDGVTRS | |
| | DQGLYTCAASSGLMTKKNSTFVRVHEKPFVAFGSGMESLVEATVGERVRIPAKYLGYPPP | |
| | EIKWYKNGIPLESNHTIKAGHVLTIMEVSERDTGNYTVILTNPISKEKQSHVVSLVVYVP | |
| | PQIGEKSLISPVDSYQYGTTQTLTCTVYAIPPPHHIHWYWQLEEECANEPSQAVSVTNPY | |
| | PCEEWRSVEDFQGGNKIEVNKNQFALIEGKNKTVSTLVIQAANVSALYKCEAVNKVGRGE | |
| | RVISFHVTRGPEITLQPDMQPTEQESVSLWCTADRSTFENLTWYKLGPQPLPIHVGELPT | |
| | PVCKNLDTLWKLNATMFSNSTNDILIMELKNASLQDQGDYVCLAQDRKTKKRHCVVRQLT | |
| | VLERVAPTITGNLENQTTSIGESIEVSCTASGNPPPQIMWFKDNETLVEDSGIVLKDGNR | |
| | NLTIRRVRKEDEGLYTCQACSVLGCAKVEAFFIIEGAQEKTNLE | |
| Intra-cellular | IIILVGTAVIANFFWLLLVIILRTVKRANGGELKTGYLSIVMDPDELPLDEHCERLPYDA | (SEQ ID NO:39) |
| | SKWEFPRDRLKLGKFLGRGAFGQVIEADAFGIDKTATCRTVAVKNLKEGATHSEHRALMS | |
| | ELKILIHIGHHLNVVNLLGACTKPGGPLMVIVEFCKFGNLSTYLRSKRNEFVPYKTKGAR | |
| | FRQGKDYVGAIPVDLKRRLDSITSSQSSASSGFVEEKSLSDVEEEEAFEDLYKDFLTLEH | |
| | LICYSFQVAKGMEFLASRKCIHRDLAARNILLSEKNVVKICDFGLARDIYKDPDYVRKGD | |
| | ARLPLKWMAPETIFDRVYTIQSDVWSFGVLLWEIFSLGASPYPGVKIDEEFCRRLKEGTR | |
| | MRAPDYTTPEMYQTMLDCWHGEPSQRPTFSELVEHLGNLLQANAQQDGKDYIVLPISETL | |
| | SMEEDSGLSLPTSPVSCMEEEEVCDPKFHYDNTAGISQYLQNSKRKSRPVSVKTFEDIPL | |
| | EEPEVKVIPDDNQTDSGMVLASEELKTLEDRTKLSPSFGGMVPSKSRESVASEGSNQTSG | |
| | YQSGYHSDDTDTTVYSSEEAELLKLIEIGVQTGSTAQILQPDSGTTLSSPPV | |

Purified extracellular domain of VEGF-R2 (Flk1) fused to the Fc domain of mouse IgG (2.5 μg) was resuspended with 1 μM MT-SP1 and variant proteases in 17.1 uL of MT-SP1 activation buffer. The reaction was incubated at 37° C. for 2 hours, deglycosylated with PNGaseF, and separated by SDS-PAGE electrophoresis. Full length Flk1-Fc and cleavage products were identified by staining with Coomassie brilliant blue and the N-termini sequenced by the Edman protocol. Purified VEGFR2-Fc is cleaved by wild-type and mutant MT-SP1 at the sequence RRVR/KEDE in the extracellular stalk region of the receptor. Thus, the present invention provides proteases that can cleave the VEGFR in the stalk region, and in one embodiment of the invention, such proteases are administered to a patient in need of treatment for cancer, macular degeneration, or another disease in which angiogenesis plays a causative or contributive role.

### Example 23

### Assaying Cleavage of Purified VEGF Receptor

Purified extracellular domain of VEGF-R2 fused to the Fc domain of mouse IgG (3-10 μg) is resuspended in MT-SP1 activation buffer (20 μL). Variant proteases are added to a final concentration of 100 nM to 1 μM. The reaction is incubated at 37° C. for 1-2 hours and then separated by SDS-PAGE electrophoresis. Bands are visualized by Coomassie blue staining, silver staining, and/or Western blot.

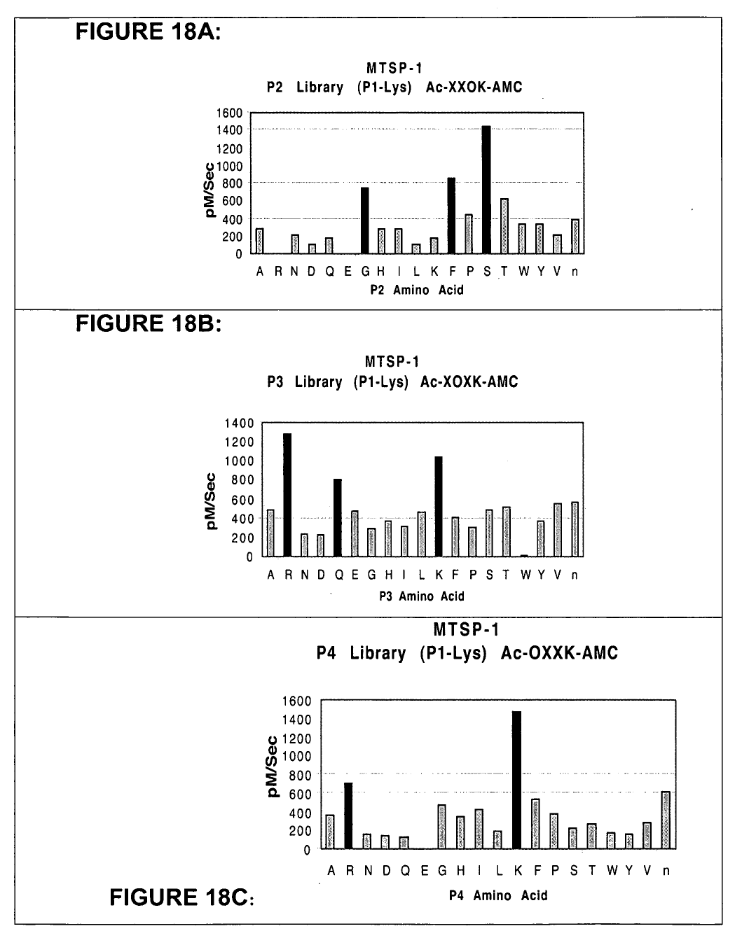

Result. The purified VEGFR2-Fc is efficiently cleaved by wild-type and mutant MT-SP1 (FIG. 18). Cleavage by variant proteases yields cleavage products with apparent molecular weights of ˜80 kDa and 30 kDa; analysis of potential cleavage sites in VEGFR2 suggests that MT-SP1 variants target the stalk (membrane proximal) region of VEGFR2. The mutant L172D cleaves full-length VEGFR2 but at a reduced rate compared to the wild-type. Several mutants (N175D and D217F) cleave the receptor with higher efficiency than wild-type. None of the protease variants or wild-type cleave the Fc domain.

### Example 24

### Assaying for Cleavage of VEGF Receptor from Endothelial Cells

Human umbilical vein endothelial cells (HUVECs) were purchased from Cambrex and cultured in EBM-2 (endothelial cell basal medium, Cambrex) with full supplements including 2% fetal calf serum (FCS) and antimycotics-antibiotics. For survival assays, cells were plated at a density of 2×10⁵ cells/ml in EBM-2 into 96-well plates overnight. The next day, cells were serum-starved by replacing the media with DMEM+10% FCS for 24 hours. Proteases were then added at varying concentrations from 10-1000 nM and the cells were incubated in the presence of the proteases for 2 hours. VEGF was added at a final concentration of 20 ng/mL and the cells were incubated for 72 hours. At the end of the 72 hours, cell count was determined by MTT assay (Sigma) according to the manufacturer's protocol.

To visualize the cleavage of the VEGF receptor from the surface of endothelial cells, cells were grown to ˜70% confluence in 24-well plates, at which point the media was removed and 200 uL of DMEM plus 10% FCS was added to each well. Proteases to be tested were added at final concentrations of 100-1000 nM. Cells were incubated in the presence of the proteases for 1-3 hours and the media was removed. Cells were washed with 1 mL ice cold PBS (3 times) and were scraped off the plate using a pipette tip. The resuspended cells were centrifuged at 5000 rpm and the supernatant was removed. The cells were lysed in 50 uL lysis buffer (PBS+0.1% NP40) by three freeze-thaw cycles on dry ice. The cell solution was centrifuged at 15,000 rpm to remove membranes and organelles, and 30 uL of the supernatant was separated by SDS gel electrophoresis. Proteins were transferred to a PVDF membrane and probed with an anti-VEGFR2 antibody recognizing the intracellular domain (Chemicon).

Release of the soluble VEGF receptor from the surface of endothelial cells by proteolytic cleavage was detected using a sandwich ELISA. HUVECs were grown in 24-well plates and treated with proteases as described above. After 3 hours incubation, 100 uL of media was removed and the protease inhibitor Pefabloc (Roche) was added to a final concentration of 1 mg/mL. The media was then added to Maxisorp plates (Nunc) that had been treated with a monoclonal antibody recognizing the extracellular domain of VEGFR2 (MAB3573, R & D Systems, 1:125 dilution in PBS). After 1 hour incubation, the plates were washed with PBS+0.01% Tween 20 (PBST), and were treated with a biotinylated polyclonal antibody also recognizing the extracellular domain (BAF357, R & D Systems, 1:500 dilution). After 1 hour incubation plates were washed with PBST and then treated with strepavidin conjugated horseradish peroxidase (Upstate). Plates were incubated for 1 hour and then washed with PBST, and developed using TMB substrate (Amersham) according to the manufacturer's protocol.

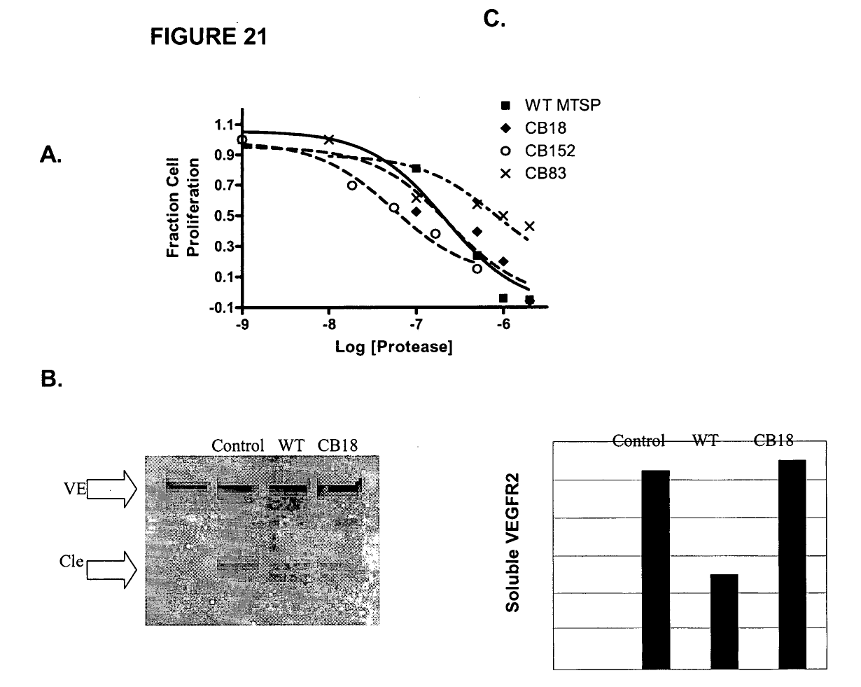

Results. Wild-type MT-SP1 and the more specific mutants, including CB18, CB83 and CB152, efficiently inhibited VEGF-dependent proliferation of endothelial cells in a dose-dependent manner (FIG. 21A). Consistent with the prediction that the MT-SP1 variants inhibit VEGF-dependent cell proliferation by inactivating the VEGF receptor, FIG. 21B shows that the MT-SP1 variants cleave the VEGF receptor on the surface of endothelial cells. Shown is a Western blot in which HUVECs are incubated with the buffer control or MT-SP1 variants, and then cell extract are probed with an antibody recognizing the intracellular domain of VEGFR2. Wild-type MT-SP1 and variants cleave the full-length receptor (upper band) to generate a truncated form (lower band). In addition, the extracellular domain (ectodomain) of the cleaved receptor can be detected in the media, as shown by the ELISA in FIG. 21C; the released ectodomain is detectable in samples treated with MT-SP1 and variants, but not in the control.

### Example 25

### Cornea Micropocket Model

To determine the acute maximum tolerated dose, escalating doses of purified wild-type and variant MT-SP1s were injected i.v into C57BL/6 mice. The mice were observed for outward signs of toxicity, and death.

For the cornea micropocket assay, C57BL/6 mice are anesthetized with avertin i.p. and the eye was treated with topical proparacaine HCl (Allergan, Irvine, Calif.). Hydron/sucralfate pellets containing VEGF-A₁₆₅ (100 ug, R & D Systems) were implanted into a corneal micropocket at 1 mm from the limbus of both eyes under an operating microscope (Zeiss) followed by intrastomal linear keratotomy by using a microknife (Medtroni Xomed, Jacksonville, Fla.). A corneal micropocket was dissected toward the limbus with a von Graefe knife #3 (2×30 mm), followed by pellet implantation and application of topical erythromycin. After 8 days, neovascularization is quantitated by using a slit lamp biomicroscope and the formula 2π×(vessel length/10)×(clock hours). P values were determined by using a two-tailed t test assuming unequal variances (Microsoft EXCEL). Varying doses of proteases were injected by i.p. twice a day at 12 hour intervals starting at day 0 until day 7.

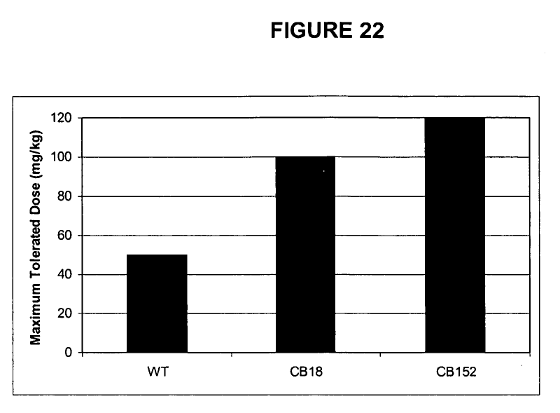

Results. Wild-type MT-SP1 was well tolerated by mice, with an acute maximum tolerated dose (MTD) determined to be 50 mg/kg (FIG. 22). Significantly, some of the MT-SP1 variants that were shown to have narrower selectivity in the profiling libraries (see FIG. 16) were better tolerated (i.e. had lower toxicities), resulting in higher maximum tolerated doses. CB18 and CB152, for instance, were tolerated at doses that resulted in death for wild-type MT-SP1. This demonstrates that narrowing the selectivity can be a mechanism for reducing the toxicity of protease drugs.

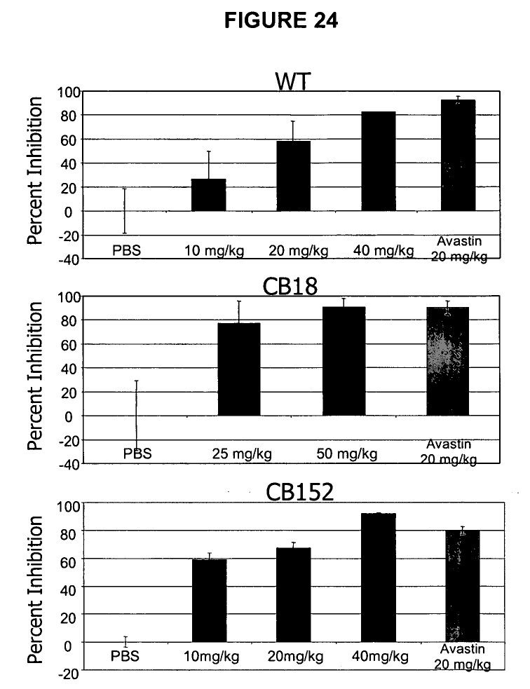

Wild-type MT-SP1 and variants were tested for their ability to inhibit VEGF-induced angiogenesis in the mouse cornea micropocket model. As outlined above, a pellet of VEGF was implanted into the cornea of mice, which is normally avascular, and the amount of neovascularization was quantitated after 8 days. When mice were treated with either wild-type or variant MT-SP1, neovascularization was inhibited in a dose dependent manner (FIG. 24). Treatment of mice with wild-type MT-SP1 at the MTD (50 mg/kg) resulted in 42% inhibition of neovascularization. In the case of CB18, it was possible to dose at a higher concentration due to the lower toxicity, and at the higher dose (80 mg/kg) an inhibition of 75% was achieved. Thus, even though wild-type MT-SP1 was effective at inhibiting VEGF-induced angiogenesis, better efficacy was obtained with CB18 due to the fact that it could be dosed at a higher level.

### Example 26

### Miles Assay for Vascular Permeability

In addition to angiogenesis, VEGF also induces the permeability of blood vessels, resulting in the leakage of fluids into the surrounding tissue. VEGF-induced vascular permeability was measured using the Miles assay. Briefly, nude (athymic) mice were injected with 0.5% Evan's blue dye (100 uL in PBS, Sigma) by tail vein injection. One hour after dye injection, 100 ng of VEGF in 20 uL PBS was injected intradermally into the back of the mice in duplicate spots. Vascular permeability is visualized by the appearance of blue spots at the site of VEGF injection due to the leakage of the dye. The extent of vascular permeability can be measured semi-quantitatively by measuring the area of the blue spots. To determine if they inhibited vascular permeability, wild-type MT-SP1 and variants were injected i.p. at varying doses immediately after injection of the dye, and the amount of vascular permeability was determined by measuring the area of dye leakage.

Results. Injection of wild-type and variant MT-SP1 resulted in dose-dependent inhibition of vascular permeability (FIG. 24). At the highest dose tested, wild-type MT-SP1 inhibited vascular permeability up to 80%. Similarly, both CB18 and CB152 inhibited vascular permeability, with CB152 showing higher efficacy at the low 10 mg/kg dose than wild-type (60% inhibition for CB152 compared to 25% inhibition for wild-type). At their highest doses, all three proteases had comparable efficacy to AVASTIN™, an anti-VEGF antibody approved for colon cancer.

### Example 27

### Tumor Xenograft Model

Murine Lewis lung carcinoma (LLC) cells are passaged on the dorsal midline of C57BL/6 mice or in DMEM/10% FCS/penicillin/streptomycin (PNS)/L-glutamine. T241 murine fibrosarcoma is grown in DMEM/10% FCS/PNS/L-glutamine and human pancreatic BxPc3 adenocarcinoma in RPMI medium 1640/10% FCS/PNS. Tumor cells (10⁶) are injected s.c. into the dorsal midline of C57BL/6 mice (8-10 weeks old) for murine tumors and severe combined immunodeficient (SCID) mice for human tumors, grown to 100-200 mm³ (typically 10-14 days) to demonstrate tumor take, and 10⁹ pfu of protease-encoding adenoviruses or the control adenovirus Ad Fc given by i.v. tail-vein injection. Tumor size mm³ is calculated by caliper measurements over a 10- to 14-day period by using the formula 0.52×length (mm)×width (mm), using width as the smaller dimension. See, e.g., Kuo et al., PNAS, 2001, 98:4605-4610. P values were determined by using a two-tailed t test assuming unequal variances (Microsoft EXCEL).

Results. Given that cleavage of VEGFR2 will inactivate the receptor, then the systemic delivery of therapeutically effective amounts of protease—either as purified protein or encoded by adenovirus—will result in inhibition of LLC tumor growth. Failure to inhibit tumor growth may be due to the inactivation of the protease by endogenous protease inhibitors (serpins). In such an event, the covalent binding of the serpin to the protease will be detectable as an increase in size of the protease by SDS-PAGE. Mutations can be made in the protease that will make it resistant to serpin inactivation.

### Example 28

### VEGFR Cleavage

As shown in FIG. 15, scaffold proteases and variants have been successfully expressed as active proteases in yeast or bacterial expression systems at multi-milligram quantities. See, e.g., protocols described in Harris 1998 and Takeuchi, 2000. MT-SP1 was engineered to obtain muteins that selectively cleave Flk-1/KDR.

Additional MT-SP1 muteins, shown in Table 17, where cloned and expressed as described above. As shown in FIG. 15, MT-SP1 variants were expressed in bacteria and purified from inclusion bodies. Each protease retains high catalytic activity and is >99% pure making them appropriate for crystallographic studies.

Table 25 depicts the potential target cleavage sequences for wild-type and mutein MT-SP1. In the table, “Hyd” represents any hydrophobic amino acid (i.e. glycine, alanine, valine, leucine, isoleucine, phenylalanine, tyrosine, or tryptophan) and “Xxx” represents any amino acid. “Xxx” represents any amino acid.

TABLE 25
Potential MT-SP1 Cleavage Sequences

| | P4 | P3 | P2 | P1 | SEQ ID NO |
|---|---|---|---|---|---|
| MT-SP1 | | | | | |
| Native specificity | K/R | Hyd | Xxx | K/R | 10 |
| | Hyd | K/R | Xxx | K/R | 11 |
| VEGFR2 sequences | K | V | G | R | 12 |
| | R | V | R | K | 13 |
| | R | R | V | R | 14 |
| | R | K | T | K | 15 |
| | K | T | K | K | 16 |
| | T | K | K | R | 17 |

### Example 29

### Muteins Consisting of One, Two and Three Mutations with Increased Selectivity Towards VEGFR Stalk Region Sequence, RRVR

Multiple muteins were characterized by PSSCL profiling showing increased selectivity towards the RRVR target cleavage sequence (FIG. 16A-H). They were grouped into two sub-classes based on which subsite profile was most affected by the mutation: P2 or P3&P4. Mutations of Phe99 to Ala, Ile, and Val increased the protease's P2 selectivity towards Val, and reduced the specificity of Ala containing substrates. This effect is seen in the variants F99V MT-SP1 (CB38), and F99I/L172D/Q175D MT-SP1 (CB159) (FIG. 16C&D). Mutations such as Phe99 to Trp, Asn, Asp, Ala, or Arg increased the P2 selectivity for Ala, Ser, Trp, Lys and Ile containing substrates. Additional mutations that affected the P2 selectivity were Met180 to Glu and Ala and Trp215 to Tyr and Phe.

Mutation of Gln192 to Arg and Glu altered the P3 selectivity alone. Mutations at Tyr 146 (Asp), Leu172 (Asp), Gln175 (Asp), Lys224 (Phe), and Met180 (Glu) increased the selectivity of the variants, towards both P3 and P4 Arg and Lys containing substrates as in variant L172D (CB18) (FIG. 16B). Grouping these individual mutations together resulted in variant proteases with highly selective P3 and P4 profiles such as the variants L172D/Q175D (CB83) and Y146D/K224F (CB155) (FIG. 162E&F).

Results. By grouping mutations identified individually to narrow the protease selectivity at P2 and at P3/P4, multiple variants were made that had greater than four fold selectivity towards Arg and Lys residues at the P3 and P4 positions, and altered P2 specificity. Two variants F99V/L172D/Q175D (CB151) and F99V/K224F (CB152) are at least 3 fold more selective of Arg and Lys than other amino acids at the P3 and P4 subsites, and twice as selective for Val over Ala at the P2 subsite (FIG. 16G&H). These characteristics in the PSSCL demonstrate the efficacy of mutations from Table 17 on altering the selectivity of the MT-SP1 protease towards the desired RRVR sequence.

### Example 30

### Screening for Preferential Cleavage of RRVR Versus RQAR Substrates

Mutant proteases that match the desired specificity profiles, as determined by substrate libraries, were assayed using individual peptide substrates corresponding to the desired cleavage sequence to determine the magnitude of the change in selectivity. Two substrates were designed: Ac-RRVR-AMC and Ac-RQAR-AMC. The second sequence, RQAR, is a preferred sequence of MT-SP1 as determined by substrate profiling. It also matches the sequence in the full length protease that must be cleaved for protease activation.

Michealis-Menton kinetic constants were determined by the standard kinetic methods. Briefly, the substrate is diluted in a series of 12 concentrations between 1 mM and 2 μM in 50 μL total volume of MT-SP1 activity buffer in the wells of a Costar 96 well black half-area assay plate. The solution is warmed to 30° C. for five minutes, and 50 μL of a protease solution between 0.1 and 20 nM was added to the wells of the assay. The fluorescence was measured in a fluorescence spectrophotometer (Molecular Devices Gemini XPS) at an excitation wavelength of 380 nm, an emission wavelength of 450 nm and using a cut-off filter ser at 435 nm. The rate of increase in fluorescence was measured over 30 minutes with readings taken at 30 second intervals. The kinetic constants k_{cat}, Kₘ and k_{cat}/Kₘ were calculated by graphing the inverse of the substrate concentration versus the inverse of the velocity of substrate cleavage, and fitting to the Lineweaver-Burk equation (1/velocity=(Kₘ/Vₘₐₓ)(1/[S])+1/Vₘₐₓ; where Vₘₐₓ=[E]*k_{cat}). The specificity constant (k_{cat}/Kₘ) is a measure of how well a substrate is cut by a particular protease.

Results: The specificity constants (k_{cat}/Kₘ) for wild-type MT-SP1 and seven variants (FIG. 21) demonstrate that the semi-quantitative results for relative selectivity between RQAR and RRVR derived from the PSSCL are consistent when measured for individual substrates. The wild-type protease, MT-SP1, prefers the RQAR substrate two times more than the RRVR substrate. Five of the six variant proteases prefer the target sequence RRVR over RQAR. Two variants, CB152 and CB159, prefer RRVR to RQAR by greater than 8 fold. The only exception is CB38 where the profile suggested that the selectivity was exclusively at the P4 subsite. In addition to the relative preference of RQAR versus RRVR, individual substrate kinetic measurements define the efficiency of substrate cleavage for each variant. The variants CB155 and CB159 cut the Ac-RRVR-AMC substrate at 2.2 and 2.3×10⁵ M⁻¹s⁻¹, respectively (FIG. 21). These rates are within 3 fold of the wild-type, MT-SP1.

### Example 31

### Screening for Cleavage of Individual Substrates

Mutant proteases that match the desired specificity profiles, as determined for example, by substrate libraries, are assayed using individual peptide substrates corresponding to the desired cleavage sequence. Individual kinetic measurements are performed using a Spectra-Max Delta fluorimeter (Molecular Devices). Each protease is diluted to between 50 nM and 1 μM in assay buffer. All ACC substrates are diluted with MeSO to between 5 and 500 μM, while AMC substrates are diluted to between 20 and 2000 μM. Each assay contain less than 5% MeSO. Enzymatic activity is monitored every 15 seconds at excitation and emission wavelengths of 380 nm and 460 nm, respectively, for a total of 10 minutes. All assays are performed in 1% DMSO.

### Example 32

### Screening for Cleavage of Full-Length Proteins

Variant proteases are assayed to ascertain that they will cleave the desired sequence when presented in the context of the full-length protein, and the activity of the target protein is assayed to verify that its function has been destroyed by the cleavage event. The cleavage event is monitored by SDS-PAGE after incubating the purified full-length protein with the variant protease. The protein is visualized using standard Coomasie blue staining, by autoradiography using radio labeled protein, or by Western blot using the appropriate antibody. Alternatively, if the target protein is a cell surface receptor, cells expressing the target protein are exposed to the variant protease. The cleavage event is monitored by lysing the cells and then separating the proteins by SDS-PAGE, followed by visualization by Western blot. Alternatively, the soluble receptor released by proteolysis is quantified by ELISA.

Cleavage of VEGF.

Vascular endothelial growth factor (VEGF) is an endothelial cell-specific mitogen normally produced during embryogenesis and adult life. VEGF is a significant mediator of angiogenesis in a variety of normal and pathological processes, including tumor development. Three high affinity cognate receptors to VEGF have been identified: VEGFR-1/Flt-1, VEGFR-2/KDR, and VEGFR-3/Flt-4.

To determine if MT-SP1 cleaves both the signaling molecule in addition to the receptor, a 165 amino acid recombinant version of VEGF, VEGF⁶⁵, was assayed by SDS-PAGE. VEGF¹⁶⁵ was reconstituted in PBS to a concentration of 0.2 μg/μL and diluted to a final concentration of 5 μM. Solutions with no protease and 100 nM MT-SP1 or CB152 were incubated with the VEGF at 37° C. for five hours. The resulting protein cleavage products were deglycosylated, separated by SDS-PAGE, and silver stained (FIG. 26). MT-SP1 efficiently cleaves VEGF¹⁶⁵ under the assay conditions while the more selective variant CB152 does not. This result demonstrates that wild-type MT-SP1 can be used to block VEGF signaling through two different mechanisms: cleavage of the mitogen and cleavage of the receptor. CB152, a variant with narrow selectivity to the RRVR sequence in the stalk region of VEGFR2, does not cleave VEGF, but does cleave VEGFR and can be dosed at higher concentrations due to reduced toxicity.

Cleavage Of VEGFR.

¹²⁵I-VEGFR (40,000 cpm) is incubated with varying concentrations of protease, samples are boiled in SDS-PAGE sample buffer and examined on a 12% polyacrylamide gel. The gels are dried and exposed to x-ray film(Kodak) at −70° C.

VEGFR Binding Assay.

¹²⁵I-VEGFR or PMN are incubated with varying concentrations of proteases as above. The binding of ¹²⁵I-VEGFR exposed to proteases to normal PMN, or the binding of normal ¹²⁵I-VEGFR to PMN exposed to proteases, are quantified using scintillation. Briefly, 10⁵ cells are incubated with varying concentrations of ¹²⁵I-VEGFR in 96-well filter plates (Millipore) in the presence of protease inhibitors. Cells are washed three times by vacuum aspiration and 30 μL of scintillation fluid (Wallac) are added to each well. Scintillation are counted on a Wallac Microbeta scintillation counter, (adapted from van Kessel et al., J. Immunol. (1991) 147: 3862-3868 and Porteau et al., JBC (1991) 266:18846-18853).

### Example 33

### Measuring Activity of MT-SP1 in Serum

The activity of MT-SP1 and trypsin was assayed in the presence of increasing concentrations of fetal calf serum. The high concentrations of macromolecular protease inhibitors present in serum makes it a good in vitro system to test whether a protease would be active in vivo. MT-SP1 and trypsin were resuspended in Dulbecco's Modified Eagle's Medium (DMEM) at 100 nM and 80 nM, respectively, with increasing serum concentrations (0-10%) in a final volume of 100 μL. A fluorogenic peptide substrate (Leu-Val-Arg-aminomethylcoumarin) was added to a final concentration of 15 μM and fluorescence was detected in a fluorescence plate reader (Molecular Devices) with an excitation wavelength of 380 nm and an emission wavelength of 460 nm.

As shown in FIG. 19, trypsin shows very strong activity in 0% serum, with the enzyme using up all the substrate after ˜400 seconds. However, even in the lowest concentration of serum (2.5%), trypsin activity is drastically reduced, presumably due to the binding of macromolecular protease inhibitors. MT-SP1, on the other hand, shows virtually the same activity in all concentrations of serum, suggestive that there are no endogenous protease inhibitors in serum that inactivate MT-SP1.

### Equivalents

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of screening method, protease scaffold, or library type is believed to be a matter of routine for a person of ordinary skill in the art with knowledge of the embodiments described herein. Other aspects, advantages, and modifications considered to be within the scope of the following claims.

What is claimed is:

1. A composition comprising a serine protease scaffold, wherein the protease comprises N mutated residues relative to a wild-type scaffold sequence, wherein the mutated residue modifies the target specificity of the protease, wherein N comprises a positive integer and wherein the protease cleaves VEGF or VEGF receptor.

2. The composition of claim 1, wherein N is any number between 1 and 20.

3. The composition of claim 1, wherein the serine protease scaffold comprises a polypeptide 95% identical to the amino acid sequence of wild-type granzyme B of SEQ ID NO:24, wherein the polypeptide has at least one mutation at one or more of the positions chosen from the group consisting of, 57, 58, 59, 60, 61, 62, 63, 97, 98, 99, 100, 102, 151, 169, 170, 171, 171A, 172, 173, 174, 175, 176, 177, 178, 179, 180, 181, 189, 190, 191, 192, 195, 215, 217 and 218, wherein the numbering is for chymotrypsin.

4. The composition of claim 3, wherein said mutation comprises a member of the group consisting of CB01, CB02, CB05, CB06 and CB10.

5. The composition of claim 1, wherein the serine protease scaffold comprises a polypeptide 95% identical to the amino acid sequence of wild-type MT-SP1 of SEQ ID NO:1, wherein the polypeptide has at least one mutation at one or more of the positions 171, 174, 180, 215, 192, 218, 99, 57, 189, 190, 226, 146, 172, 175, 41, 58, 59, 60, 61, 62, 63, 97, 98, 100, 102, 151, 169, 170, 171A, 173, 176, 177, 178, 179, 181, 191, 195 or 224 or 217, wherein the numbering is for chymotrypsin.

6. The composition of claim 1, wherein said mutation is a member of the group consisting of L172D, Y146F, N175D, D217F, F99V and K224F.

7. The composition of claim 5, wherein said mutations are F99V and K224F.

8. The composition of claim 1, wherein cleavage of the VEGFR inhibits tumor-specific angiogenesis

9. A composition comprising a mutein granzyme B, wherein the mutein granzyme B cleaves a substrate recognition site of P4-P3-P2-P1, wherein the P4-P3-P2-P1 site comprises a four amino acid sequence found on a VEGF or a VEGFR.

10. The composition of claim 9, wherein P4-P3-P2-P1 is a recognition site located in the extracellular domain of a VEGFR.

11. The composition of claim 9, wherein the substrate recognition site of P4-P3-P2-P1 comprises an amino acid sequence selected from the group consisting of VLKD, LVED, WFKD and RIYD.

12. The composition of claim 10, wherein the VEGFR is VEGF-R2/flk-1/KDR.

13. A composition comprising a mutein granzyme B, wherein the mutein Granzyme B cleaves a target polypeptide comprising the protease recognition site that is not recognized by a wild-type granzyme B.

14. The composition of claim 13, wherein the mutein granzyme B protease target specificity is increased by at least 2-fold compared to the wild-type granzyme B protease.

15. A composition comprising a mutein MT-SP1, wherein the mutein MT-SP1 cleaves a substrate recognition site of P4-P3-P2-P1, wherein the P4-P3-P2-P1 site comprises a four amino acid sequence found on a VEGFR.

16. The composition of claim 15, wherein the cleavage by the mutein is more selective for one of the protease recognition site of VEGFR as compared to the wild-type MT-SP1 protease recognition site.

17. The composition of claim 15, wherein the substrate recognition site of P4-P3-P2-P1 comprises an amino acid sequence selected from the group consisting of RRVR, KVGR, RVRK, RKTK, KTKK and KTTR.

18. The composition of claim 15, wherein the substrate recognition site of P4-P3-P2-P1 comprises an amino acid sequence of RRVR.

19. The composition of claim 15, wherein the VEGFR is VEGF-R2/flk-1/KDR.

20. The composition of claim 15, wherein the mutein granzyme B protease target specificity is increased by at least 2-fold compared to the wild-type granzyme B protease.

21. A method of treating cancer, said method comprising administering to a subject in need thereof a therapeutically efficient amount of a mutein serine protease that cleaves a vascular endothelial growth factor (VEGF) or a vascular endothelial growth factor receptor (VEGFR) substrate sequence involved in cancer, thereby treating the cancer.

22. The method of claim 21, wherein the patient is a mammal.

23. The method of claim 21, wherein the patient is a human.

24. The method of claim 21, wherein the mutein is based on a MT-SP1 or granzyme B protease scaffold.

25. The composition of claim 21, wherein P4-P3-P2-P1 is a recognition site located in the extracellular domain of a VEGFR.

26. The composition of claim 21, wherein the VEGFR is VEGF-R2/flk-1 /KDR.

27. The method of claim 21, wherein the protease is administered in combination with an anti-cancer agent.

28. The method of claim 21, wherein the protease is administered in combination with an anti-cancer treatment.

29. The method of claim 21, wherein cleavage of the VEGF or VEGFR inhibits tumor-specific angiogenesis.


SEQUENCE LISTING (43 sequences)

SEQ ID NO: 1 - protein, 227 aa, Homo sapiens
IIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 2 - protein, 226 aa, Homo sapiens
IIGGHEAKPHSRPYMAFVQFLQEKSRKRCGGILVRKDFVLTAAHCQGSSINVTLGAHNIKEQERTQQFIPVKRPIPHPAYNPKNFSNDIMLLQLERKAKWTTAVRPLRLPSSKAQVKPGQLCSVAGWGYVSMSTLATTLQEVLLTVQKDCQCERLFHGNYSRATEICVGDPKKTQTGFKGDSGGPLVCKDVAQGILSYGNKKGTPPGVYIKVSHFLPWIKRTMKRL

SEQ ID NO: 3 - protein, 228 aa, Rattus norvegicus
IIGGHEADPHSRPYMAYLQYKNEDSRDTICGGFLIREDFVLTAAHCSGSKINVTLGAHNIKEQEKTQQVIPVVKIIPHPAYNAKTISNDIMLLKLKSKAKRTRAVKTLSLPRSNFKVKPGDVCYVAGWGKLGPMGKFPDKLQEVELTVQEDQECETYLKNAYDKANQICAGDPKIKCASFQGDSGGPLVCKKVAAGIVSYGRKDGSTPRAFTKVSTFLSWIEETMKKS

SEQ ID NO: 4 - protein, 228 aa, Rattus norvegicus
IIGGHEAKPHSRPYMAYLQIMDEYSGSKKCGGFLIREDFVLTAAHCSGSKINVTLGAHNIKEQEKMQQIIPVVKIIPHPAYNSKTISNDIMLLKLKSKAKRSSAVKPLNLPRRNVKVKPGDVCYVAGWGKLGPMGKYSDTLQEVELTVQEDQKCESYLKNYFDKANEICAGDPKIKRASFRGDSGGPLVCKKVAAGIVSYGQNDGSTPRAFTKVSTFLSWIKKTMKKS

SEQ ID NO: 5 - protein, 227 aa, Mus musculus
IIGGHEVKPHSRPYMALLSIKDQQPEAICGGFLIREDFVLTAAHCEGSIINVTLGAHNIKEQEKTQQVIPMVKCIPHPDYNPKTFSNDIMLLKLKSKAKRTRAVRPLNLPRRNVNVKPGDVCYVAGWGRMAPMGKYSNTLQEVELTVQKDRECESYFKNRYNKTNQICAGDPKTKRASFRGDSGGPLVCKKVAAGIVSYGYKDGSPPRAFTKVSSFLSWIKKTMKSS

SEQ ID NO: 6 - protein, 228 aa, Mus musculus
IIGGNEISPHSRPYMAYYEFLKVGGKKMFCGGFLVRDKFVLTAAHCKGSSMTVTLGAHNIKAKEETQQIIPVAKAIPHPDYNPDDRSNDIMLLKLVRNAKRTRAVRPLNLPRRNAHVKPGDECYVAGWGKVTPDGEFPKTLHEVKLTVQKDQVCESQFQSSYNRANEICVGDSKIKGASFEEDSGGPLVCKRAAAGIVSYGQTDGSAPQVFTRVLSFVSWIKKTMKHS

SEQ ID NO: 7 - protein, 228 aa, Mus musculus
IIGGHVVKPHSRPYMAFVMSVDIKGNRIYCGGFLIQDDFVLTAAHCKNSSMTVTLGAHNITAKEETQQIIPVAKDIPHPDYNATIFYSDIMLLKLESKAKRTKAVRPLKLPRSNARVKPGDVCSVAGWGSRSINDTKASARLREVQLVIQEDEECKKRFRYYTETTEICAGDLKKIKTPFKGDSGGPLVCHNQAYGLFAYAKNGTISSGIFTKVVHFLPWISWNMKLL

SEQ ID NO: 8 - protein, 228 aa, Mus musculus
IIGGHVVKPHSRPYMAFVKSVDIEGNRRYCGGFLVQDDFVLTAAHCRNRTMTVTLGAHNIKAKEETQQIIPVAKAIPHPDYNATAFFSDIMLLKLESKAKRTKAVRPLKLPRPNARVKPGDVCSVAGWGSRSINDTKASARLREAQLVIQEDEECKKRFRHYTETTEICAGDLKKIKTPFKGDSGGPLVCDNKAYGLLAYAKNRTISSGVFTKIVHFLPWISRNMKLL

SEQ ID NO: 9 - protein, 228 aa, Mus musculus
IIGGHEVKPHSRPYMAFIKSVDIEGKKKYCGGFLVQDDFVLTAAHCRNRSMTVTLGAHNIKAKEETQQIIPVAKAIPHPAFNRKHGTNDIMLLKLESKAKRTKAVRPLKLPRPNARVKPGDVCSVAGWGKTSINATKASARLREAQLIIQEDEECKKLWYTYSKTTQICAGDPKKVQAPYEGESGGPLVCDNLAYGVVSYGINRTITPGVFTKVVHFLPWISTNMKLL

SEQ ID NO: 10 - protein, 228 aa, Mus musculus
IIGGHEVKPHSRPYMARVRFVKDNGKRHSCGGFLVQDYFVLTAAHCTGSSMRVILGAHNIRAKEETQQIIPVAKAIPHPAYDDKDNTSDIMLLKLESKAKRTKAVRPLKLPRPNARVKPGHVCSVAGWGRTSINATQRSSCLREAQLIIQKDKECKKYFYKYFKTMQICAGDPKKIQSTYSGDSGGPLVCNNKAYGVLTYGLNRTIGPGVFTKVVHYLPWISRNMKLL

SEQ ID NO: 11 - protein, 228 aa, Rattus norvegicus
IIWGTESKPHSRPYMAFIKFYDSNSEPHHCGGFLVAKDIVMTAAHCNGRNIKVTLGAHNIKKQENTQVISVVKAKPHENYDRDSHFNDIMLLKLERKAQLNGVVKTIALPRSQDWVKPGQVCTVAGWGRLANCTSSNTLQEVNLEVQKGQKCQDMSEDYNDSIQLCVGNPSEGKATGKGDSGGPFVCDGVAQGIVSYRLCTGTLPRVFTRISSFIPWIQKTMKVLQQS

SEQ ID NO: 12 - protein, 235 aa, Homo sapiens
IIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 13 - protein, 236 aa, Mus musculus
IIGGREARPHSYPYMAFLLIQSPEGLSACGGFLVREDFVLTAAHCLGSSINVTLGAHNIQMRERTQQLITVLRAIRHPDYNPQNIRNDIMLLQLRRRARRSGSVKPVALPQASKKLQPGDLCTVAGWGRVSQSRGTNVLQEVQLRVQMDQMCANRFQFYNSQTQICVGNPRERKSAFRGDSGGPLVCSNVAQGIVSYGSNNGNPPAVFTKIQSFMPWIKRTMRRFAPRYQRPANSL

SEQ ID NO: 14 - protein, 226 aa, Mus musculus
IIGGVEARPHSRPYMAHLKIITDRGSEDRCGGFLIAPQFVLTAAHCKGREITVTLGAHDVSKSESTQQRIKVEKQIIHKNYNVSFNLYDIMLLKLEEKAELTPTVDVIPLPGPSDFIDPGKMCWTAGWGKTGEKEPTSETLREVELRIMDKEACKMYKHYDYNFQVCVGSSTKLKTAYMGDSGGPLLCAGVAHGIVSYGDSHGKPPAVFTRISAYVPWIKTVINGK

SEQ ID NO: 15 - protein, 227 aa, Rattus norvegicus
IIGGVESIPHSRPYMAHLDIVTEKGLRVICGGFLISRQFVLTAAHCKGREITVILGAHDVRKRESTQQKIKVEKQIIHESYNSVPNLHDIMLLKLEKKVELTPAVNVVPLPSPSDFIHPGAMCWAAGWGKTGVRDPTSYTLREVELRIMDEKACVDYRYYEYKFQVCVGSPTTLRAAFMGDSGGPLLCAGVAHGIVSYGHPDAKPPAIFTRVSTYVPWINAVINTSS

SEQ ID NO: 16 - protein, 237 aa, Rattus norvegicus
IIGGVESRPHSRPYMAHLEITTERGYKATCGGFLVTRQFVMTAAHCKGRETTVTLGVHDVSKTESTQQKIKVEKQIVHPNYNFYSNLHDIMLLKLQKKAKVTPAVDVIPLPQPSDFLKPGKMCRAAGWGQTGVTKPTSNTLREVKQRIMDKEACKNYFHYNYNFQVCVGSPRKIRSAYKGDSGGPLVCAGVAHGIVSYGRGDAKPPAVFTRISPYVPWINKVIKGKDLTSLSLHESE

SEQ ID NO: 17 - protein, 224 aa, Mus musculus
IIGGVEAKPHSRPYMAYLKFTTKNGSKERCGGFLIAPQFVMTAAHCNGSEISVILGAHNINKNEPTQQIIKTEKTFVHPKFQYLSGFYDIMLLKLQKKAELNSDVDVISLPSSSDFIKPGKMCWTAGWGKTGKNNPLSVTLREVELRIMDQEACKDHSDYDYQLQVCAGSPTTSKSIGQGDSGGPLVCDSVAHGIASSYEAKAPAVFTRISYYLPWIYKVLKSK

SEQ ID NO: 18 - protein, 226 aa, Homo sapiens
IIGGTECKPHSRPYMAYLEIVTSNGPSKFCGGFLIRRNFVLTAAHCAGRSITVTLGAHNITEEEDTWQKLEVIKQFRHPKYNTSTLHHDIMLLKLKEKASLTLAVGTLPFPSQFNFVPPGRMCRVAGWGRTGVLKPGSDTLQEVKLRLMDPQACSHFRDFDHNLQLCVGNPRKTKSAFKGDSGGPLLCAGVAQGIVSYGRSDAKPPAVFTRISHYRPWINQILQAN

SEQ ID NO: 19 - DNA, 726 bp, Homo sapiens
gttgttgggggcacggatgcggatgagggcgagtggccctggcaggtaagcctgcatgctctgggccagggccacatctgcggtgcttccctcatctctcccaactggctggtctctgccgcacactgctacatcgatgacagaggattcaggtactcagaccccacgcagtggacggccttcctgggcttgcacgaccagagccagcgcagcgcccctggggtgcaggagcgcaggctcaagcgcatcatctcccaccccttcttcaatgacttcaccttcgactatgacatcgcgctgctggagctggagaaaccggcagagtacagctccatggtgcggcccatctgcctgccggacgcctcccatgtcttccctgccggcaaggccatctgggtcacgggctggggacacacccagtatggaggcactggcgcgctgatcctgcaaaagggtgagatccgcgtcatcaaccagaccacctgcgagaacctcctgccgcagcagatcacgccgcgcatgatgtgcgtgggcttcctcagcggcggcgtggactcctgccagggtgattccgggggacccctgtccagcgtggaggcggatgggcggatcttccaggccggtgtggtgagctggggagacggctgcgctcagaggaacaagccaggcgtgtacacaaggctccctctgtttcgggactggatcaaagagaacactggggtatag

SEQ ID NO: 20 - protein, 247 aa, Homo sapiens
MQPILLLLAFLLLPRADAGEIIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 21 - protein, 248 aa, Rattus norvegicus
MKLLLLLLSFSLAPKTEAGEIIGGHEAKPHSRPYMAYLQIMDEYSGSKKCGGFLIREDFVLTAAHCSGSKINVTLGAHNIKEQEKMQQIIPVVKIIPHPAYNSKTISNDIMLLKLKSKAKRSSAVKPLNLPRRNVKVKPGDVCYVAGWGKLGPMGKYSDTLQEVELTVQEDQKCESYLKNYFDKANEICAGDPKIKRASFRGDSGGPLVCKKVAAGIVSYGQNDGSTPRAFTKVSTFLSWIKKTMKKS

SEQ ID NO: 22 - protein, 855 aa, Homo sapiens [sequence omitted]

SEQ ID NO: 23 - protein, 258 aa, Homo sapiens
DEKDCDCGLRSFTRQARVVGGTDADEGEWPWQVSLHALGQGHICGASLISPNWLVSAAHCYIDDRGFRYSDPTQWTAFLGLHDQSQRSAPGVQERRLKRIISHPFFNDFTFDYDIALLELEKPAEYSSMVRPICLPDASHVFPAGKAIWVTGWGHTQYGGTGALILQKGEIRVINQTTCENLLPQQITPRMMCVGFLSGGVDSCQGDSGGPLSSVEADGRIFQAGVVSWGDGCAQRNKPGVYTRLPLFRDWIKENTGV

SEQ ID NO: 24 - protein, 230 aa, Homo sapiens
IVNGEDAVPGSWPWQVSLQDKTGFHFCGGSLISEDWVVTAAHCGVRTSDVVVAGEFDQGSDEENIQVLKIAKVFKNPKFSILTVNNDITLLKLATPARFSQTVSAVCLPSADDDFPAGTLCATTGWGKTKYNANKTPDKLQQAALPLLSNAECKKSWGRRITDVMICAGASGVSSCMGDSGGPLVCQKDGAWTLVGIVSWGSDTCSTSSPGVYARVTKLIPWVQKILAAN

SEQ ID NO: 25 - protein, 4 aa, Homo sapiens
IIGG

SEQ ID NO: 26 - protein, 4 aa, Homo sapiens
VVGG

SEQ ID NO: 27 - protein, 4 aa, Homo sapiens
IVGG

SEQ ID NO: 28 - protein, 764 aa, Homo sapiens
KQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 29 - protein, 592 aa, Homo sapiens
IIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 30 - DNA, 42 bp, Artificial Sequence
  I99A mutation Forward primer
ccagcgtataattctaagacagcctccaatgacatcatgctg

SEQ ID NO: 31 - DNA, 42 bp, Artificial Sequence
  I99A mutation Reverse primer
cagcatgatgtcattggaggctgtcttagaattatacgctgg

SEQ ID NO: 32 - protein, 6 aa, Artificial Sequence
  Substrate
XIEPDX

SEQ ID NO: 33 - protein, 6 aa, Artificial Sequence
  Substrate
XIKPDX

SEQ ID NO: 34 - protein, 6 aa, Artificial Sequence
  Substrate
XIETDX

SEQ ID NO: 35 - protein, 6 aa, Artificial Sequence
  Substrate
XIEFDX

SEQ ID NO: 36 - protein, 6 aa, Artificial Sequence
  Substrate
XLEFDX

SEQ ID NO: 37 - protein, 6 aa, Artificial Sequence
  Substrate
XLEPDX

SEQ ID NO: 38 - DNA, 42 bp, Artificial Sequence; identical to SEQ ID NO: 30
  I99A Plasmid sequence

SEQ ID NO: 39 - DNA, 42 bp, Artificial Sequence
  I99F Plasmid sequence
ccagcgtataattctaagacattctccaatgacatcatgctg

SEQ ID NO: 40 - DNA, 42 bp, Artificial Sequence
  I99R Plasmid sequence
ccagcgtataattctaagacaagatccaatgacatcatgctg

SEQ ID NO: 41 - DNA, 44 bp, Artificial Sequence
  Y174A Plasmid Sequence
gtgagtcctacttaaaaaatgctttcgacaaagccaatgagata

SEQ ID NO: 42 - protein, 6 aa, Artificial Sequence
  Substrate
XIAPDX

SEQ ID NO: 43 - DNA, 42 bp, Artificial Sequence
  N218A Plasmid sequence
atcgtctcctatggacaagctgatggttcaactccacgggca